(19) United States
(12) Patent Application Publication
(10) Pub. No.: US 2006/0194275 A1
(43) Pub. Date: Aug. 31, 2006

(54) Transporter and ion channels

(75) Inventors: Mariah R. Baughn, LOS ANGELES, CA (US); Vicki S. Elliott, San Jose, CA (US); April J A Hafalia, Daly City, CA (US); Junming Yang, San Jose, CA (US); Narinder K. Chawla, Union City, CA (US); Jayalaxmi Ramkumar, Fremont, CA (US); Ian J. Forsythe, Edmonton, CA (US); Yan Lu, Mountain View, CA (US); Y Tom Tang, San Jose, CA (US); Henry Yue, Sunnyvale, CA (US); Brigitte E. Raumann, Chicago, IL (US); Preeti G. Lal, Santa Clara, CA (US); Yalda Azimzai, Oakland, CA (US); Dyung Aina M. Lu, San Jose, CA (US); Ameena R. Gandhi, San Frnacisco, CA (US); Michael B. Thornton, Oakland, CA (US); Danniel B. Nguyen, San Jose, CA (US); Chandra S. Arvizu, San Diego, CA (US); Brooke M. Emerling, Chicago, IL (US); Anita Swarnakar, San Francisco, CA (US); Monique G. Yao, Mountain View, CA (US); Li Ding, Creve Coeur, MO (US); Ann He, San Jose, CA (US); Jennifer A. Griffin, Fremont, CA (US); Madhusudan M. Sanjanwala, Los Altos, CA (US); Kimberly J. Gietzen, San Jose, CA (US); Ernestine A. Lee, Kensington, CA (US); Yuming Xu, Mountain View, CA (US); Janice K. Au-Young, Brisbane, CA (US); Debopriya Das, Oyster Bay, NY (US); Soo Yeun Lee, Mountain View, CA (US); Hsin-Ru Chang, Belmont, CA (US)

Correspondence Address: INCYTE CORPORATION;EXPERIMENTAL STATION, ROUTE 141 & HENRY CLAY ROAD, BLDG. E336, WILMINGTON, DE, 19880, US

(73) Assignee: INCYTE CORPORATION, PALO ALTO, CA (US)

(21) Appl. No.: 10/474,894

(22) PCT Filed: Apr. 12, 2002
(86) PCT No.: PCT/US02/11760

§ 371 Date: Jun. 25, 2004

Related U.S. Application Data
(62) Division of application No. 60/283,709, filed on Apr. 13, 2001.

(51) Int. Cl. C07K 14/705; C07H 21/04; C12P 21/06

(52) U.S. Cl. 435/69.1; 435/320.1; 435/325; 530/350; 536/23.5

(57) ABSTRACT

The invention provides human transporters and ion channels (TRICH) and polynucleotides which identify and encode TRICH. The invention also provides expression vectors, host cells, antibodies, agonists, and antagonists. The invention also provides methods for diagnosing, treating, or preventing disorders associated with aberrant expression of TRICH.

### TECHNICAL FIELD

This invention relates to nucleic acid and amino acid sequences of transporters and ion channels and to the use of these sequences in the diagnosis, treatment, and prevention of transport, muscle, autoimmune/inflammatory, infectious, immune deficiencies, metabolism, reproductive, neurological, cardiovascular, eye, and cell proliferative disorders, including cancer, and in the assessment of the effects of exogenous compounds on the expression of nucleic acid and amino acid sequences of transporters and ion channels.

### BACKGROUND OF THE INVENTION

Eukaryotic cells are surrounded and subdivided into functionally distinct organelles by hydrophobic lipid bilayer membranes which are highly impermeable to most polar molecules. Cells and organelles require transport proteins to import and export essential nutrients and metal ions including K⁺, NH₄⁺, Pᵢ, SO₄²⁻, sugars, and vitamins, as well as various metabolic waste products. Transport proteins also play roles in antibiotic resistance, toxin secretion, ion balance, synaptic neurotransmission, kidney function, intestinal absorption, tumor growth, and other diverse cell functions (Griffith, J. and C. Sansom (1998) The Transporter Facts Book, Academic Press, San Diego Calif., pp. 3-29). Transport can occur by a passive concentration-dependent mechanism, or can be linked to an energy source such as ATP hydrolysis or an ion gradient. Proteins that function in transport include carrier proteins, which bind to a specific solute and undergo a conformational change that translocates the bound solute across the membrane, and channel proteins, which form hydrophilic pores that allow specific solutes to diffuse through the membrane down an electrochemical solute gradient.

Carrier proteins which transport a single solute from one side of the membrane to the other are called uniporters. In contrast, coupled transporters link the transfer of one solute with simultaneous or sequential transfer of a second solute, either in the same direction (symport) or in the opposite direction (antiport). For example, intestinal and kidney epithelium contains a variety of symporter systems driven by the sodium gradient that exists across the plasma membrane. Sodium moves into the cell down its electrochemical gradient and brings the solute into the cell with it. The sodium gradient that provides the driving force for solute uptake is maintained by the ubiquitous Na⁺/K⁺ ATPase system. Sodium-coupled transporters include the mammalian glucose transporter (SGLT1), iodide transporter (NIS), and multivitamin transporter (SMVT). All three transporters have twelve putative transmembrane segments, extracellular glycosylation sites, and cytoplasmically-oriented N— and C-termini. NIS plays a crucial role in the evaluation, diagnosis, and treatment of various thyroid pathologies because it is the molecular basis for radioiodide thyroid-imaging techniques and for specific targeting of radioisotopes to the thyroid gland (Levy, O. et al. (1997) Proc. Natl. Acad. Sci. USA 94:5568-5573). SMVT is expressed in the intestinal mucosa, kidney, and placenta, and is implicated in the transport of the water-soluble vitamins, e.g., biotin and pantothenate (Prasad, P. D. et al. (1998) J. Biol. Chem. 273:7501-7506).

One of the largest families of transporters is the major facilitator superfamily (MFS), also called the uniporter-symporter-antiporter family. MFS transporters are single polypeptide carriers that transport small solutes in response to ion gradients. Members of the MFS are found in all classes of living organisms, and include transporters for sugars, oligosaccharides, phosphates, nitrates, nucleosides, monocarboxylates, and drugs. MFS transporters found in eukaryotes all have a structure comprising 12 transmembrane segments (Pao, S. S. et al. (1998) Microbiol. Molec. Biol. Rev. 62:1-34). The largest family of MFS transporters is the sugar transporter family, which includes the seven glucose transporters (GLUT1-GLUT7) found in humans that are required for the transport of glucose and other hexose sugars. These glucose transport proteins have unique tissue distributions and physiological functions. GLUT1 provides many cell types with their basal glucose requirements and transports glucose across epithelial and endothelial barrier tissues; GLUT2 facilitates glucose uptake or efflux from the liver; GLUT3 regulates glucose supply to neurons; GLUT4 is responsible for insulin-regulated glucose disposal; and GLUT5 regulates fructose uptake into skeletal muscle. Defects in glucose transporters are involved in a recently identified neurological syndrome causing infantile seizures and developmental delay, as well as glycogen storage disease, Fanconi-Bickel syndrome, and non-insulin-dependent diabetes mellitus (Mueckler, M. (1994) Eur. J. Biochem. 219:713-725; Longo, N. and L. J. Elsas (1998) Adv. Pediatr. 45:293-313).

Monocarboxylate anion transporters are proton-coupled symporters with a broad substrate specificity that includes L-lactate, pyruvate, and the ketone bodies acetate, acetoacetate, and beta-hydroxybutyrate. At least seven isoforms have been identified to date. The isoforms are predicted to have twelve transmembrane (TM) helical domains with a large intracellular loop between TM6 and TM7, and play a critical role in maintaining intracellular pH by removing the protons that are produced stoichiometrically with lactate during glycolysis. The best characterized H⁺-monocarboxylate transporter is that of the erythrocyte membrane, which transports L-lactate and a wide range of other aliphatic monocarboxylates. Other cells possess H⁺-linked monocarboxylate transporters with differing substrate and inhibitor selectivities. In particular, cardiac muscle and tumor cells have transporters that differ in their Kₘ values for certain substrates, including stereoselectivity for L- over D-lactate, and in their sensitivity to inhibitors. There are Na⁺-monocarboxylate cotransporters on the luminal surface of intestinal and kidney epithelia, which allow the uptake of lactate, pyruvate, and ketone bodies in these tissues. In addition, there are specific and selective transporters for organic cations and organic anions in organs including the kidney, intestine and liver. Organic anion transporters are selective for hydrophobic, charged molecules with electron-attracting side groups. Organic cation transporters, such as the ammonium transporter, mediate the secretion of a variety of drugs and endogenous metabolites, and contribute to the maintenance of intercellular pH (Poole, R. C. and A. P. Halestrap (1993) Am. J. Physiol. 264:C761-C782; Price, N. T. et al. (1998) Biochem. J. 329:321-328; and Martinelle, K. and I. Haggstrom (1993) J. Biotechnol. 30:339-350).

ATP-binding cassette (ABC) transporters are members of a superfamily of membrane proteins that transport substances ranging from small molecules such as ions, sugars, amino acids, peptides, and phospholipids, to lipopeptides, large proteins, and complex hydrophobic drugs. ABC transporters consist of four modules: two nucleotide-binding domains (NBD), which hydrolyze ATP to supply the energy required for transport, and two membrane-spanning domains (MSD), each containing six putative transmembrane segments. These four modules may be encoded by a single gene, as is the case for the cystic fibrosis transmembrane regulator (CFTR), or by separate genes. When encoded by separate genes, each gene product contains a single NBD and MSD. These “half-molecules” form homo- and heterodimers, such as Tap1 and Tap2, the endoplasmic reticulum-based major histocompatibility (MHC) peptide transport system. Several genetic diseases are attributed to defects in ABC transporters, such as the following diseases and their corresponding proteins: cystic fibrosis (CFTR, an ion channel), adrenoleukodystrophy (adrenoleukodystrophy protein, ALDP), Zellweger syndrome (peroxisomal membrane protein-70, PMP70), and hyperinsulinemic hypoglycemia (sulfonylurea receptor, SUR). Overexpression of the multidrug resistance (MDR) protein, another ABC transporter, in human cancer cells makes the cells resistant to a variety of cytotoxic drugs used in chemotherapy (Taglicht, D. and S. Michaelis (1998) Meth. Enzymol. 292:130-162).

A number of metal ions such as iron, zinc, copper, cobalt, manganese, molybdenum, selenium, nickel, and chromium are important as cofactors for a number of enzymes. For example, copper is involved in hemoglobin synthesis, connective tissue metabolism, and bone development, by acting as a cofactor in oxidoreductases such as superoxide dismutase, ferroxidase (ceruloplasmin), and lysyl oxidase. Copper and other metal ions must be provided in the diet, and are absorbed by transporters in the gastrointestinal tract. Plasma proteins transport the metal ions to the liver and other target organs, where specific transporters move the ions into cells and cellular organelles as needed. Imbalances in metal ion metabolism have been associated with a number of disease states (Danks, D. M. (1986) J. Med. Genet. 23:99-106).

Transport of fatty acids across the plasma membrane can occur by diffusion, a high capacity, low affinity process. However, under normal physiological conditions a significant fraction of fatty acid transport appears to occur via a high affinity, low capacity protein-mediated transport process. Fatty acid transport protein (FATP), an integral membrane protein with four transmembrane segments, is expressed in tissues exhibiting high levels of plasma membrane fatty acid flux, such as muscle, heart, and adipose. Expression of FATP is upregulated in 3T3-L1 cells during adipose conversion, and expression in COS7 fibroblasts elevates uptake of long-chain fatty acids (Hui, T. Y. et al. (1998) J. Biol. Chem. 273:27420-27429).

Mitochondrial carrier proteins are transmembrane-spanning proteins which transport ions and charged metabolites between the cytosol and the mitochondrial matrix. Examples include the ADP, ATP carrier protein; the 2-oxoglutarate/malate carrier; the phosphate carrier protein; the pyruvate carrier; the dicarboxylate carrier which transports malate, succinate, fumarate, and phosphate; the tricarboxylate carrier which transports citrate and malate; and the Grave's disease carrier protein, a protein recognized by IgG in patients with active Grave's disease, an autoimmune disorder resulting in hyperthyroidism. Proteins in this family consist of three tandem repeats of an approximately 100 amino acid domain, each of which contains two transmembrane regions (Stryer, L. (1995) Biochemistry, W.H. Freeman and Company, New York N.Y., p. 551; PROSITE PDOC00189 Mitochondrial energy transfer proteins signature; Online Mendelian Inheritance in Man (OMIM) *275000 Graves Disease).

This class of transporters also includes the mitochondrial uncoupling proteins, which create proton leaks across the inner mitochondrial membrane, thus uncoupling oxidative phosphorylation from ATP synthesis. The result is energy dissipation in the form of heat. Mitochondrial uncoupling proteins have been implicated as modulators of thermoregulation and metabolic rate, and have been proposed as potential targets for drugs against metabolic diseases such as obesity (Ricquier, D. et al. (1999) J. Int. Med. 245:637-642).

Ion Channels

The electrical potential of a cell is generated and maintained by controlling the movement of ions across the plasma membrane. The movement of ions requires ion channels, which form ion-selective pores within the membrane. There are two basic types of ion channels, ion transporters and gated ion channels. Ion transporters utilize the energy obtained from ATP hydrolysis to actively transport an ion against the ion's concentration gradient. Gated ion channels allow passive flow of an ion down the ion's electrochemical gradient under restricted conditions. Together, these types of ion channels generate, maintain, and utilize an electrochemical gradient that is used in 1) electrical impulse conduction down the axon of a nerve cell, 2) transport of molecules into cells against concentration gradients, 3) initiation of muscle contraction, and 4) endocrine cell secretion.

Ion Transporters

Ion transporters generate and maintain the resting electrical potential of a cell. Utilizing the energy derived from ATP hydrolysis, they transport ions against the ion's concentration gradient. These transmembrane ATPases are divided into three families. The phosphorylated (P) class ion transporters, including Na⁺—K⁺ ATPase, Ca²⁺-ATPase, and H⁺-ATPase, are activated by a phosphorylation event. P-class ion transporters are responsible for maintaining resting potential distributions such that cytosolic concentrations of Na⁺ and Ca²⁺ are low and cytosolic concentration of K⁺ is high. The vacuolar (V) class of ion transporters includes H⁺ pumps on intracellular organelles, such as lysosomes and Golgi. V-class ion transporters are responsible for generating the low pH within the lumen of these organelles that is required for function. The coupling factor (F) class consists of H⁺ pumps in the mitochondria. F-class ion transporters utilize a proton gradient to generate ATP from ADP and inorganic phosphate (Pᵢ).

The P-ATPases are hexamers of a 100 kD subunit with ten transmembrane domains and several large cytoplasmic regions that may play a role in ion binding (Scarborough, G. A. (1999) Curr. Opin. Cell Biol. 11:517-522). The P-type ATPases include three subfamilies: one involved in transport of heavy metal ions such as Cu²⁺ or Cd²⁺ across a bilayer, another that transports non-heavy metal ions such as H⁺, Na⁺, K⁺, or Ca²⁺; and a third recently identified group responsible for transport of amphipathic molecules such as aminophospholipids. Most of these family members are highly expressed in the central nervous system, but have substantially different patterns of expression. One member of this family was identified as the gene mutated in two types of familial inherited cholestasis (Halleck, M. S. et al. (1999) Physiol. Genomics 1:139-150).

The V-ATPases are composed of two functional domains: the V₁ domain, a peripheral complex responsible for ATP hydrolysis; and the V₀ domain, an integral complex responsible for proton translocation across the membrane. The F-ATPases are structurally and evolutionarily related to the V-ATPases. The F-ATPase F₀ domain contains 12 copies of the c subunit, a highly hydrophobic protein composed of two transmembrane domains and containing a single buried carboxyl group in TM2 that is essential for proton transport. The V-ATPase V₀ domain contains three types of homologous c subunits with four or five transmembrane domains and the essential carboxyl group in TM4 or TM3. Both types of complex also contain a single a subunit that may be involved in regulating the pH dependence of activity (Forgac, M. (1999) J. Biol. Chem. 274:12951-12954).

The resting potential of the cell is utilized in many processes involving carrier proteins and gated ion channels. Carrier proteins utilize the resting potential to transport molecules into and out of the cell. Amino acid and glucose transport into many cells is linked to sodium ion co-transport (symport) so that the movement of Na⁺ down an electrochemical gradient drives transport of the other molecule up a concentration gradient. Similarly, cardiac muscle links transfer of Ca²⁺ out of the cell with transport of Na⁺ into the cell (antiport).

Gated Ion Channels

Gated ion channels control ion flow by regulating the opening and closing of pores. The ability to control ion flux through various gating mechanisms allows ion channels to mediate such diverse signaling and homeostatic functions as neuronal and endocrine signaling, muscle contraction, fertilization, and regulation of ion and pH balance. Gated ion channels are categorized according to the manner of regulating the gating function. Mechanically-gated channels open their pores in response to mechanical stress; voltage-gated channels (e.g., Na⁺, K⁺, Ca²⁺, and Cl⁻ channels) open their pores in response to changes in membrane potential; and ligand-gated channels (e.g., acetylcholine-, serotonin-, and glutamate-gated cation channels, and GABA- and glycine-gated chloride channels) open their pores in the presence of a specific ion, nucleotide, or neurotransmitter. The gating properties of a particular ion channel (i.e., its threshold for and duration of opening and closing) are sometimes modulated by association with auxiliary channel proteins and/or post translational modifications, such as phosphorylation.

Mechanically-gated or mechanosensitive ion channels act as transducers for the senses of touch, hearing, and balance, and also play important roles in cell volume regulation, smooth muscle contraction, and cardiac rhythm generation. A stretch-inactivated channel (SIC) was recently cloned from rat kidney. The SIC channel belongs to a group of channels which are activated by pressure or stress on the cell membrane and conduct both Ca²⁺ and Na⁺ (Suzuki, M. et al. (1999) J. Biol. Chem. 274:6330-6335).

The pore-forming subunits of the voltage-gated cation channels form a superfamily of ion channel proteins. The characteristic domain of these channel proteins comprises six transmembrane domains (S1-S6), a pore-forming region (P) located between S5 and S6, and intracellular amino and carboxy termini. In the Na⁺ and Ca²⁺ subfamilies, this domain is repeated four times, while in the K⁺ channel subfamily, each channel is formed from a tetramer of either identical or dissimilar subunits. The P region contains information specifying the ion selectivity for the channel. In the case of K⁺ channels, a GYG tripeptide is involved in this selectivity (Ishii, T. M. et al. (1997) Proc. Natl. Acad. Sci. USA 94:11651-11656).

Voltage-gated Na⁺ and K⁺ channels are necessary for the function of electrically excitable cells, such as nerve and muscle cells. Action potentials, which lead to neurotransmitter release and muscle contraction, arise from large, transient changes in the permeability of the membrane to Na⁺ and K⁺ ions. Depolarization of the membrane beyond the threshold level opens voltage-gated Na⁺ channels. Sodium ions flow into the cell, further depolarizing the membrane and opening more voltage-gated Na⁺ channels, which propagates the depolarization down the length of the cell. Depolarization also opens voltage-gated potassium channels. Consequently, potassium ions flow outward, which leads to repolarization of the membrane. Voltage-gated channels utilize charged residues in the fourth transmembrane segment (S4) to sense voltage change. The open state lasts only about 1 millisecond, at which time the channel spontaneously converts into an inactive state that cannot be opened irrespective of the membrane potential. Inactivation is mediated by the channel's N-terminus, which acts as a plug that closes the pore. The transition from an inactive to a closed state requires a return to resting potential.

Voltage-gated Na⁺ channels are heterotrimeric complexes composed of a 260 kDa pore-forming α subunit that associates with two smaller auxiliary subunits, β1 and β2. The β2 subunit is a integral membrane glycoprotein that contains an extracellular Ig domain, and its association with α and β1 subunits correlates with increased functional expression of the channel, a change in its gating properties, as well as an increase an whole cell capacitance due to an increase in membrane surface area (Isom, L. L. et al. (1995) Cell 83:433-442).

Non voltage-gated Na⁺ channels include the members of the amiloride-sensitive Na⁺ channel/degenerin (NaC/DEG) family. Channel subunits of this family are thought to consist of two transmembrane domains flanking a long extracellular loop, with the amino and carboxyl termini located within the cell. The NaC/DEG family includes the epithelial Na⁺ channel (ENaC) involved in Na⁺ reabsorption in epithelia including the airway, distal colon, cortical collecting duct of the kidney, and exocrine duct glands. Mutations in ENaC result in pseudohypoaldosteronism type 1 and Liddle's syndrome (pseudohyperaldosteronism). The NaC/DEG family also includes the recently characterized H⁺-gated cation channels or acid-sensing ion channels (ASIC). ASIC subunits are expressed in the brain and form heteromultimeric Na⁺-permeable channels. These channels require acid pH fluctuations for activation. ASIC subunits show homology to the degenerins, a family of mechanically-gated channels originally isolated from C. elegans. Mutations in the degenerins cause neurodegeneration. ASIC subunits may also have a role in neuronal function, or in pain perception, since tissue acidosis causes pain (Waldmann, R. and M. Lazdunski (1998) Curr. Opin. Neurobiol. 8:418-424; Eglen, R. M. et al. (1999) Trends Pharmacol. Sci. 20:337-342).

K⁺ channels are located in all cell types, and may be regulated by voltage, ATP concentration, or second messengers such as Ca²⁺ and cAMP. In non-excitable tissue, K⁺ channels are involved in protein synthesis, control of endocrine secretions, and the maintenance of osmotic equilibrium across membranes. In neurons and other excitable cells, in addition to regulating action potentials and repolarizing membranes, K⁺ channels are responsible for setting resting membrane potential. The cytosol contains non-diffusible anions and, to balance this net negative charge, the cell contains a Na⁺—K⁺ pump and ion channels that provide the redistribution of Na⁺, K⁺, and Cl⁻. The pump actively transports Na⁺ out of the cell and K⁺ into the cell in a 3:2 ratio. Ion channels in the plasma membrane allow K⁺ and Cl⁻ to flow by passive diffusion. Because of the high negative charge within the cytosol, Cl⁻ flows out of the cell. The flow of K⁺ is balanced by an electromotive force pulling K⁺ into the cell, and a K⁺ concentration gradient pushing K⁺ out of the cell. Thus, the resting membrane potential is primarily regulated by K⁺ flow (Salkoff, L. and T. Jegla (1995) Neuron 15:489-492).

Potassium channel subunits of the Shaker-like superfamily all have the characteristic six transmembrane/1 pore domain structure. Four subunits combine as homo- or heterotetramers to form functional K channels. These pore-forming subunits also associate with various cytoplasmic b subunits that alter channel inactivation kinetics. The Shaker-like channel family includes the voltage-gated K⁺ channels as well as the delayed rectifier type channels such as the human ether-a-go-go related gene (HERG) associated with long QT, a cardiac dysrythmia syndrome (Curran, M. E. (1998) Curr. Opin. Biotechnol. 9:565-572; Kaczorowski, G. J. and M. L. Garcia (1999) Curr. Opin. Chem. Biol. 3:448-458).

A second superfamily of K⁺ channels is composed of the inward rectifying channels (Kir). Kir channels have the property of preferentially conducting K⁺ currents in the inward direction. These proteins consist of a single potassium selective pore domain and two transmembrane domains, which correspond to the fifth and sixth transmembrane domains of voltage-gated K⁺ channels. Kir subunits also associate as tetramers. The Kir family includes ROMK1, mutations in which lead to Bartter syndrome, a renal tubular disorder. Kir channels are also involved in regulation of cardiac pacemaker activity, seizures and epilepsy, and insulin regulation (Doupnik, C. A. et al. (1995) Curr. Opin. Neurobiol. 5:268-277; Curran, supra).

The recently recognized TWIK K⁺ channel family includes the mammalian TWIK-1, TREK-1 and TASK proteins. Members of this family possess an overall structure with four transmembrane domains and two P domains. These proteins are probably involved in controlling the resting potential in a large set of cell types (Duprat, F. et al. (1997) EMBO J 16:5464-5471).

The voltage-gated Ca²⁺ channels have been classified into several subtypes based upon their electrophysiological and pharmacological characteristics. L-type Ca²⁺ channels are predominantly expressed in heart and skeletal muscle where they play an essential role in excitation-contraction coupling. T-type channels are important for cardiac pacemaker activity, while N-type and P/Q-type channels are involved in the control of neurotransmitter release in the central and peripheral nervous system. The L-type and N-type voltage-gated Ca²⁺ channels have been purified and, though their functions differ dramatically, they have similar subunit compositions. The channels are composed of three subunits. The α₁ subunit forms the membrane pore and voltage sensor, while the α₂δ and β subunits modulate the voltage-dependence, gating properties, and the current amplitude of the channel. These subunits are encoded by at least six α₁, one α₂δ, and four β genes. A fourth subunit, γ, has been identified in skeletal muscle (Walker, D. et al. (1998) J. Biol. Chem. 273:2361-2367; McCleskey, E. W. (1994) Curr. Opin. Neurobiol. 4:304-312).

The transient receptor family (Trp) of calcium ion channels are thought to mediate capacitative calcium entry (CCE). CCE is the Ca²⁺ influx into cells to resupply Ca²⁺ stores depleted by the action of inositol triphosphate (IP3) and other agents in response to numerous hormones and growth factors. Trp and Trp-like were first cloned from Drosophila and have similarity to voltage gated Ca2+ channels in the S3 through S6 regions. This suggests that Trp and/or related proteins may form mammalian CCC entry channels (Zhu, X. et al. (1996) Cell 85:661-671; Boulay, G. et al. (1997) J. Biol. Chem. 272:29672-29680). Melastatin is a gene isolated in both the mouse and human, and whose expression in melanoma cells is inversely correlated with melanoma aggressiveness in vivo. The human cDNA transcript corresponds to a 1533-amino acid protein having homology to members of the Trp family. It has been proposed that the combined use of malastatin mRNA expression status and tumor thickness might allow for the determination of subgroups of patients at both low and high risk for developing metastatic disease (Duncan, L. M. et al (2001) J. Clin. Oncol. 19:568-576).

Chloride channels are necessary in endocrine secretion and in regulation of cytosolic and organelle pH. In secretory epithelial cells, Cl⁻ enters the cell across a basolateral membrane through an Na⁺, K⁺/Cl⁻ cotransporter, accumulating in the cell above its electrochemical equilibrium concentration. Secretion of Cl⁻ from the apical surface, in response to hormonal stimulation, leads to flow of Na⁺ and water into the secretory lumen. The cystic fibrosis transmembrane conductance regulator (CFTR) is a chloride channel encoded by the gene for cystic fibrosis, a common fatal genetic disorder in humans. CFTR is a member of the ABC transporter family, and is composed of two domains each consisting of six transmembrane domains followed by a nucleotide-binding site. Loss of CFTR function decreases transepithelial water secretion and, as a result, the layers of mucus that coat the respiratory tree, pancreatic ducts, and intestine are dehydrated and difficult to clear. The resulting blockage of these sites leads to pancreatic insufficiency, “meconium ileus”, and devastating “chronic obstructive pulmonary disease” (Al-Awqati, Q. et al. (1992) J. Exp. Biol. 172:245-266).

The voltage-gated chloride channels (CLC) are characterized by 10-12 transmembrane domains, as well as two small globular domains known as CBS domains. The CLC subunits probably function as homotetramers. CLC proteins are involved in regulation of cell volume, membrane potential stabilization, signal transduction, and transepithelial transport. Mutations in CLC-1, expressed predominantly in skeletal muscle, are responsible for autosomal recessive generalized myotonia and autosomal dominant myotonia congenita, while mutations in the kidney channel CLC-5 lead to kidney stones (Jentsch, T. J. (1996) Curr. Opin. Neurobiol. 6:303-310).

Ligand-gated channels open their pores when an extracellular or intracellular mediator binds to the channel. Neurotransmitter-gated channels are channels that open when a neurotransmitter binds to their extracellular domain. These channels exist in the postsynaptic membrane of nerve or muscle cells. There are two types of neurotransmitter-gated channels. Sodium channels open in response to excitatory neurotransmitters, such as acetylcholine, glutamate, and serotonin. This opening causes an influx of Na⁺ and produces the initial localized depolarization that activates the voltage-gated channels and starts the action potential. Chloride channels open in response to inhibitory neurotransmitters, such as γ-aminobutyric acid (GABA) and glycine, leading to hyperpolarization of the membrane and the subsequent generation of an action potential. Neurotransmitter-gated ion channels have four transmembrane domains and probably function as pentamers (Jentsch, supra). Amino acids in the second transmembrane domain appear to be important in determining channel permeation and selectivity (Sather, W. A. et al. (1994) Curr. Opin. Neurobiol. 4:313-323).

Ligand-gated channels can be regulated by intracellular second messengers. For example, calcium-activated K⁺ channels are gated by internal calcium ions. In nerve cells, an influx of calcium during depolarization opens K⁺ channels to modulate the magnitude of the action potential (Ishi et al., supra). The large conductance (BK) channel has been purified from brain and its subunit composition determined. The a subunit of the BK channel has seven rather than six transmembrane domains in contrast to voltage-gated K⁺ channels. The extra transmembrane domain is located at the subunit N-terminus. A 28-amino-acid stretch in the C-terminal region of the subunit (the “calcium bowl” region) contains many negatively charged residues and is thought to be the region responsible for calcium binding. The b subunit consists of two transmembrane domains connected by a glycosylated extracellular loop, with intracellular N— and C-termini (Kaczorowski, supra; Vergara, C. et al. (1998) Curr. Opin. Neurobiol. 8:321-329).

Cyclic nucleotide-gated (CNG) channels are gated by cytosolic cyclic nucleotides. The best examples of these are the cAMP-gated Na⁺ channels involved in olfaction and the cGMP-gated cation channels involved in vision. Both systems involve ligand-mediated activation of a G-protein coupled receptor which then alters the level of cyclic nucleotide within the cell. CNG channels also represent a major pathway for Ca²⁺ entry into neurons, and play roles in neuronal development and plasticity. CNG channels are tetramers containing at least two types of subunits, an a subunit which can form functional homomeric channels, and a b subunit, which modulates the channel properties. All CNG subunits have six transmembrane domains and a pore forming region between the fifth and sixth transmembrane domains, similar to voltage-gated K⁺ channels. A large C-terminal domain contains a cyclic nucleotide binding domain, while the N-terminal domain confers variation among channel subtypes (Zufall, F. et al. (1997) Curr. Opin. Neurobiol. 7:404-412).

The activity of other types of ion channel proteins may also be modulated by a variety of intracellular signalling proteins. Many channels have sites for phosphorylation by one or more protein kinases including protein kinase A, protein kinase C, tyrosine kinase, and casein kinase II, all of which regulate ion channel activity in cells. Kir channels are activated by the binding of the Gbg subunits of heterotrimeric G-proteins (Reimann, F. and F. M. Ashcroft (1999) Curr. Opin. Cell. Biol. 11:503-508). Other proteins are involved in the localization of ion channels to specific sites in the cell membrane. Such proteins include the PDZ domain proteins known as MAGUKs (membrane-associated guanylate kinases) which regulate the clustering of ion channels at neuronal synapses (Craven, S. E. and D. S. Bredt (1998) Cell 93:495-498).

Disease Correlation

The etiology of numerous human diseases and disorders can be attributed to defects in the transport of molecules across membranes. Defects in the trafficking of membrane-bound transporters and ion channels are associated with several disorders, e.g., cystic fibrosis, glucose-galactose malabsorption syndrome, hypercholesterolemia, von Gierke disease, and certain forms of diabetes mellitus. Single-gene defect diseases resulting in an inability to transport small molecules across membranes include, e.g., cystinuria, iminoglycinuria, Hartup disease, and Fanconi disease (van't Hoff, W. G. (1996) Exp. Nephrol. 4:253-262; Talente, G. M. et al. (1994) Ann. Intern. Med. 120:218-226; and Chillon, M. et al. (1995) New Engl. J. Med. 332:1475-1480).

Human diseases caused by mutations in ion channel genes include disorders of skeletal muscle, cardiac muscle, and the central nervous system. Mutations in the pore-forming subunits of sodium and chloride channels cause myotonia, a muscle disorder in which relaxation after voluntary contraction is delayed. Sodium channel myotonias have been treated with channel blockers. Mutations in muscle sodium and calcium channels cause forms of periodic paralysis, while mutations in the sarcoplasmic calcium release channel, T-tubule calcium channel, and muscle sodium channel cause malignant hyperthermia. Cardiac arrythmia disorders such as the long QT syndromes and idiopathic ventricular fibrillation are caused by mutations in potassium and sodium channels (Cooper, E. C. and L. Y. Jan (1998) Proc. Natl. Acad. Sci. USA 96:4759-4766). All four known human idiopathic epilepsy genes code for ion channel proteins (Berkovic, S. F. and I. E. Scheffer (1999) Curr. Opin. Neurology 12:177-182). Other neurological disorders such as ataxias, hemiplegic migraine and hereditary deafness can also result from mutations in ion channel genes (Jen, J. (1999) Curr. Opin. Neurobiol. 9:274-280; Cooper, supra).

Ion channels have been the target for many drug therapies. Neurotransmitter-gated channels have been targeted in therapies for treatment of insomnia, anxiety, depression, and schizophrenia. Voltage-gated channels have been targeted in therapies for arrythmia, ischemic stroke, head trauma, and neurodegenerative disease (Taylor, C. P. and L. S. Narasimhan (1997) Adv. Pharmacol. 39:47-98). Various classes of ion channels also play an important role in the perception of pain, and thus are potential targets for new analgesics. These include the vanilloid-gated ion channels, which are activated by the vanilloid capsaicin, as well as by noxious heat. Local anesthetics such as lidocaine and mexiletine which blockade voltage-gated Na+channels have been useful in the treatment of neuropathic pain (Eglen, supra).

Ion channels in the immune system have recently been suggested as targets for immunomodulation. T-cell activation depends upon calcium signaling, and a diverse set of T-cell specific ion channels has been characterized that affect this signaling process. Channel blocking agents can inhibit secretion of lymphokines, cell proliferation, and killing of target cells. A peptide antagonist of the T-cell potassium channel Kv1.3 was found to suppress delayed-type hypersensitivity and allogenic responses in pigs, validating the idea of channel blockers as safe and efficacious immunosuppressants (Cahalan, M. D. and K. G. Chandy (1997) Curr. Opin. Biotechnol. 8:749-756).

Expression Profiling

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

The discovery of new transporters and ion channels, and the polynucleotides encoding them, satisfies a need in the art by providing new compositions which are useful in the diagnosis, prevention, and treatment of transport, muscle, autoimmune/inflammatory, infectious, immune deficiencies, metabolism, reproductive, neurological, cardiovascular, eye, and cell proliferative disorders, including cancer and in the assessment of the effects of exogenous compounds on the expression of nucleic acid and amino acid sequences of transporters and ion channels.

### SUMMARY OF THE INVENTION

The invention features purified polypeptides, transporters and ion channels, referred to collectively as “TRICH” and individually as “TRICH-1,” “TRICH-2,” “TRICH-3,” “TRICH-4,” “TRICH-5,” “TRICH-6,” “TRICH-7,” “TRICH-8,” “TRICH-9,” “TRICH-10,” “TRICH-11,” “TRICH-12,” “TRICH-13,” “TRICH-14,” “TRICH-15,” “TRICH-16,” and “TRICH-17.” In one aspect, the invention provides an isolated polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. In one alternative, the invention provides an isolated polypeptide comprising the amino acid sequence of SEQ ID NO:1-17.

The invention further provides an isolated polynucleotide encoding a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. In one alternative, the polynucleotide encodes a polypeptide selected from the group consisting of SEQ ID NO:1-17. In another alternative, the polynucleotide is selected from the group consisting of SEQ ED NO:18-34.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17.

The invention further provides an isolated polynucleotide selected from the group consisting of a) a polynucleotide comprising a polynucleotide sequence selected from the group consisting of SEQ ID NO:18-34, b) a polynucleotide comprising a naturally occurring polynucleotide sequence at least 90% identical to a polynucleotide sequence selected from the group consisting of SEQ ID NO:18-34, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:18-34, b) a polynucleotide comprising a naturally occurring polynucleotide sequence at least 90% identical to a polynucleotide sequence selected from the group consisting of SEQ ID NO:18-34, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:18-34, b) a polynucleotide comprising a naturally occurring polynucleotide sequence at least 90% identical to a polynucleotide sequence selected from the group consisting of SEQ ID NO:18-34, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17, and a pharmaceutically acceptable excipient. In one embodiment, the composition comprises an amino acid sequence selected from the group consisting of SEQ ID NO:1-17. The invention additionally provides a method of treating a disease or condition associated with decreased expression of functional TRICH, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional TRICH, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional TRICH, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ED NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-17, b) a polypeptide comprising a naturally occurring amino acid sequence at least 90% identical to an amino acid sequence selected from the group consisting of SEQ ID NO:1-17, c) a biologically active fragment of a polypeptide having an amino acid sequence selected from the group consisting of SEQ ID NO:1-17, and d) an immunogenic fragment of a polypeptide having an amino acid sequence selected from the group consisting of SEQ ID NO:1-17. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:18-34, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:18-34, ii) a polynucleotide comprising a naturally occurring polynucleotide sequence at least 90% identical to a polynucleotide sequence selected from the group consisting of SEQ ID NO:18-34, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:18-34, ii) a polynucleotide comprising a naturally occurring polynucleotide sequence at least 90% identical to a polynucleotide sequence selected from the group consisting of SEQ ID NO:18-34, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog, and the PROTEOME database identification numbers and annotations of PROTEOME database homologs, for polypeptides of the invention. The probability scores for the matches between each polypeptide and its homolog(s) are also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### DEFINITIONS

“TRICH” refers to the amino acid sequences of substantially purified TRICH obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of TRICH. Agonists may include proteins, nucleic acids, carbohydrates, small molecules, or any other compound or composition which modulates the activity of TRICH either by directly interacting with TRICH or by acting on components of the biological pathway in which TRICH participates.

An “allelic variant” is an alternative form of the gene encoding TRICH. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding TRICH include those sequences with deletions, insertions, or substitutions of different nucleotides, resulting in a polypeptide the same as TRICH or a polypeptide with at least one functional characteristic of TRICH. Included within this definition are polymorphisms which may or may not be readily detectable using a particular oligonucleotide probe of the polynucleotide encoding TRICH, and improper or unexpected hybridization to allelic variants, with a locus other than the normal chromosomal locus for the polynucleotide sequence encoding TRICH. The encoded protein may also be “altered,” and may contain deletions, insertions, or substitutions of amino acid residues which produce a silent change and result in a functionally equivalent TRICH. Deliberate amino acid substitutions may be made on the basis of similarity in polarity, charge, solubility, hydrophobicity, hydrophilicity, and/or the amphipathic nature of the residues, as long as the biological or immunological activity of TRICH is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of TRICH. Antagonists may include proteins such as antibodies, nucleic acids, carbohydrates, small molecules, or any other compound or composition which modulates the activity of TRICH either by directly interacting with TRICH or by acting on components of the biological pathway in which TRICH participates.

The term “antibody” refers to intact immunoglobulin molecules as well as to fragments thereof, such as Fab, F(ab′)₂, and Fv fragments, which are capable of binding an epitopic determinant. Antibodies that bind TRICH polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic TRICH, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding TRICH or fragments of TRICH may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of TRICH or the polynucleotide encoding TRICH which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defmed sequence. Clearly these lengths are exemplary, and any length that is supported by the specification, including the Sequence Listing, tables, and figures, may be encompassed by the present embodiments.

A fragment of SEQ ID NO:18-34 comprises a region of unique polynucleotide sequence that specifically identifies SEQ ID NO:18-34, for example, as distinct from any other sequence in the genome from which the fragment was obtained. A fragment of SEQ ID NO:18-34 is useful, for example, in hybridization and amplification technologies and in analogous methods that distinguish SEQ ID NO:18-34 from related polynucleotide sequences. The precise length of a fragment of SEQ ID NO:18-34 and the region of SEQ ID NO:18-34 to which the fragment corresponds are routinely determinable by one of ordinary skill in the art based on the intended purpose for the fragment.

A fragment of SEQ ID NO:1-17 is encoded by a fragment of SEQ ID NO:18-34. A fragment of SEQ ID NO:1-17 comprises a region of unique amino acid sequence that specifically identifies SEQ ID NO:1-17. For example, a fragment of SEQ ID NO:1-17 is useful as an immunogenic peptide for the development of antibodies that specifically recognize SEQ ID NO:1-17. The precise length of a fragment of SEQ ID NO:1-17 and the region of SEQ ID NO:1-17 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (T.) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of TRICH which is capable of eliciting an immune response when introduced into a living organism, for example, a mammal. The term “immunogenic fragment” also includes any polypeptide or oligopeptide fragment of TRICH which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of TRICH. For example, modulation may cause an increase or a decrease in protein activity, binding characteristics, or any other biological, functional, or immunological properties of TRICH.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an TRICH may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of TRICH.

“Probe” refers to nucleic acid sequences encoding TRICH, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing TRICH, nucleic acids encoding TRICH, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In one alternative, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human transporters and ion channels (TRICH), the polynucleotides encoding TRICH, and the use of these compositions for the diagnosis, treatment, or prevention of transport, muscle, autoimmune/inflammatory, infectious, immune deficiencies, metabolism, reproductive, neurological, cardiovascular, eye, and cell proliferative disorders, including cancer.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTlFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are transporters and ion channels. For example, SEQ ID NO:2 is 81% identical, from residue M1 to residue G1484, to a putative E1-E2 ATPase from mouse (GenBank ID g6457270) as determined by the Basic Local Alignment Search Tool (BLAST). (See Table 2.) The BLAST probability score is 0.0, which indicates the probability of obtaining the observed polypeptide sequence alignment by chance. Data from BLIMPS, MOTIFS, and PROFILESCAN analyses provide further corroborative evidence that SEQ ID NO:2 is an E1-E2 ATPase. (See Table 3.) In another example, SEQ ID NO:3 is 83% identical, from residue A182 to residue I811, to mouse fatty acid transport protein 3 (GenBank ID g3335567) with a BLAST probability score of 1.9e-285. Data from BLIMPS, MOTIFS, and PROFILESCAN analyses provide further corroborative evidence that SEQ ID NO:3 is a fatty acid transport protein. In a futher example, SEQ ID NO:5 is 30% identical, from residue 1876 to residue Q1559, 36% identical, from residue K1266 to residue E1482, 48% identical, from residue E594 to residue 1677, and 32% identical, from residue 1595 to residue Q766, to human ATP-binding cassette transporter 1 (GenBank ID g9755159) with a BLAST probability score of 2.1e-111. SEQ ID NO:5 also contains an ABC transporter domain as determined by searching for statistically significant matches in the hidden Markov model (HMM)-based PFAM database of conserved protein family domains. (See Table 3.) Data from additional BLAST analyses provide further corroborative evidence that SEQ ID NO:5 belongs to the ABC Transporters Family. In another example, SEQ ID NO:9 is 94% identical, from residue L9 to residue D341 and is 87% identical, from residue N323 to residue C1177, to rabbit RING-finger binding protein (GenBank ID g7715417) with a BLAST probability score of 0.0. SEQ ID NO:9 also contains E1-E2 ATPase and haloacid dehalogenase-like hydrolase domains as determined by searching for statistically significant matches in the hidden Markov model (HMM)-based PFAM database of conserved protein family domains. Data from BLIMPS, MOTIFS, and PROFILESCAN analyses provide further corroborative evidence that SEQ ID NO:9 is an E1-E2 ATPase. In yet another example, SEQ ID NO:14 is 72% identical, from residue T78 to residue E256, to human voltage-dependent anion channel isoform 2 (GenBank ID g5114261) with a BLAST probability score of 2.1e-86. SEQ ID NO:14 also contains an eukaryotic porin domain as determined by searching for statistically significant matches in the hidden Markov model (HMM)-based PFAM database of conserved protein family domains. Data from BLIMPS and PROFILESCAN analyses provide further corroborative evidence that SEQ ID NO:14 is a voltage-dependent anion channel. In another example, SEQ ID NO:16 is 93% identical, from residue M1 to residue S1095, to mouse E1-E2 ATPase (transbilayer amphipath transporter) (GenBank ID g6435130) with a BLAST probability score of 0.0. SEQ ID NO:16 also contains E1-E2 ATPase domains as determined by searching for statistically significant matches in the hidden Markov model (HMM)-based PFAM database of conserved protein family domains. Data from BLIMPS, MOTIFS, and PROFILESCAN analyses provide further corroborative evidence that SEQ ID NO:16 is a P-type ATPase. SEQ ID NO:1, SEQ ID NO:4, SEQ ID NO:6-8, SEQ ID NO:10-13, SEQ ID NO:15, and SEQ ID NO:17 were analyzed and annotated in a similar manner. The algorithms and parameters for the analysis of SEQ ID NO:1-17 are described in Table 7.

As shown in Table 4, the full length polynucleotide sequences of the present invention were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide sequences of the invention, and of fragments of the polynucleotide sequences which are useful, for example, in hybridization or amplification technologies that identify SEQ ID NO:18-34 or that distinguish between SEQ ID NO:18-34 and related polynucleotide sequences.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotide sequences. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N₁_{_{—}}N₂_{_{—}}YYYYY_N₃_{_{—}}N₄ represents a “stitched” sequence in which XXXXXX is the identification number of the cluster of sequences to which the algorithm was applied, and YYYYY is the number of the prediction generated by the algorithm, and N_{1,2,3, . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences |
| ENST | using, for example, GENSCAN (Stanford |
| | University, CA, USA) or FGENES |
| | (Computer Genomics Group, The Sanger |
| | Centre, Cambridge, UK). |
| GBI | Hand-edited analysis of genomic sequences. |
| FL | Stitched or stretched genomic sequences (see |
| | Example V). |
| INCY | Full length transcript and exon prediction from |
| | mapping of EST sequences to the genome. |
| | Genomic location and EST composition |
| | data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses TRICH variants. A preferred TRICH variant is one which has at least about 80%, or alternatively at least about 90%, or even at least about 95% amino acid sequence identity to the TRICH amino acid sequence, and which contains at least one functional or structural characteristic of TRICH.

The invention also encompasses polynucleotides which encode TRICH. In a particular embodiment, the invention encompasses a polynucleotide sequence comprising a sequence selected from the group consisting of SEQ ID NO:18-34, which encodes TRICH. The polynucleotide sequences of SEQ ID NO:18-34, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding TRICH. In particular, such a variant polynucleotide sequence will have at least about 70%, or alternatively at least about 85%, or even at least about 95% polynucleotide sequence identity to the polynucleotide sequence encoding TRICH. A particular aspect of the invention encompasses a variant of a polynucleotide sequence comprising a sequence selected from the group consisting of SEQ ID NO:18-34 which has at least about 70%, or alternatively at least about 85%, or even at least about 95% polynucleotide sequence identity to a nucleic acid sequence selected from the group consisting of SEQ ID NO:18-34. Any one of the polynucleotide variants described above can encode an amino acid sequence which contains at least one functional or structural characteristic of TRICH.

In addition, or in the alternative, a polynucleotide variant of the invention is a splice variant of a polynucleotide sequence encoding TRICH. A splice variant may have portions which have significant sequence identity to the polynucleotide sequence encoding TRICH, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to the polynucleotide sequence encoding TRICH over its entire length; however, portions of the splice variant will have at least about 70%, or alternatively at least about 85%, or alternatively at least about 95%, or alternatively 100% polynucleotide sequence identity to portions of the polynucleotide sequence encoding TRICH. For example, a polynucleotide comprising a sequence of SEQ ID NO:20 is a splice variant of a polynucleotide comprising a sequence of SEQ ID NO:34. Any one of the splice variants described above can encode an amino acid sequence which contains at least one functional or structural characteristic of TRICH.

It will be appreciated by those skilled in the art that as a result of the degeneracy of the genetic code, a multitude of polynucleotide sequences encoding TRICH, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring TRICH, and all such variations are to be considered as being specifically disclosed.

Although nucleotide sequences which encode TRICH and its variants are generally capable of hybridizing to the nucleotide sequence of the naturally occurring TRICH under appropriately selected conditions of stringency, it may be advantageous to produce nucleotide sequences encoding TRICH or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding TRICH and its derivatives without altering the encoded amino acid sequences include the production of RNA transcripts having more desirable properties, such as a greater half-life, than transcripts produced from the naturally occurring sequence.

The invention also encompasses production of DNA sequences which encode TRICH and TRICH derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding TRICH or any fragment thereof.

Also encompassed by the invention are polynucleotide sequences that are capable of hybridizing to the claimed polynucleotide sequences, and, in particular, to those shown in SEQ ID NO:18-34 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding TRICH may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode TRICH may be cloned in recombinant DNA molecules that direct expression of TRICH, or fragments or functional equivalents thereof, in appropriate host cells. Due to the inherent degeneracy of the genetic code, other DNA sequences which encode substantially the same or a functionally equivalent amino acid sequence may be produced and used to express TRICH.

The nucleotide sequences of the present invention can be engineered using methods generally known in the art in order to alter TRICH-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of TRICH, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding TRICH may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, TRICH itself or a fragment thereof may be synthesized using chemical methods. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of TRICH, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active TRICH, the nucleotide sequences encoding TRICH or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding TRICH. Such elements may vary in their strength and specificity. Specific initiation signals may also be used to achieve more efficient translation of sequences encoding TRICH. Such signals include the ATG initiation codon and adjacent sequences, e.g. the Kozak sequence. In cases where sequences encoding TRICH and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding TRICH and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding TRICH. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding TRICH. For example, routine cloning, subcloning, and propagation of polynucleotide sequences encoding TRICH can be achieved using a multifunctional E. coli vector such as PBLUESCRIPT (Stratagene, La Jolla Calif.) or PSPORT1 plasmid (Life Technologies). Ligation of sequences encoding TRICH into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of TRICH are needed, e.g. for the production of antibodies, vectors which direct high level expression of TRICH may be used. For example, vectors containing the strong, inducible SP6 or T7 bacteriophage promoter may be used.

Yeast expression systems may be used for production of TRICH. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of TRICH. Transcription of sequences encoding TRICH may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding TRICH may be ligated into an adenovirus transcription/translation complex consisting of the late promoter and tripartite leader sequence. Insertion in a non-essential E1 or E3 region of the viral genome may be used to obtain infective virus which expresses TRICH in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of TRICH in cell lines is preferred. For example, sequences encoding TRICH can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding TRICH is inserted within a marker gene sequence, transformed cells containing sequences encoding TRICH can be identified by the absence of marker gene function. Alternatively, a marker gene can be placed in tandem with a sequence encoding TRICH under the control of a single promoter. Expression of the marker gene in response to induction or selection usually indicates expression of the tandem gene as well.

In general, host cells that contain the nucleic acid sequence encoding TRICH and that express TRICH may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of TRICH using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on TRICH is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding TRICH include oligolabeling, nick translation, end-labeling, or PCR amplification using a labeled nucleotide. Alternatively, the sequences encoding TRICH, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding TRICH may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode TRICH may be designed to contain signal sequences which direct secretion of TRICH through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

In another embodiment of the invention, natural, modified, or recombinant nucleic acid sequences encoding TRICH may be ligated to a heterologous sequence resulting in translation of a fusion protein in any of the aforementioned host systems. For example, a chimeric TRICH protein containing a heterologous moiety that can be recognized by a commercially available antibody may facilitate the screening of peptide libraries for inhibitors of TRICH activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the TRICH encoding sequence and the heterologous protein sequence, so that TRICH may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled TRICH may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

TRICH of the present invention or fragments thereof may be used to screen for compounds that specifically bind to TRICH. At least one and up to a plurality of test compounds may be screened for specific binding to TRICH. Examples of test compounds include antibodies, oligonucleotides, proteins (e.g., receptors), or small molecules.

In one embodiment, the compound thus identified is closely related to the natural ligand of TRICH, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which TRICH binds, or to at least a fragment of the receptor, e.g., the ligand binding site. In either case, the compound can be rationally designed using known techniques. In one embodiment, screening for these compounds involves producing appropriate cells which express TRICH, either as a secreted protein or on the cell membrane. Preferred cells include cells from mammals, yeast, Drosophila, or E. coli. Cells expressing TRICH or cell membrane fractions which contain TRICH are then contacted with a test compound and binding, stimulation, or inhibition of activity of either TRICH or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with TRICH, either in solution or affixed to a solid support, and detecting the binding of TRICH to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

TRICH of the present invention or fragments thereof may be used to screen for compounds that modulate the activity of TRICH. Such compounds may include agonists, antagonists, or partial or inverse agonists. In one embodiment, an assay is performed under conditions permissive for TRICH activity, wherein TRICH is combined with at least one test compound, and the activity of TRICH in the presence of a test compound is compared with the activity of TRICH in the absence of the test compound. A change in the activity of TRICH in the presence of the test compound is indicative of a compound that modulates the activity of TRICH. Alternatively, a test compound is combined with an in vitro or cell-free system comprising TRICH under conditions suitable for TRICH activity, and the assay is performed. In either of these assays, a test compound which modulates the activity of TRICH may do so indirectly and need not come in direct contact with the test compound. At least one and up to a plurality of test compounds may be screened.

In another embodiment, polynucleotides encoding TRICH or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding TRICH may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding TRICH can also be used to create “knockin” humanized animals (pigs) or transgenic animals (mice or rats) to model human disease. With knockin technology, a region of a polynucleotide encoding TRICH is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress TRICH, e.g., by secreting TRICH in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of TRICH and transporters and ion channels. In addition, examples of tissues expressing TRICH are tumorous colon tissue, primary human breast epithelial cells (HMEC), B cell lymphoblast cells, peripheral blood mononuclear cells (PBMCs), aortic endothelial cells (HAECs), breast tumor cells, Jurkat T-cell leukemia cells, and cells derived from the endothelium of the human umbilical vein (ECV304) cell line. Further examples of tissues expressing TRICH can also be found in Table 6. Therefore, TRICH appears to play a role in transport, muscle, autoimmune/inflammatory, infectious, immune deficiencies, metabolism, reproductive, neurological, cardiovascular, eye, and cell proliferative disorders, including cancer. In the treatment of disorders associated with increased TRICH expression or activity, it is desirable to decrease the expression or activity of TRICH. In the treatment of disorders associated with decreased TRICH expression or activity, it is desirable to increase the expression or activity of TRICH.

Therefore, in one embodiment, TRICH or a fragment or derivative thereof may be administered to a subject to treat or prevent a disorder associated with decreased expression or activity of TRICH. Examples of such disorders include, but are not limited to, a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulmonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease; a muscle disorder such as cardiomyopathy, myocarditis, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, ethanol myopathy, angina, anaphylactic shock, arrhythmias, asthma, cardiovascular shock, Cushing's syndrome, hypertension, hypoglycemia, myocardial infarction, migraine, pheochromocytoma, and myopathies including encephalopathy, epilepsy, Kearns-Sayre syndrome, lactic acidosis, myoclonic disorder, ophthalmoplegia, and acid maltase deficiency (AMD, also known as Pompe's disease); an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, and trauma; an infectious disorder such as a viral infection, e.g., caused by an adenovirus (acute respiratory disease, pneumonia), an arenavirus (lymphocytic choriomeningitis), a bunyavirus (Hantavirus), a coronavirus (pneumonia, chronic bronchitis), a hepadnavirus (hepatitis), a herpesvirus (herpes simplex virus, varicella-zoster virus, Epstein-Barr virus, cytomegalovirus), a flavivirus (yellow fever), an orthomyxovirus (influenza), a papillomavirus (cancer), a paramyxovirus (measles, mumps), a picornovirus (rhinovirus, poliovirus, coxsackie-virus), a polyomavirus (BK virus, JC virus), a poxvirus (smallpox), a reovirus (Colorado tick fever), a retrovirus (human immunodeficiency virus, human T lymphotropic virus), a rhabdovirus (rabies), a rotavirus (gastroenteritis), and a togavirus (encephalitis, rubella), and a bacterial infection, a fungal infection, a parasitic infection, a protozoal infection, and a helninthic infection; an immune deficiency, such as X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a disorder of metabolism such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, a lipid myopathy, a lipodystrophy, a lysosomal storage disease, mannosidosis, neuraminidase deficiency, obesity, pentosuria phenylketonuria, pseudovitamin D-deficiency rickets; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis, fibrocystic breast disease, and galactorrhea, disruptions of spermatogenesis, abnormal sperm physiology, benign prostatic hyperplasia, prostatitis, Peyronie's disease, impotence, carcinoma of the male breast, and gynecomastia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, anresia, catatonia, diabetic neuropathy, tardive dyskinesia, dystonias, paranoid psychoses, postherpetic neuralgia, and Tourette's disorder; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an eye disorder such as ocular hypertension and glaucoma; a disorder of cell proliferation such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and a cancer, including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a vector capable of expressing TRICH or a fragment or derivative thereof may be administered to a subject to treat or prevent a disorder associated with decreased expression or activity of TRICH including, but not limited to, those described above.

In a further embodiment, a composition comprising a substantially purified TRICH in conjunction with a suitable pharmaceutical carrier may be administered to a subject to treat or prevent a disorder associated with decreased expression or activity of TRICH including, but not limited to, those provided above.

In still another embodiment, an agonist which modulates the activity of TRICH may be administered to a subject to treat or prevent a disorder associated with decreased expression or activity of TRICH including, but not limited to, those listed above.

In a further embodiment, an antagonist of TRICH may be administered to a subject to treat or prevent a disorder associated with increased expression or activity of TRICH. Examples of such disorders include, but are not limited to, those transport, muscle, autoimmune/inflammatory, infectious, immune deficiencies, metabolism, reproductive, neurological, cardiovascular, eye, and cell proliferative disorders, including cancer described above. In one aspect, an antibody which specifically binds TRICH may be used directly as an antagonist or indirectly as a targeting or delivery mechanism for bringing a pharmaceutical agent to cells or tissues which express TRICH.

In an additional embodiment, a vector expressing the complement of the polynucleotide encoding TRICH may be administered to a subject to treat or prevent a disorder associated with increased expression or activity of TRICH including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of TRICH may be produced using methods which are generally known in the art. In particular, purified TRICH may be used to produce antibodies or to screen libraries of pharmaceutical agents to identify those which specifically bind TRICH. Antibodies to TRICH may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with TRICH or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to TRICH have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of TRICH amino acids may be fused with those of another protein, such as KLH, and antibodies to the chimeric molecule may be produced.

Monoclonal antibodies to TRICH may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce TRICH-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for TRICH may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between TRICH and its specific antibody. A two-site, monoclonal-based immunoassay utilizing monoclonal antibodies reactive to two non-interfering TRICH epitopes is generally used, but a competitive binding assay may also be employed (Pound, supra).

Various methods such as Scatchard analysis in conjunction with radioimmunoassay techniques may be used to assess the affinity of antibodies for TRICH. Affinity is expressed as an association constant, Kₐ, which is defined as the molar concentration of TRICH-antibody complex divided by the molar concentrations of free antigen and free antibody under equilibrium conditions. The Kₐ determined for a preparation of polyclonal antibodies, which are heterogeneous in their affinities for multiple TRICH epitopes, represents the average affinity, or avidity, of the antibodies for TRICH. The Kₐ determined for a preparation of monoclonal antibodies, which are monospecific for a particular TRICH epitope, represents a true measure of affinity. High-affinity antibody preparations with Kₐ ranging from about 10⁹ to 10¹² L/mole are preferred for use in immunoassays in which the TRICH-antibody complex must withstand rigorous manipulations. Low-affinity antibody preparations with Kₐ ranging from about 10⁶ to 10⁷ L/mole are preferred for use in immunopurification and similar procedures which ultimately require dissociation of TRICH, preferably in active form, from the antibody (Catty, D. (1988) Antibodies, Volume I: A Practical Approach, IRL Press, Washington DC; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of TRICH-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding TRICH, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding TRICH. Such technology is well known in the art, and antisense oligonucleotides or larger fragments can be designed from various locations along the coding or control regions of sequences encoding TRICH. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding TRICH may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480, Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in TRICH expression or regulation causes disease, the expression of TRICH from an appropriate population of transduced cells may alleviate the clinical manifestations caused by the genetic deficiency.

In a further embodiment of the invention, diseases or disorders caused by deficiencies in TRICH are treated by constructing mammalian expression vectors encoding TRICH and introducing these vectors by mechanical means into TRICH-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Recipon (1998) Curr. Opin. Biotechnol. 9:445-450).

Expression vectors that may be effective for the expression of TRICH include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). TRICH may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding TRICH from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to TRICH expression are treated by constructing a retrovirus vector consisting of (i) the polynucleotide encoding TRICH under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding TRICH to cells which have one or more genetic abnormalities with respect to the expression of TRICH. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding TRICH to target cells which have one or more genetic abnormalities with respect to the expression of TRICH. The use of herpes simplex virus (HSV)-based vectors may be especially valuable for introducing TRICH to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding TRICH to target cells. The biology of the prototypic alphavirus, Semliki Forest, Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for TRICH into the alphavirus genome in place of the capsid-coding region results in the production of a large number of TRICH-coding RNAs and the synthesis of high levels of TRICH in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of TRICH into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding TRICH.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding TRICH. Such DNA sequences may be incorporated into a wide variety of vectors with suitable RNA polymerase promoters such as T7 or SP6. Alternatively, These cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding TRICH. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased TRICH expression or activity, a compound which specifically inhibits expression of the polynucleotide encoding TRICH may be therapeutically useful, and in the treatment of disorders associated with decreased TRICH expression or activity, a compound which specifically promotes expression of the polynucleotide encoding TRICH may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding TRICH is exposed to at least one test compound thus obtained. The sample may comprise, for example, an intact or permeabilized cell, or an in vitro cell-free or reconstituted biochemical system. Alterations in the expression of a polynucleotide encoding TRICH are assayed by any method commonly known in the art. Typically, the expression of a specific nucleotide is detected by hybridization with a probe having a nucleotide sequence complementary to the sequence of the polynucleotide encoding TRICH. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of TRICH, antibodies to TRICH, and mimetics, agonists, antagonists, or inhibitors of TRICH.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising TRICH or fragments thereof. For example, liposome preparations containing a cell-impermeable macromolecule may promote cell fusion and intracellular delivery of the macromolecule. Alternatively, TRICH or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example TRICH or fragments thereof, antibodies of TRICH, and agonists, antagonists or inhibitors of TRICH, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind TRICH may be used for the diagnosis of disorders characterized by expression of TRICH, or in assays to monitor patients being treated with TRICH or agonists, antagonists, or inhibitors of TRICH. Antibodies useful for diagnostic purposes may be prepared in the same manner as described above for therapeutics. Diagnostic assays for TRICH include methods which utilize the antibody and a label to detect TRICH in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring TRICH, including ELISAs, RIAs, and FACS, are known in the art and provide a basis for diagnosing altered or abnormal levels of TRICH expression. Normal or standard values for TRICH expression are established by combining body fluids or cell extracts taken from normal mammalian subjects, for example, human subjects, with antibodies to TRICH under conditions suitable for complex formation. The amount of standard complex formation may be quantitated by various methods, such as photometric means. Quantities of TRICH expressed in subject, control, and disease samples from biopsied tissues are compared with the standard values. Deviation between standard and subject values establishes the parameters for diagnosing disease.

In another embodiment of the invention, the polynucleotides encoding TRICH may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of TRICH may be correlated with disease. The diagnostic assay-may be used to determine absence, presence, and excess expression of TRICH, and to monitor regulation of TRICH levels during therapeutic intervention.

In one aspect, hybridization with PCR probes which are capable of detecting polynucleotide sequences, including genomic sequences, encoding TRICH or closely related molecules may be used to identify nucleic acid sequences which encode TRICH. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding TRICH, allelic variants, or related sequences.

Probes may also be used for the detection of related sequences, and may have at least 50% sequence identity to any of the TRICH encoding sequences. The hybridization probes of the subject invention may be DNA or RNA and may be derived from the sequence of SEQ ID NO:18-34 or from genomic sequences including promoters, enhancers, and introns of the TRICH gene.

Means for producing specific hybridization probes for DNAs encoding TRICH include the cloning of polynucleotide sequences encoding TRICH or TRICH derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding TRICH may be used for the diagnosis of disorders associated with expression of TRICH. Examples of such disorders include, but are not limited to, a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulmonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease; a muscle disorder such as cardiomyopathy, myocarditis, Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, ethanol myopathy, angina, anaphylactic shock, arrhythmias, asthma, cardiovascular shock, Cushing's syndrome, hypertension, hypoglycemia, myocardial infarction, migraine, pheochromocytoma, and myopathies including encephalopathy, epilepsy, Kearns-Sayre syndrome, lactic acidosis, myoclonic disorder, ophthalmoplegia, and acid maltase deficiency (AMD, also known as Pompe's disease); an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, and trauma; an infectious disorder such as a viral infection, e.g., caused by an adenovirus (acute respiratory disease, pneumonia), an arenavirus (lymphocytic choriomeningitis), a bunyavirus (Hantavirus), a coronavirus (pneumonia, chronic bronchitis), a hepadnavirus (hepatitis), a herpesvirus (herpes simplex virus, varicella-zoster virus, Epstein-Barr virus, cytomegalovirus), a flavivirus (yellow fever), an orthomyxovirus (influenza), a papillomavirus (cancer), a paramyxovirus (measles, mumps), a picornovirus (rhinovirus, poliovirus, coxsackie-virus), a polyomavirus (BK virus, JC virus), a poxvirus (smallpox), a reovirus (Colorado tick fever), a retrovirus (human immunodeficiency virus, human T lymphotropic virus), a rhabdovirus (rabies), a rotavirus (gastroenteritis), and a togavirus (encephalitis, rubella), and a bacterial infection, a fungal infection, a parasitic infection, a protozoal infection, and a helminthic infection; an immune deficiency, such as X-linked agammaglobinemia of Bruton, common variable immunodeficiency (CVI), DiGeorge's syndrome (thymic hypoplasia), thymic dysplasia, isolated IgA deficiency, severe combined immunodeficiency disease (SCID), immunodeficiency with thrombocytopenia and eczema (Wiskott-Aldrich syndrome), Chediak-Higashi syndrome, chronic granulomatous diseases, hereditary angioneurotic edema, and immunodeficiency associated with Cushing's disease; a disorder of metabolism such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, a lipid myopathy, a lipodystrophy, a lysosomal storage disease, mannosidosis, neuraminidase deficiency, obesity, pentosuria phenylketonuria, pseudovitamin D-deficiency rickets; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis, fibrocystic breast disease, and galactorrhea, disruptions of spermatogenesis, abnormal sperm physiology, benign prostatic hyperplasia, prostatitis, Peyronie's disease, impotence, carcinoma of the male breast, and gynecomastia; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease; prion diseases including kuru, Creutzfeldt-Jakob disease, and Gerstmann-Straussler-Scheinker syndrome; fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis; inherited, metabolic, endocrine, and toxic myopathies; myasthenia gravis, periodic paralysis; mental disorders including mood, anxiety, and schizophrenic disorders; seasonal affective disorder (SAD); akathesia, amnesia, catatonia, diabetic neuropathy, tardive dyskinesia, dystonias, paranoid psychoses, postherpetic neuralgia, and Tourette's disorder; a cardiovascular disorder, such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; an eye disorder such as ocular hypertension and glaucoma; a disorder of cell proliferation such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia; and a cancer, including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. The polynucleotide sequences encoding TRICH may be used in Southern or northern analysis, dot blot, or other membrane-based technologies; in PCR technologies; in dipstick, pin, and multiformat ELISA-like assays; and in microarrays utilizing fluids or tissues from patients to detect altered TRICH expression. Such qualitative or quantitative methods are well known in the art.

In a particular aspect, the nucleotide sequences encoding TRICH may be useful in assays that detect the presence of associated disorders, particularly those mentioned above. The nucleotide sequences encoding TRICH may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding TRICH in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of TRICH, a normal or standard profile for expression is established. This may be accomplished by combining body fluids or cell extracts taken from normal subjects, either animal or human, with a sequence, or a fragment thereof, encoding TRICH, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding TRICH may involve the use of PCR. These oligomers may be chemically synthesized, generated enzymatically, or produced in vitro. Oligomers will preferably contain a fragment of a polynucleotide encoding TRICH, or a fragment of a polynucleotide complementary to the polynucleotide encoding TRICH, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding TRICH may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding TRICH are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of TRICH include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, TRICH, fragments of TRICH, or antibodies specific for TRICH may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels ii an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for TRICH to quantify the levels of TRICH expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding TRICH may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Uhrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding TRICH on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, TRICH, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between TRICH and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with TRICH, or fragments thereof, and washed. Bound TRICH is then detected by methods well known in the art. Purified TRICH can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding TRICH specifically compete with a test compound for binding TRICH. In this manner, antibodies can be used to detect the presence of any peptide which shares one or more antigenic determinants with TRICH.

In additional embodiments, the nucleotide sequences which encode TRICH may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, including U.S. Ser. No. 60/283,440, U.S. Ser. No. 60/285,592,U.S. Ser. No. 60/287,263, U.S. Ser. No. 60/288,666, U.S. Ser. No. 60/292,042, U.S. Ser. No. 60/293,724, and U.S. Ser. No. 60/351,107, are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyaniate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH15α, DH10B, or ElectroMAX DH10B from Life Technologies.

II. Isolation of cDNA Clones

Plasmids obtained as described in Example 1 were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:18-34. Fragments from about 20 to about 4000 nucleotides which are useful in hybridization and amplification technologies are described in Table 4, column 2.

IV. Identification and Editing of Coding Sequences from Genomic DNA

Putative transporters and ion channels were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode transporters and ion channels, the encoded polypeptides were analyzed by querying against PFAM models for transporters and ion channels. Potential transporters and ion channels were also identified by homology to Incyte cDNA sequences that had been annotated as transporters and ion channels. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example Ell. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of TRICH Encoding Polynucleotides

The sequences which were used to assemble SEQ ID NO:18-34 were compared with sequences from the Incyte LIFESEQ database and public domain databases using BLAST and other implementations of the Smith-Waterman algorithm. Sequences from these databases that matched SEQ ID NO:18-34 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score ⨯ Percent Identity}{5 ⨯ minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding TRICH are analyzed with respect to the tissue sources from wtich they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding TRICH. cDNA sequences and cDNA library/tissue information are found in the LIFESEQ GOLD database (Incyte Genomics, Palo Alto Calif.).

VIII. Extension of TRICH Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Identification of Single Nucleotide Polymorphisms in TRICH Encoding Polynucleotides

Common DNA sequence variants known as single nucleotide polymorphisms (SNPs) were identified in SEQ ID NO:18-34 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:18-34 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc.,.Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

Expression

Preparation of Progressively Senescent, Presenescent, and Senescent Cells

HMECs, which are a primary human breast epithelial cell line isolated from a normal donor, were grown in Mammary Epithelial Cell Growth Medium (Clonetics, Walkersville Md.) supplemented with 10 ng/ml human recombinant epidermal growth factor, 5 mg/ml insulin, 0.5 mg/ml hydrocortisone, 50 mg/ml gentamicin, 50 ng/ml amphotericin-B, and 0.5 mg/ml bovine pituitary extract. Cells were grown to 70-80% confluence prior to harvesting. About 1×10⁷ cells were harvested at passage 8 (progenitor cells), passages 10 and 12 (progressively senescent cells), passage 14 (presenescent cells), and passage 15 (senescent cells).

Isolation and Labeling of Sample cDNAs

Cells were harvested and lysed in 1 ml of TRIZOL reagent (5×10⁶ cells/ml; Life Technologies). The lysates were vortexed thoroughly and incubated at room temperature for 2-3 minutes and extracted with 0.5 ml chloroform. The extract was mixed, incubated at room temperature for 5 minutes, and centrifuged at 16,000×g for 15 minutes at 4° C. The aqueous layer was collected and an equal volume of isopropanol was added. Samples were mixed, incubated at room temperature for 10 minutes, and centrifuged at 16,000×g for 20 minutes at 4° C. The supernatant was removed and the RNA pellet was washed with 1 ml of 70% ethanol, centrifuged at 16,000×g at 4° C., and resuspended in RNase-free water. The concentration of the RNA was determined by measuring the optical density at 260 nm.

Poly(A) RNA was prepared using an OLIGOTEX mRNA kit (QIAGEN) with the following modifications: OLIGOTEX beads were washed in tubes instead of on spin columns, resuspended in elution buffer, and then loaded onto spin columns to recover mRNA. To obtain maximum yield, the mRNA was eluted twice.

Each poly(A) RNA sample was reverse transcribed using MMLV reverse-transcriptase, 0.05 pg/μl oligo-d(T) primer (21mer), 1× first strand buffer, 0.03 units/μl RNase inhibitor, 500 μM dATP, 500 μM dGTP, 500 μM dTTP, 40 μM dCTP, and 40 μM either dCTP-Cy3 or dCTP-Cy5 (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

cDNAs were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml sodium acetate, and 300 ml of 100% ethanol. The cDNAs were then dried to completion using a SpeedVAC system (Savant Instruments, Holbrook N.Y.) and resuspended in 14 μl 5×SSC, 0.2% SDS.

For example, using the microarray procedures described above it was determined that there was a greater than two-fold decrease in expression of transcribed messenger RNA which corresponds to the amino acid sequence of SEQ ID NO:5 and the polynucleotide sequence of SEQ ID NO:22 in senescent cells at passage 15 relative to expression of such RNA in progenitor cells.

Acute T cell leukemia cell lines were treated with combinations of graded doses of PMA and Ionomycin and collected at one hour time points. B cell lymphoblast cell lines derived from the peripheral blood of a male donor were treated with E. coli lipopolysacharrides for 0.5, 1, 2, 4, and 8 hours. Peripheral blood mononuclear cells (PBMCs) were isolated from the blood of four healthy donors and were treated with Staphylococcal endotoxins in the presence or absence of IL-4 for 2, 4, 24, and 72 hours. Aortic endothelial cells (HAECs) were grown to 85% confluency and were then treated with TNF-a for 1, 2, 4, 6, 8, 10, 24, and 48 hours.

In another example, SEQ ID NO:25 and SEQ ID NO:26 showed differential expression in inflammatory responses as determined by microarray analysis. The expression of SEQ ID NO:25 was decreased by at least two fold in an acute T cell leukemia cell line treated with PMA (a broad activator of protein kinase C-dependent pathways) and with Inomycin (a calcium ionophore that permits the entry of calcium in the cell). The expression of SEQ ID NO:26 was decreased by at least two fold in a B cell lymphoblast cell line treated with lipopolysaccharides, was decreased by at least 2 fold in peripheral blood mononuclear cells (12% B lymphocytes, 40% T lymphocytes, 20% NK cells, 25% monocytes, and 3% various cells that include dendritic and progenitor cells) treated with Staphylococcal endotoxins in the presence or absence of Interleukin-4 (IL-4), and was decreased by at least two fold in aortic endothelial cells treated with TNF-a, a pleotropic cytokine which mediates inflammatory responses through signal transduction pathways. Therefore, SEQ ID NO:25 and SEQ ID NO:26 are useful in diagnostic assays for inflammatory responses.

In yet another example, SEQ ID NO:26 and SEQ ID NO:29 showed differential expression in breast tumor cell lines versus normal breast epithelial cells as determined by microarray analysis. The expression of SEQ ID NO:26 was decreased by at least two fold in breast tumor cell lines which were harvested from donors with early stages of tumor progression and was not differentially expressed in the cell lines which were harvested from donors with late stages of tumor progression. Therefore, SEQ ID NO:26 is useful in diagnostic assays for early detection of breast cancer. The expression of SEQ ID NO:29 was decreased by at least two fold in breast tumor cell lines that were harvested from donors with both early and late stages of tumor progression and malignant transformation. Therefore, SEQ ID NO:29 is useful in diagnostic assays for both early and late stages of breast cancer.

Normal and various stages of tumorigenic breast cell lines were purchased from American Type Culture Collection (ATCC), (Manassas, Va.).

XII. Complementary Polynucleotides

Sequences complementary to the TRICH-encoding sequences, or any parts thereof, are used to detect, decrease, or inhibit expression of naturally occurring TRICH. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of TRICH. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the TRICH-encoding transcript.

XIII. Expression of TRICH

Expression and purification of TRICH is achieved using bacterial or virus-based expression systems. For expression of TRICH in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express TRICH upon induction with isopropyl beta-D-thiogalactopyranoside (IPTG). Expression of TRICH in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding TRICH by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, TRICH is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from TRICH at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified TRICH obtained by these methods can be used directly in the assays shown in Examples XVII, XVIII, and XIX, where applicable.

XIV. Functional Assays

TRICH function is assessed by expressing the sequences encoding TRICH at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of TRICH on gene expression can be assessed using highly purified populations of cells transfected with sequences encoding TRICH and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding TRICH and other genes of interest can be analyzed by northern analysis or microarray techniques.

XV. Production of TRICH Specific Antibodies

TRICH substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the TRICH amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.) Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-TRICH activity by, for example, binding the peptide or TRICH to a substrate, blocking with 1% BSA, reacting with rabbit antisera, washing, and reacting with radio-iodinated goat anti-rabbit IgG.

XVI. Purification of Naturally Occurring TRICH Using Specific Antibodies

Naturally occurring or recombinant TRICH is substantially purified by immunoaffinity chromatography using antibodies specific for TRICH. An immunoaffinity column is constructed by covalently coupling anti-TRICH antibody to an activated chromatographic resin, such as CNBr-activated SEPHAROSE (Amersham Pharmacia Biotech). After the coupling, the resin is blocked and washed according to the manufacturer's instructions.

Media containing TRICH are passed over the immunoaffinity column, and the column is washed under conditions that allow the preferential absorbance of TRICH (e.g., high ionic strength buffers in the presence of detergent). The column is eluted under conditions that disrupt antibody/TRICH binding (e.g., a buffer of pH 2 to pH 3, or a high concentration of a chaotrope, such as urea or thiocyanate ion), and TRICH is collected.

XVII. Identification of Molecules Which Interact with TRICH

Molecules which interact with TRICH may include transporter substrates, agonists or antagonists, modulatory proteins such as Gbg proteins (Reimann, supra) or proteins involved in TRICH localization or clustering such as MAGUKs (Craven, supra). TRICH, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled TRICH, washed, and any wells with labeled TRICH complex are assayed. Data obtained using different concentrations of TRICH are used to calculate values for the number, affinity, and association of TRICH with the candidate molecules.

Alternatively, proteins that interact with TRICH are isolated using the yeast 2-hybrid system as described in Fields, S. and C. Song (1989) Nature 340:245-246, or using commercially available kits based on the two-hybrid system, such as the MATCHMAKER system (Clontech). TRICH, or fragments thereof, are expressed as fusion proteins with the DNA binding domain of Gal4 or lexA, and potential interacting proteins are expressed as fusion proteins with an activation domain. Interactions between the TRICH fusion protein and the TRICH interacting proteins (fusion proteins with an activation domain) reconstitute a transactivation function that is observed by expression of a reporter gene. Methods for use of the yeast 2-hybrid system with ion channel proteins are discussed in Niethammer, M. and M. Sheng (1998, Meth. Enzymol. 293:104-122).

TRICH may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

Potential TRICH agonists or antagonists may be tested for activation or inhibition of TRICH ion channel activity using the assays described in section XIX.

XVIII. Demonstration of TRICH Activity

Ion channel activity of TRICH is demonstrated using an electrophysiological assay for ion conductance. TRICH can be expressed by transforming a mammalian cell line such as COS7, HeLa or CHO with a eukaryotic expression vector encoding TRICH. Eukaryotic expression vectors are commercially available, and the techniques to introduce them into cells are well known to those skilled in the art. A second plasmid which expresses any one of a number of marker genes, such as β-galactosidase, is co-transformed into the cells to allow rapid identification of those cells which have taken up and expressed the foreign DNA. The cells are incubated for 48-72 hours after transformation under conditions appropriate for the cell line to allow expression and accumulation of TRICH and β-galactosidase.

Transformed cells expressing β-galactosidase are stained blue when a suitable colorimetric substrate is added to the culture media under conditions that are well known in the art. Stained cells are tested for differences in membrane conductance by electrophysiological techniques that are well known in the art. Untransformed cells, and/or cells transformed with either vector sequences alone or β-galactosidase sequences alone, are used as controls and tested in parallel. Cells expressing TRICH will have higher anion or cation conductance relative to control cells. The contribution of TRICH to conductance can be confirmed by incubating the cells using antibodies specific for TRICH. The antibodies will bind to the extracellular side of TRICH, thereby blocking the pore in the ion channel, and the associated conductance.

Alternatively, ion channel activity of TRICH is measured as current flow across a TRICH-containing Xenopus laevis oocyte membrane using the two-electrode voltage-clamp technique (Ishi et al., supra; Jegla, T. and L. Salkoff (1997) J. Neurosci. 17:32-44). TRICH is subcloned into an appropriate Xenopus oocyte expression vector, such as pBF, and 0.5-5 ng of mRNA is injected into mature stage IV oocytes. Injected oocytes are incubated at 18° C. for 1-5 days. Inside-out macropatches are excised into an intracellular solution containing 116 mM K-gluconate, 4 mM KCl, and 10 mM Hepes (pH 7.2). The intracellular solution is supplemented with varying concentrations of the TRICH mediator, such as cAMP, cGMP, or Ca⁺² (in the form of CaCl₂), where appropriate. Electrode resistance is set at 2-5 MW and electrodes are filled with the intracellular solution lacking mediator. Experiments are performed at room temperature from a holding potential of 0 mV. Voltage ramps (2.5 s) from −100 to 100 mV are acquired at a sampling frequency of 500 Hz. Current measured is proportional to the activity of TRICH in the assay.

For example, the activity of TRICH-8 is measured as voltage-gated Cl— conductance, the activity of TRICH-14 is measured as a voltage-dependent anion channel, and the activity of TRICH-15 is measured as K⁺ conductance.

Transport activity of TRICH is assayed by measuring uptake of labeled substrates into Xenopus laevis oocytes. Oocytes at stages V and VI are injected with TRICH mRNA (10 ng per oocyte) and incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of TRICH. Oocytes are then transferred to standard uptake medium (100 mM NaCl, 2 mM KCl, 1 mM CaCl₂, 1 mM MgCl₂, 10 mM Hepes/Tris pH 7.5). Uptake of various substrates (e.g., amino acids, sugars, drugs, ions, and neurotransmitters) is initiated by adding labeled substrate (e.g. radiolabeled with ³H, fluorescently labeled with rhodamine, etc.) to the oocytes. After incubating for 30 minutes, uptake is terminated by washing the oocytes three times in Na⁺-free medium, measuring the incorporated label, and comparing with controls. TRICH activity is proportional to the level of internalized labeled substrate. Test substrates include aminophospholipids and other amphipathic molecules for TRICH-16.

ATPase activity associated with TRICH can be measured by hydrolysis of radiolabeled ATP-[g-³²P], separation of the hydrolysis products by chromatographic methods, and quantitation of the recovered ³²P using a scintillation counter. The reaction mixture contains ATP-[g-³²P] and varying amounts of TRICH in a suitable buffer incubated at 37° C. for a suitable period of time. The reaction is terminated by acid precipitation with trichloroacetic acid and then neutralized with base, and an aliquot of the reaction mixture is subjected to membrane or filter paper-based chromatography to separate the reaction products. The amount of ³²P liberated is counted in a scintillation counter. The amount of radioactivity recovered is proportional to the ATPase activity of TRICH in the assay.

TRICH fatty acid transport protein activity can be measured with very long chain acyl-CoA synthetase assay (Coe, N. R. et al. (1999) J. Biol. Chem. 274:36300-36304). Samples containing TRICH are assayed for palmitoyl-CoA and lignoceroyl-CoA synthetase activity by conversion of ³H-labeled palmitic acid or ¹⁴C-labeled lignoceric acid into their CoA derivatives. For solubilization of long chain and very long chain fatty acids, palmitic and lignoceric acids were dried under nitrogen and solubilized in 50 μl of -cyclodextrin (10 mg/ml) before use (Watkins, P. A. et al. (1998) J. Biol. Chem. 273, 18210-18219).

XIX. Identification of TRICH Agonists and Antagonists

TRICH is expressed in a eukaryotic cell line such as CHO (Chinese Hamster Ovary) or HEK (Human Embryonic Kidney) 293. Ion channel activity of the transformed cells is measured in the presence and absence of candidate agonists or antagonists. Ion channel activity is assayed using patch clamp methods well known in the art or as described in Example XVIII. Alternatively, ion channel activity is assayed using fluorescent techniques that measure ion flux across the cell membrane (Velicelebi, G. et al. (1999) Meth. Enzymol. 294:20-47; West, M. R. and C. R. Molloy (1996) Anal. Biochem. 241:51-58). These assays may be adapted for high-throughput screening using microplates. Changes in internal ion concentration are measured using fluorescent dyes such as the Ca²⁺ indicator Fluo-4 AM, sodium-sensitive dyes such as SBFI and sodium green, or the Cl⁻ indicator MQAE (all available from Molecular Probes) in combination with the FLIPR fluorimetric plate reading system (Molecular Devices). In a more generic version of this assay, changes in membrane potential caused by ionic flux across the plasma membrane are measured using oxonyl dyes such as DiBAC₄ (Molecular Probes). DiBAC₄ equilibrates between the extracellular solution and cellular sites according to the cellular membrane potential. The dye's fluorescence intensity is 20-fold greater when bound to hydrophobic intracellular sites, allowing detection of DiBAC₄ entry into the cell (Gonzalez, J. E. and P. A. Negulescu (1998) Curr. Opin. Biotechnol. 9:624-631). Candidate agonists or antagonists may be selected from known ion channel agonists or antagonists, peptide libraries, or combinatorial chemical libraries.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Incyte | | Incyte | | Incyte |
|---|---|---|---|---|
| Project | Polypeptide | Polypeptide | Polynucleotide | Polynucleotide |
| ID | SEQ ID NO: | ID | SEQ ID NO: | ID |
| 551243 | 1 | 551243CD1 | 18 | 551243CB1 |
| 7493587 | 2 | 7493587CD1 | 19 | 7493587CB1 |
| 4505840 | 3 | 4505840CD1 | 20 | 4505840CB1 |
| 7484873 | 4 | 7484873CD1 | 21 | 7484873CB1 |
| 3559054 | 5 | 3559054CD1 | 22 | 3559054CB1 |
| 7477526 | 6 | 7477526CD1 | 23 | 7477526CB1 |
| 7487253 | 7 | 7487253CD1 | 24 | 7487253CB1 |
| 2131556 | 8 | 2131556CD1 | 25 | 2131556CB1 |
| 3254315 | 9 | 3254315CD1 | 26 | 3254315CB1 |
| 7472707 | 10 | 7472707CD1 | 27 | 7472707CB1 |
| 7480432 | 11 | 7480432CD1 | 28 | 7480432CB1 |
| 7494181 | 12 | 7494181CD1 | 29 | 7494181CB1 |
| 3697053 | 13 | 3697053CD1 | 30 | 3697053CB1 |
| 7473203 | 14 | 7473203CD1 | 31 | 7473203CB1 |
| 4697002 | 15 | 4697002CD1 | 32 | 4697002CB1 |
| 5632139 | 16 | 5632139CD1 | 33 | 5632139CB1 |
| 7506184 | 17 | 7506184CD1 | 34 | 7506184CB1 |

TABLE 2

| Polypeptide | | GenBank ID NO: | | |
|---|---|---|---|---|
| SEQ | Incyte | or PROTEOME | Probability | |
| ID NO: | Polypeptide ID | ID NO: | Score | Annotation |
| 1 | 551243CD1 | g2605501 | 1.1E−64 | [Homo sapiens] OCTN1 (pH-dependent organic cation transporter) |
| | | | | Tamai, I. et al. (1997) FEBS Lett. 419: 107-111 |
| 2 | 7493587CD1 | g13878299 | 0.0 | [fl][Homo sapiens] aminophospholipid-transporting ATPase |
| 3 | 4505840CD1 | g3335567 | 1.9E−285 | [Mus musculus] fatty acid transport protein 3; FATP3 |
| | | | | Hirsch, D. et al. (1998) Proc. Natl. Acad. Sci. U.S.A. 95: 8625-8629 |
| 4 | 7484873CD1 | g19070539 | 0.0 | [fl][Homo sapiens] (AF348983) voltage-gated potassium channel Kv11.1 |
| 5 | 3559054CD1 | g17223622 | 0.0 | [fl][Homo sapiens] ATP-binding cassette A6 |
| 6 | 7477526CD1 | g18860924 | 0.0 | [fl][Homo sapiens] (AF350881) channel-kinase 2 |
| 7 | 7487253CD1 | g340199 | 2.0E−130 | [Homo sapiens] voltage-dependent anion channel |
| | | | | Blachly-Dyson, E. et al. (1993) Cloning and functional expression in yeast of two |
| | | | | human isoforms of the outer mitochondrial membrane channel, the voltage- |
| | | | | dependent anion channel. J. Biol. Chem. 268: 1835-1841 |
| 8 | 2131556CD1 | g4323622 | 1.0E−117 | [fl][Homo sapiens] intracellular chloride channel CLIC3 |
| 9 | 3254315CD1 | g7715417 | 0.0 | [Oryctolagus cuniculus] RING-finger binding protein |
| | | | | Mansharamani, M. et al. (2001) Cloning and Characterization of an Atypical Type |
| | | | | IV P-type ATPase that Binds to the RING Motif of RUSH Transcription Factors. |
| | | | | J. Biol. Chem. 276: 3641-3649 |
| 10 | 7472707CD1 | g16588684 | 0.0 | [fl][Homo sapiens] anion transporter/exchanger-8 |
| 11 | 7480432CD1 | g13278247 | 3.0E−37 | [fl][Mus musculus] nuclear transport factor 2 (placental protein 15) |
| 12 | 7494181CD1 | g14189735 | 0.0 | [fl][Homo sapiens] ATP-binding cassette transporter family A member 12 |
| 13 | 3697053CD1 | g8515881 | 2.6E−212 | [Rattus norvegicus] differentation-associated Na-dependent inorganic phosphate |
| | | | | cotransporter |
| 14 | 7473203CD1 | g5114261 | 2.1E−86 | [Homo sapiens] voltage-dependent anion channel isoform 2 |
| | | | | Decker, W. K. et al. (1999) Mamm. Genome 10: 1041-1042 |
| 15 | 4697002CD1 | g3880445 | 1.6E−30 | [Caenorhabditis elegans] contains similarity to Pfam domain: PF02214 (K+ |
| | | | | channel tetramerisation domain), Score = 79.5, E-value = 2.3e−20, N = 1 |
| | | | | The C. elegans Sequencing Consortium (1998) Science 282: 2012-2018 |
| 16 | 5632139CD1 | g6435130 | 0.0 | [Mus musculus] putative E1-E2 ATPase |
| | | | | Halleck, M. S., et al. (1999) Differential expression of putative transbilayer |
| | | | | amphipath |
| | | | | Transporters. Physiol. Genomics 1: 139-150 |
| 17 | 7506184CD1 | g3335567 | 1.2E−257 | [Mus musculus] fatty acid transport protein 3; FATP3 |
| | | | | (Hirsch, D. et al. (1998) Proc. Natl. Acad. Sci. U.S.A. 95: 8625-8629.) |
| | | 690690\| | 0.0 | [Homo sapiens] Protein with strong similarity to mouse Slc27a3, which is a fatty |
| | | MGC4365 | | acid transport protein that facilitates long chain fatty acid uptake across the plasma |
| | | | | membrane |
| | | 368728\| | 1.0E−258 | [Mus musculus][Transporter][Plasma membrane] Fatty acid transport protein, a |
| | | Slc27a3 | | plasma membrane protein facilitating long chain fatty acid uptake across the |
| | | | | plasma membrane, controls intracellular fatty acid concentration and plays roles in |
| | | | | energy homeostasis and diseases such as diabetes and obesity |
| | | | | (Hirsch, D. et al. (1998) Proc. Natl. Acad. Sci. USA 95: 8625-8629; Memon, R. A. |
| | | | | et al. (1999) Diabetes 48: 121-127.) |

TABLE 3

| | | Amino | | | | |
|---|---|---|---|---|---|---|
| SEQ | Incyte | Acid | Potential | Potential | | |
| ID | Polypeptide | Res- | Phosphorylation | Glycosylation | | Analytical Methods |
| NO: | ID | idues | Sites | Sites | Signature Sequences, Domains and Motifs | and Databases |
| 1 | 551243CD1 | 547 | S65 S108 S133 | N58 N63 N80 N106 | Signal peptide: M1-A33 | SPScan |
| | | | S223 S248 S259 | N286 | | |
| | | | S288 S294 S328 | | | |
| | | | S454 S500 S528 | | | |
| | | | S530 T368 T392 | | | |
| | | | T492 | | | |
| | | | | | Signal peptide: M13-L38 | HMMER |
| | | | | | Sugar (and other) transporter: V88-L496 | HMMER-PFAM |
| | | | | | Transmembrane domains: I15-G43, N106-D134, | TMAP |
| | | | | | K140-E163, L171-V191, G200-F220, I229-P249, | |
| | | | | | L298-T326, F363-L386, L403-T422, L435-K456, | |
| | | | | | K456-S478 | |
| | | | | | N-terminus is cytosolic | |
| | | | | | Sugar transport proteins signatures: I150-L216 | ProfileScan |
| 2 | 7493587CD1 | 1499 | S149 S185 S187 | N291 N413 N631 | Transmembrane domains: V92-A120, M306-I332, | TMAP |
| | | | S229 S300 S426 | N916 N1249 | V359-C387, L561-V583, L1090-C1110, | |
| | | | S445 S466 S479 | | T1116-L1136, V1198-E1222, T1227-C1252, | |
| | | | S489 S498 S501 | | T1264-F1292 | |
| | | | S507 S515 S525 | | N-terminus is non-cytosolic | |
| | | | S542 S634 S672 | | | |
| | | | S687 S697 S744 | | | |
| | | | S750 S780 S819 | | | |
| | | | S829 S835 S951 | | | |
| | | | S966 S971 S1049 | | | |
| | | | S1196 S1314 S1319 | | | |
| | | | S1334 S1340 S1371 | | | |
| | | | S1457 S1477 S1494 | | | |
| | | | S1495 T26 T66 | | | |
| | | | T268 T431 T443 | | | |
| | | | T584 T605 T614 | | | |
| | | | T861 T918 T1116 | | | |
| | | | T1311 T1343 | | | |
| | | | T1383 T1420 | | | |
| | | | | | E1-E2 ATPases phosphorylation site BL00154: | BLIMPS-BLOCKS |
| | | | | | G166-L183, I421-F439, K766-L776, D865-L905, | |
| | | | | | T1026-S1049 | |
| | | | | | E1-E2 ATPases phosphorylation site: R410-Q456 | ProfileScan |
| | | | | | P-type cation-transporting ATPase superfamily | BLIMPS-PRINTS |
| | | | | | signature PR00119: F425-F439, A881-D891, | |
| | | | | | I1029-I1048 | |
| | | | | | ATPase, hydrolase, transmembrane, probable calcium- | BLAST-PRODOM |
| | | | | | transporting: | |
| | | | | | PD004657: A1063-R1293 | |
| | | | | | PD006317: W160-I247 | |
| | | | | | PD149930: C1003-F1062 | |
| | | | | | ATPase, calcium transporting DM02405: | BLAST-DOMO |
| | | | | | P32660\|318-1225: E686-N1127, R150-E469, | |
| | | | | | F571-R590 | |
| | | | | | P39524\|236-1049: V100-G446, E944-N1127, | |
| | | | | | E684-E921, K546-V595, R460-R503 | |
| | | | | | S51243\|356-1267: Q685-F1126, E152-E469, | |
| | | | | | F571-R590 | |
| | | | | | Q09891\|206-1107: L703-N1127, E152-H494, | |
| | | | | | Y693-L931, F569-P602 | |
| | | | | | E1-E2 ATPases phosphorylation site: D427-T433 | MOTIFS |
| 3 | 4505840CD1 | 811 | S64 S96 S216 S220 | N118 N564 | Transmembrane Domains: P122-L150, A296-A323, | TMAP |
| | | | S222 S228 S254 | | P456-F483 | |
| | | | S272 S406 S516 | | N-terminus is non-cytosolic | |
| | | | S609 T593 T675 | | | |
| | | | T761 T778 T798 | | | |
| | | | | | Putative AMP-binding domain signature | PROFILESCAN |
| | | | | | amp_binding.prf: D394-G438 | |
| | | | | | Putative AMP-binding domain BL00455: F415-H430 | BLIMPS_BLOCKS |
| | | | | | AMP-binding signature PR00154: T408-T419, | BLIMPS_PRINTS |
| | | | | | T420-I428 | |
| | | | | | PUTATIVE AMP-BINDING DOMAIN | BLAST_DOMO |
| | | | | | DM00073\|A55093\|83-604: S293-M769 | |
| | | | | | DM00073\|P31552\|22-521: G295-K580, I598-L756, | |
| | | | | | R218-F235 DM00073\|P39846\|1507-1995: | |
| | | | | | L315-K766, Q205-R237 DM00073\|P41636\|32-530: | |
| | | | | | G286-A694 | |
| 4 | 7484873CD1 | 545 | S5 S10 S12 S137 | N17 N440 | Putative AMP-binding domain signature Y413-K424 | MOTIFS |
| | | | S211 S323 T19 T83 | | | |
| | | | T130 T195 T201 | | | |
| | | | T281 T403 T510 | | | |
| | | | T540 Y187 | | | |
| | | | | | K+ channel tetramerisation domain: S97-F203 | HMMER_PFAM |
| | | | | | Ion transport protein: I301-L492 | HMMER_PFAM |
| | | | | | Transmembrane Domains: V157-G175, S255-T281, | TMAP |
| | | | | | E303-S323, N336-T356, Q406-Y430, L469-F496 | |
| | | | | | Potassium channel signature PR00169: E148-S167, | BLIMPS_PRINTS |
| | | | | | P253-T281, H304-L327, F330-L350, L381-C407, | |
| | | | | | Q410-E433, F441-M463, G470-F496 | |
| | | | | | CHANNEL IONIC PROTEIN POTASSIUM | BLAST_PRODOM |
| | | | | | SUBUNIT VOLTAGEGATED | |
| | | | | | TRANSMEMBRANE CALCIUM TRANSPORT | |
| | | | | | ION PD000141: F330-K502 | |
| | | | | | do CHANNEL; POTASSIUM; CDRK; FORM; | BLAST_DOMO |
| | | | | | DM00436\|JH0595\|144-307: K215-L381 | |
| | | | | | DM00436\|P15387\|136-299: R206-L381 | |
| | | | | | DM00436\|P17970\|386-549: I216-L381 | |
| | | | | | DM00490\|P17970\|268-384: A94-R200 | |
| 5 | 3559054CD1 | 1583 | S30 S50 S134 S249 | N71 N84 N91 N109 | Signal Peptide: M26-I44 | HMMER |
| | | | S353 S491 S632 | N130 N241 N436 | | |
| | | | S721 S752 S775 | N544 N576 N877 | | |
| | | | S785 S881 S889 | N906 N956 N1271 | | |
| | | | S920 S1001 S1093 | | | |
| | | | S1159 S1235 S1261 | | | |
| | | | S1295 S1454 T111 | | | |
| | | | T206 T558 T572 | | | |
| | | | T603 T715 T732 | | | |
| | | | T740 T818 T934 | | | |
| | | | T1138 T1223 | | | |
| | | | T1306 T1336 | | | |
| | | | T1384 T1407 | | | |
| | | | T1428 T1511 | | | |
| | | | T1571 Y913 | | | |
| | | | | | ABC transporter: G1279-G1455, G507-G649 | HMMER_PFAM |
| | | | | | Transmembrane domain: R25-N53 E221-K247 | TMAP |
| | | | | | A262-V282 I292-V312 L322-L342 E356-N382 | |
| | | | | | D392-I420 L814-Y842 H972-G1000 Q1027-Y1047 | |
| | | | | | V1061-M1081 F1098-V1126 C1166-M1192 | |
| | | | | | N-terminus is non-cytosolic | |
| 5 | | | | | ABC TRANSPORTERS FAMILY | BLAST_DOMO |
| | | | | | DM00008\|P41233\|839-1045: K1266-M1452, | |
| | | | | | I478-P607, E594-N648 | |
| | | | | | DM00008\|P41233\|1851-2058: K1262-S1454, | |
| | | | | | I478-V591, I595-N648 | |
| | | | | | DM00008\|P34358\|611-816: A1268-M1452, | |
| | | | | | I478-D599, E592-N648 | |
| | | | | | DM00008\|P23703\|41-246: E1251-G1455, L500-I595, | |
| | | | | | E592-G649 | |
| | | | | | ATP/GTP-binding site motif A (P-loop) G514-S521 | MOTIFS |
| | | | | | G1280-S1293 | |
| 6 | 7477526CD1 | 2004 | S982 S1121 S1150 | N130 N338 N387 | Ion transport protein: Y891-F1051 | HMMER_PFAM |
| | | | S1159 S7 S1208 | N510 N538 N576 | | |
| | | | S1226 S725 S1288 | N649 N683 N686 | | |
| | | | S27 S1350 S1381 | N769 N893 N1148 | | |
| | | | S72 S1440 S1449 | N1484 N1504 | | |
| | | | S284 S1460 S180 | N1540 N1912 | | |
| | | | S1466 S1467 S772 | N1973 | | |
| | | | S1480 S340 S1485 | | | |
| | | | S1523 S568 | | | |
| | | | S1565 S389 S1585 | | Transmembrane domains: L484-I503, R718-S746, | TMAP |
| | | | S1598 S1600 S572 | | P808-L832, Q853-E871, N893-R910, H917-V941, | |
| | | | S1646 S1681 S778 | | A957-I980, F1026-V1054 | |
| | | | S1684 S746 S1693 | | | |
| | | | S1741 S1753 S1772 | | | |
| | | | S851 S1984 S1997 | | | |
| | | | T69 T362 T491 | | PROTEIN MELASTATIN CHROMOSOME | BLAST_PRODOM |
| | | | T512 T617 T810 | | TRANSMEMBRANE: PD018035: Y93-P419 | |
| | | | T881 T1076 T1203 | | PD039592: R569-D758 PD151509: D994-K1205, | |
| | | | T311 T1212 T288 | | R934-G1024 PD022180: H416-Y524 | |
| | | | T1227 T96 T1357 | | | |
| | | | T1571 T100 T1617 | | | |
| | | | T1770 T1837 | | | |
| | | | T1881 T301 T1897 | | | |
| | | | T1993 | | | |
| 7 | 7487253CD1 | 281 | S13 S137 T6 T33 | N124 N239 | Eukaryotic porin: A2-V281 | HMMER_PFAM |
| | | | T51 T70 T72 T86 | | | |
| | | | T107 T159 T217 | | | |
| | | | T250 | | | |
| | | | | | Eukaryotic mitochondrial porin BL00558: G56-L69, | BLIMPS_BLOCKS |
| | | | | | T80-S104 | |
| | | | | | Eukaryotic mitochondrial porin signature: L39-S104 | PROFILESCAN |
| | | | | | Eukaryotic porin signature PR00185: P5-K20, | BLIMPS_PRINTS |
| | | | | | G68-T83, E147-E158, Y247-Y264 | |
| | | | | | PORIN CHANNEL VOLTAGE-DEPENDENT | BLAST_PRODOM |
| | | | | | OUTER MEMBRANE PROTEIN | |
| | | | | | MITOCHONDRION ANION-SELECTIVE | |
| | | | | | MITOCHONDRIAL PD003211: P4-Q280 | |
| | | | | | EUKARYOTIC MITOCHONDRIAL PORIN | BLAST_DOMO |
| | | | | | DM01893\|P45879\|1-282: A2-Q280 | |
| | | | | | DM01893\|A38102\|14-296: V3-Q280 | |
| | | | | | DM01893\|P45880\|28-346: V3-L273 | |
| | | | | | DM01893\|A45972\|28-347: V3-L273 | |
| 8 | 2131556CD1 | 236 | S159 T42 T46 | | signal_cleavage: M1-T42 | SPSCAN |
| | | | T170 | | | |
| | | | | | Transmembrane domain: Q26-S49 | TMAP |
| | | | | | N-terminus is cytosolic. | |
| | | | | | PROTEIN CHANNEL IONIC ION TRANSPORT | BLAST_PRODOM |
| | | | | | VOLTAGEGATED P64 CHLORIDE | |
| | | | | | INTRACELLULAR CHLORINE PD017366: E3-I225 | |
| 9 | 3254315CD1 | 1177 | S46 S115 S163 | N331 N390 N449 | E1-E2 ATPase: V126-D164 | HMMER_PFAM |
| | | | S276 S280 S332 | N461 N477 N786 | | |
| | | | S470 S520 S527 | N998 | | |
| | | | S528 S574 S580 | | | |
| | | | S737 S929 S957 | | | |
| | | | S1154 S1170 T262 | | | |
| | | | T406 T411 T413 | | | |
| | | | T473 T636 T678 | | | |
| | | | T753 T906 T1014 | | | |
| | | | T1100 T1102 Y322 | | | |
| | | | | | haloacid dehalogenase-like hydrolase: V401-E842 | HMMER_PFAM |
| | | | | | Transmembrane domains: E61-P84, S86-K104, | TMAP |
| | | | | | V282-L307, F338-F366, R856-Y884, V911-L930, | |
| | | | | | T960-I983, F1001-T1021, W1033-F1053, | |
| | | | | | Q1065-F1085 | |
| | | | | | N-terminus is cytosolic. | |
| | | | | | E1-E2 ATPases phosphorylation sik BL00154: | BLIMPS_BLOCKS |
| | | | | | G143-L160, V401-F419, K595-C605, D682-H722, | |
| | | | | | T816-M839 | |
| 9 | | | | | E1-E2 ATPases phosphorylation site: A387-L436 | PROFILESCAN |
| | | | | | P-type cation-transporting atpase superfamily | BLIMPS_PRINTS |
| | | | | | signature PR00119: F405-F419, A698-D708, | |
| | | | | | V819-I838 | |
| | | | | | H+-transporting ATPase (proton pump) signature | BLIMPS_PRINTS |
| | | | | | PR00120: T613-A631, V819-G835 | |
| | | | | | Sodium/potassium-transporting ATPase signature | BLIMPS_PRINTS |
| | | | | | PR00121: V88-E108, L398-F419, L592-I610 | |
| | | | | | ATPASE HYDROLASE TRANSMEMBRANE | BLAST_PRODOM |
| | | | | | PHOSPHORYLATION ATP-BINDING CALCIUM | |
| | | | | | TRANSPORT PD004657: A853-K1104 PD149930: | |
| | | | | | C792-Y852 PD006317: G130-M224 | |
| | | | | | ATPASE; CALCIUM; TRANSPORTING | BLAST_DOMO |
| | | | | | DM02405\|P39524\|236-1049: F488-N917, T83-D506, | |
| | | | | | S169-L201 | |
| | | | | | DM02405\|Q09891\|206-1107: K134-E777, | |
| | | | | | H760-N917 | |
| | | | | | DM02405\|S51243\|356-1267: K138-I601, E484-H776, | |
| | | | | | S737-Y916 | |
| | | | | | DM02405\|P32660\|318-1225: K138-I601, K138-I601, | |
| | | | | | E484-E777, E754-N917, T310-D341 | |
| | | | | | E1-E2 ATPases phosphorylation site: D407-T413 | MOTIFS |
| 10 | 7472707CD1 | 970 | S13 S16 S20 S44 | N52 N192 N277 | STAS (Sulphate Transporter and Anti-Sigma factor | HMMER_PFAM |
| | | | S389 S521 S540 | N384 N595 N651 | antagonist) domain: Y544-A791 | |
| | | | S645 S669 S689 | N687 N688 | | |
| | | | S787 S797 S807 | | | |
| | | | S820 S839 S858 | | | |
| | | | S866 S966 T55 | | | |
| | | | T334 T518 T625 | | | |
| | | | T655 T818 T822 | | | |
| | | | T896 T898 T906 | | | |
| | | | | | Sulfate transporter family: M212-S521 | HMMER_PFAM |
| | | | | | Transmembrane domains: R71-W91, L104-L124, | TMAP |
| | | | | | Y137-L157, V198-E226, M241-S261, C270-A290, | |
| | | | | | C296-K324, P355-H383, L393-T418, F430-F450, | |
| | | | | | L458-S478, M491-V519, V741-F769 | |
| | | | | | N-terminus is cytosolic. | |
| | | | | | Sulfate transporters protein BL01130: I100-L153, | BLIMPS_BLOCKS |
| | | | | | T200-I251 | |
| | | | | | SULFATE TRANSPORTER TRANSMEMBRANE | BLAST_PRODOM |
| | | | | | GLYCOPROTEIN AFFINITY SULPHATE HIGH | |
| | | | | | PERMEASE PD001121: I76-V198 | |
| | | | | | SULFATE TRANSPORTER TRANSMEMBRANE | BLAST_PRODOM |
| | | | | | AFFINITY GLYCOPROTEIN HIGH DISEASE | |
| | | | | | PD001755: R523-D579, V721-K799 | |
| | | | | | SULFATE TRANSPORTER TRANSMEMBRANE | BLAST_PRODOM |
| | | | | | PERMEASE INTERGENIC REGION AFFINITY | |
| | | | | | GLYCOPROTEIN PD001255: S352-S512, | |
| | | | | | M212-A322 | |
| | | | | | SULFATE TRANSPORTERS | BLAST_DOMO |
| | | | | | DM01229\|P40879\|5-462: Y23-R481 | |
| | | | | | DM01229\|P45380\|10-468: C62-R481 | |
| | | | | | DM01229\|P50443\|49-505: C62-W480 | |
| | | | | | DM01229\|P53393\|11-447: R68-R481 | |
| 11 | 7480432CD1 | 179 | S18 S57 | N25 | Nuclear transport factor 2 (NTF2) domain: T10-P122 | HMMER_PFAM |
| | | | | | NUCLEAR TRANSPORT FACTOR NTF2 | BLAST_PRODOM |
| | | | | | PLACENTAL 3D-STRUCTURE PD012808: | |
| | | | | | N25-L124 | |
| 12 | 7494181CD1 | 1662 | S55 S64 S400 S511 | N237 N591 N730 | ABC transporter: G438-G620, G1350-G1532 | HMMER_PFAM |
| | | | S537 S555 S580 | N771 N836 N886 | | |
| | | | S583 S611 S648 | N902 N943 N988 | | |
| | | | S691 S735 S760 | N1019 N1245 | | |
| | | | S840 S915 S1021 | N1275 N1290 | | |
| | | | S1050 S1231 S1269 | N1342 N1385 | | |
| | | | S1466 S1485 S1491 | N1609 N1614 | | |
| | | | S1515 S1586 S1655 | | | |
| | | | T51 T61 T104 | | Transmembrane domains: G4-L20, F128-L155, | TMAP |
| | | | T482 T501 T518 | | N173-K201, N210-Y230, A242-T262, S268-R292, | |
| | | | T529 T574 T593 | | S316-R339, T454-Y472, T807-R835, | |
| | | | T644 T722 T723 | | T1047-V1075, T1100-L1125, L1134-L1154, | |
| | | | T747 T761 T773 | | G1163-S1183, L1207-I1224, N1251-I1279 | |
| | | | T781 T807 T833 | | N-terminus is cytosolic. | |
| | | | T903 T956 T1206 | | | |
| | | | T1297 T1414 | | | |
| | | | T1461 T1552 | | | |
| | | | T1608 T1623 | | | |
| | | | T1624 Y37 Y536 | | | |
| | | | Y1001 | | | |
| | | | | | ABC transporters family BL00211: L443-T454, | BLIMPS_BLOCKS |
| | | | | | L546-D577 | |
| | | | | | ABC transporters family signature: V526-D577 | PROFILESCAN |
| | | | | | ATP-BINDING TRANSPORTER CASSETTE ABC | BLAST_PRODOM |
| | | | | | GLYCOPROTEIN TRANSMEMBRANE RIM | |
| | | | | | ABCR ABCC PD006285: N238-Y422 PD005939: | |
| | | | | | I1048-G1220 PD006867: L91-N237 PD007075: | |
| | | | | | H640-I846 | |
| | | | | | ABC TRANSPORTERS FAMILY | BLAST_DOMO |
| | | | | | DM00008\|P41233\|839-1045: V413-L617, | |
| | | | | | V1321-M1529 | |
| | | | | | DM00008\|P41233\|1851-2058: L1320-N1531, | |
| | | | | | V418-L617 | |
| | | | | | DM00008\|P34358\|611-816: V413-D612, | |
| | | | | | A1339-M1529 | |
| | | | | | ABC transporters family signature: L546-L560 | MOTIFS |
| | | | | | ATP/GTP-binding site motif A (P-loop): G445-T452, | MOTIFS |
| | | | | | G1357-T1364 | |
| 13 | 3697053CD1 | 588 | S151 S279 S289 | N105 N106 N201 | Transmembrane Domains: P73-V101, T128-G148, | TMAP |
| | | | S540 S546 T8 T57 | N216 N513 N583 | A156-Y182, A233-Y261, P305-T328, F340-I368, | |
| | | | T284 T475 T568 | | A400-F428, M470-G498 | |
| | | | | | N-terminus is cytosolic | |
| | | | | | BRAIN SPECIFIC NA+DEPENDENT INORGANIC | BLAST_PRODOM |
| | | | | | PHOSPHATE COTRANSPORTER PD063887: | |
| | | | | | K18-Q118 | |
| | | | | | PHOSPHATE; TRANSPORT; SODIUM; RENAL; | BLAST_DOMO |
| | | | | | DM01845\|I59302\|222-505: M234-E517 | |
| | | | | | DM01845\|P34644\|215-507: A222-E516 | |
| | | | | | DM01845\|Q03567\|156-455: H223-A509 | |
| | | | | | DM02667\|I59302\|45-180: P62-L194 | |
| | | | | | Immunoglobulins and major histocompatibility | MOTIFS |
| | | | | | complex proteins signature Y269-H275 | |
| 14 | 7473203CD1 | 257 | S82 S88 S130 S223 | N80 N139 N185 | signal_cleavage: M1-A24 | SPSCAN |
| | | | T62 | N207 N208 | | |
| | | | | | Eukaryotic porin: W13-T257 | HMMER_PFAM |
| | | | | | Transmembrane Domains: D92-W120 | TMAP |
| | | | | | N-terminus is cytosolic | |
| | | | | | Eukaryotic porin signature PR00185: S16-K31, | BLIMPS_PRINTS |
| | | | | | E118-D129, L221-D238 | |
| | | | | | PORIN CHANNEL VOLTAGEDEPENDENT | BLAST_PRODOM |
| | | | | | OUTER MEMBRANE PROTEIN | |
| | | | | | MITOCHONDRION ANIONSELECTIVE | |
| | | | | | MITOCHONDRIAL VDAC PD003211: G79-E256, | |
| | | | | | Y18-K74 | |
| | | | | | EUKARYOTIC MITOCHONDRIAL PORIN | BLAST_DOMO |
| | | | | | DM01893\|A38102\|14-296: T78-E256, T78-V214, | |
| | | | | | I14-K74 | |
| | | | | | DM01893\|A45972\|28-347: T78-G250, T78-V214, | |
| | | | | | I14-K74 DM01893\|P45880\|28-346: T78-G250, | |
| | | | | | T78-V214, I14-K74 DM01893\|P45879\|1-282: | |
| | | | | | T78-E256, T78-V214, Y18-K74 | |
| 15 | 4697002CD1 | 473 | S64 S78 S147 S155 | N168 N253 N294 | signal_cleavage: M1-P39 | SPSCAN |
| | | | S163 S313 S328 | N424 | | |
| | | | S351 S358 S362 | | | |
| | | | S393 S410 S426 | | | |
| | | | S435 T219 T251 | | | |
| | | | T266 T274 T342 | | | |
| | | | T368 Y136 | | | |
| | | | | | K+ channel tetramerisation domain: E44-T151 | HMMER_PFAM |
| | | | | | Potassium channel signature PR00169: R96-D115, | BLIMPS_PRINTS |
| | | | | | F229-S255 | |
| | | | | | CHANNEL; POTASSIUM; CDRK; SHAW; | BLAST_DOMO |
| | | | | | DM00490\|S13919\|27-144: E44-P121 | |
| 16 | 5632139CD1 | 1095 | S82 S204 S371 | N310 N464 N529 | E1-E2 ATPase domain: L277-T305, G171-V199 | HMMER_PFAM |
| | | | S486 S535 S536 | | | |
| | | | S585 S596 S661 | | | |
| | | | S666 S745 S872 | | | |
| | | | S957 S1086 T168 | | | |
| | | | T237 T265 T301 | | | |
| | | | T402 T415 T422 | | | |
| | | | T442 T686 T744 | | | |
| | | | T800 T813 T1009 | | | |
| | | | Y648 | | | |
| | | | | | Transmembrane domain: I94-L122, L127-F153, | TMAP |
| | | | | | E280-V308, N326-L346, G351-S371, G880-V900, | |
| | | | | | P907-F927, F958-G978, E986-V1006, | |
| | | | | | W1015-L1035, I1041-L1061 | |
| | | | | | N-terminus is non-cytosolic | |
| | | | | | E1-E2 ATPases phosphorylation site signature | BLIMPS_BLOCKS |
| | | | | | BL00154: G183-F200, L410-F428, D690-L730, | |
| | | | | | T817-E840 | |
| | | | | | E1-E2 ATPases phosphorylation site: T396-D444 | PROFILESCAN |
| | | | | | P-type cation-transporting atpase superfamily | BLIMPS_PRINTS |
| | | | | | signature PR00119: L414-F428, A706-D716, | |
| | | | | | I820-I839 | |
| | | | | | ATPASE HYDROLASE TRANSMEMBRANE | BLAST_PRODOM |
| | | | | | PHOSPHORYLATION ATP-BINDING PROTEIN | |
| | | | | | PROBABLE CALCIUM-TRANSPORTING | |
| | | | | | CALCIUM TRANSPORT PD004657: S854-L1093 | |
| | | | | | PD149930: C794-F853 PD006317: K176-D270 | |
| | | | | | PD034942: L122-T168 | |
| | | | | | ATPASE; CALCIUM; TRANSPORTING; | BLAST_DOMO |
| | | | | | DM02405\|P40527\|208-977: F525-A918, S116-E507 | |
| | | | | | E1-E2 ATPases phosphorylation site: D416-T422 | MOTIFS |
| 17 | 7506184CD1 | 758 | S64 S96 S216 S220 | N118 N564 | AMP-binding enzyme: L292-Y565, G611-V650, | HMMER_PFAM |
| | | | S222 S228 S254 | | S220-G240 | |
| | | | S272 S406 S516 | | | |
| | | | T593 T622 T708 | | | |
| | | | T725 T745 | | | |
| | | | | | Putative AMP-binding domain proteins BL00455: | BLIMPS_BLOCKS |
| | | | | | F415-H430 | |
| | | | | | Putative AMP-binding domain signature: D394-G438 | PROFILESCAN |
| | | | | | AMP-BINDING SIGNATURE PR00154: T408-T419, | BLIMPS_PRINTS |
| | | | | | T420-I428 | |
| | | | | | PROTEIN FATTY ACID TRANSMEMBRANE | BLAST_PRODOM |
| | | | | | VERY LONG CHAIN ACYL COA SYNTHETASE | |
| | | | | | TRANSPORT VERY LONG CHAIN FATTY ACID | |
| | | | | | COA PD007209: Y651-I758 | |
| | | | | | PUTATIVE AMP-BINDING DOMAIN | BLAST_DOMO |
| | | | | | DM00073\|A55093\|83-604: S293-G611, G611-M716 | |
| | | | | | DM00073\|P31552\|22-521: G295-K580, F612-L703, | |
| | | | | | R218-F235 | |
| | | | | | Putative AMP-binding domain signature: Y413-K424 | MOTIFS |

TABLE 4

| Polynucleotide | |
|---|---|
| SEQ ID NO:/ | |
| Incyte ID/Sequence | |
| Length | Sequence Fragments |
| 18/551243CB1/ | 1-270, 38-270, 42-270, 47-270, 47-659, 52-270, 56-332, 60-148, 61-360, 73-268, 73-270, 97-270, 97-360, 97-788, |
| 1929 | 117-664, 145-344, 145-360, 147-270, 147-360, 151-270, 244-270, 359-495, 588-1179, 642-948, 657-788, 820-1414, |
| | 823-985, 1004-1231, 1063-1316, 1063-1356, 1074-1687, 1109-1231, 1249-1888, 1397-1929, 1425-1704, 1425-1846, |
| | 1425-1894, 1425-1929, 1427-1712, 1530-1929, 1580-1929, 1612-1929, 1732-1929, 1797-1929, 1825-1929, |
| | 1831-1929, 1832-1929, 1868-1929 |
| 19/7493587CB1/ | 1-452, 1-585, 149-802, 303-362, 304-362, 314-361, 381-933, 531-772, 598-803, 889-995, 936-1471, 993-1511, 994-1225, |
| 5302 | 1259-1925, 1269-1511, 1376-1511, 1730-2492, 1929-2398, 2216-2481, 2263-2922, 2299-2492, 2363-2980, |
| | 2546-2793, 2567-3131, 2587-2883, 2587-3133, 2634-2902, 2634-2904, 2998-3264, 3132-3572, 3175-3388, 3314-3593, |
| | 3321-3539, 3394-3659, 3405-3820, 3454-4130, 3532-3808, 3532-3950, 3532-3972, 3532-4019, 3532-4030, |
| | 3532-4036, 3532-4039, 3532-4052, 3532-4087, 3532-4133, 3532-4268, 3533-3717, 3533-3748, 3548-4071, 3582-4058, |
| | 3590-4222, 3617-4124, 3651-3905, 3710-3850, 3721-4004, 3745-4076, 3748-3826, 3781-4048, 3790-4469, |
| | 3792-3862, 3840-4454, 3853-4368, 3870-4346, 3878-4179, 3917-4014, 3984-4249, 4036-4291, 4047-4661, 4126-4687, |
| | 4178-4426, 4178-4855, 4186-4659, 4258-4549, 4307-4488, 4312-4580, 4386-4622, 4386-4778, 4419-4671, |
| | 4457-4697, 4489-4652, 4502-4697, 4528-4777, 4539-4696, 4539-4867, 4549-4885, 4606-5266, 4707-5224, 4739-5287, |
| | 4741-5265, 4785-5302, 4817-5299, 4876-5170, 4923-4953, 5071-5289 |
| 20/4505840CB1/ | 1-437, 280-606, 343-902, 348-581, 348-627, 359-454, 359-605, 359-624, 393-941, 405-965, 632-1100, 736-1282, |
| 2994 | 832-1225, 998-1307, 1038-1252, 1038-1509, 1076-1348, 1085-1470, 1099-1726, 1108-1350, 1113-1314, 1143-1655, |
| | 1207-1783, 1227-1589, 1272-1549, 1277-1837, 1289-1542, 1289-1825, 1291-1941, 1301-1523, 1301-1785, |
| | 1310-1836, 1335-1625, 1349-1922, 1353-1643, 1361-1815, 1372-1618, 1390-1683, 1397-1687, 1422-1651, 1448-2004, |
| | 1453-2015, 1510-1852, 1534-1780, 1571-1856, 1594-1789, 1632-2283, 1677-2255, 1705-2355, 1710-2215, |
| | 1712-1992, 1753-2312, 1757-1992, 1770-2004, 1789-2288, 1795-2000, 1795-2366, 1800-2015, 1946-2013, 1970-2241, |
| | 1977-2625, 1997-2268, 2010-2172, 2015-2319, 2055-2601, 2062-2622, 2065-2317, 2082-2350, 2094-2621, |
| | 2117-2365, 2162-2384, 2171-2413, 2200-2463, 2206-2449, 2236-2560, 2285-2488, 2301-2518, 2304-2536, 2304-2621, |
| | 2304-2634, 2305-2626, 2355-2599, 2410-2649, 2420-2676, 2420-2994 |
| 21/7484873CB1/ | 1-444, 1-471, 1-596, 1-597, 1-644, 18-854, 142-1692, 342-963, 346-963, 355-963, 392-963, 1498-1692, 1501-1538, |
| 2094 | 1501-1543, 1661-2086, 1661-2092, 1661-2093, 1661-2094, 1665-2094, 1719-2088, 1851-2088, 1885-2088 |
| 22/3559054CB1/ | 1-274, 6-483, 10-274, 26-299, 42-317, 42-445, 42-524, 42-551, 42-595, 42-603, 42-607, 42-622, 42-623, 42-638, 42-643, |
| 5846 | 42-645, 42-662, 42-674, 42-675, 42-680, 42-681, 43-317, 43-464, 48-358, 49-330, 58-319, 83-327, 85-544, 85-600, |
| | 94-623, 95-364, 222-4973, 318-522, 318-681, 523-681, 523-785, 682-1012, 786-1012, 786-1154, 1013-1154, |
| | 1013-1340, 1155-1340, 1155-1488, 1341-1488, 1489-1657, 1489-1716, 1658-1827, 1717-2003, 1828-2003, 1828-2142, |
| | 2004-2142, 2143-2373, 2234-2373, 2234-2508, 2374-2508, 2374-2692, 2541-2692, 2551-3151, 2557-3025, |
| | 2602-3299, 2655-3442, 2693-2859, 2693-2993, 2724-3441, 2860-3131, 2889-3442, 2913-3395, 2913-3406, 2913-3441, |
| | 2913-3442, 2919-3441, 2994-3131, 3132-3415, 3210-3432, 3210-3625, 3240-3415, 3240-3527, 3280-3555, |
| | 3421-3647, 3449-3783, 3449-3832, 3449-3851, 3449-3972, 3449-4026, 3482-3671, 3525-4168, 3528-3725, 3530-4013, |
| | 3562-3946, 3602-3849, 3634-3928, 3657-3817, 3669-4341, 3726-3938, 3765-4216, 3767-4242, 3768-4106, |
| | 3769-4229, 3777-4103, 3783-4033, 3818-4056, 3820-4454, 3835-4072, 3850-4284, 3905-4296, |
| | 3908-4176, 3927-4277, 3928-4446, 3938-4206, 3939-4148, 3943-4190, 3950-4211, 3953-4454, 3982-4246, 4008-4218, |
| | 4016-4258, 4016-4278, 4021-4491, 4062-4441, 4092-4353, 4092-4790, 4118-4401, 4120-4441, 4120-4570, |
| | 4145-4570, 4149-4303, 4149-4379, 4159-4746, 4197-4931, 4239-4446, 4251-4443, 4257-4591, 4294-4924, 4300-4818, |
| | 4304-4474, 4310-5064, 4319-4939, 4344-4595, 4344-4993, 4371-4914, 4375-5021, 4380-4594, 4389-5001, |
| | 4392-4916, 4396-4907, 4398-5027, 4421-4988, 4439-4954, 4461-5018, 4461-5026, 4475-4735, 4482-4707, 4493-4825, |
| | 4518-4788, 4518-4917, 4524-5205, 4556-5026, 4571-4881, 4590-4786, 4590-4803, 4592-5077, 4595-4735, |
| | 4595-4815, 4596-4860, 4597-4875, 4607-5240, 4633-4928, 4633-4995, 4643-5026, 4655-4922, 4660-5211, 4706-4852, |
| | 4723-5026, 4736-4871, 4738-5040, 4738-5157, 4782-5121, 4786-5233, 4815-5033, 4816-4973, 4836-4993, |
| | 4836-5121, 4838-4992, 4865-5225, 4905-5241, 4909-5147, 4909-5236, 4910-5255, 4947-5213, 4990-5258, 5033-5505, |
| | 5117-5303, 5212-5464, 5212-5846 |
| 23/7477526CB1/ | 1-407, 1-581, 1-585, 1-615, 1-616, 1-626, 322-963, 322-964, 324-963, 324-964, 328-963, 335-960, 500-737, 798-1563, |
| 6813 | 803-1096, 925-1389, 953-1552, 953-1740, 954-1740, 975-1389, 1022-1738, 1025-1582, 1025-1690, 1025-1700, |
| | 1025-1740, 1026-1740, 1028-1740, 1050-1752, 1060-1645, 1060-1752, 1060-1781, 1060-1824, 1060-1835, |
| | 1060-1862, 1060-1877, 1062-1748, 1069-1832, 1069-1865, 1074-1831, 1390-1740, 1521-2108, 1521-2211, 1521-2214, |
| | 1521-2217, 1521-2227, 1521-2240, 1521-2252, 1524-2143, 1699-2646, 1831-2480, 1831-2487, 1871-2444, |
| | 1871-2453, 1871-2469, 1871-2480, 1871-2482, 2199-2806, 2199-2813, 2199-2818, 2199-2829, 2199-2831, 2209-2831, |
| | 2210-2831, 2220-2829, 2220-2832, 2220-2924, 2221-2815, 2221-2831, 2221-2924, 2223-2924, 2227-2924, |
| | 2228-2566, 2240-2924, 2245-2924, 2253-2924, 2265-2910, 2269-2817, 2269-2906, 2269-2923, 2269-2924, 2271-2817, |
| | 2273-2924, 2281-2924, 2466-2913, 2793-3083, 2793-3349, 2797-3349, 2809-3349, 2860-2922, 2862-3349, |
| | 2865-3349, 3174-6813, 3285-3456, 3298-3791, 3364-3791, 3715-3875, 3814-4500, 3919-4537, 3919-4592, 3919-4601, |
| | 3919-4614, 3919-4621, 3919-4622, 3919-4641, 3919-4653, 3919-4718, 4031-6404, 4204-4441, 4353-5161, |
| | 4471-4920, 4480-4920, 4495-4920, 4506-4920, 4508-4920, 4514-4920, 4528-4920, 4545-4920, 4555-4920, 4610-4920, |
| | 4627-4920, 5559-5823, 5559-6068, 5755-5991, 5755-6014, 6030-6250, 6239-6769, 6261-6548, 6261-6773, |
| | 6273-6551, 6568-6813 |
| 24/7487253CB1/ | 1-416, 11-456, 19-289, 38-312, 38-470, 84-803, 100-353, 100-460, 102-331, 106-405, 106-534, 106-951, 122-391, |
| 951 | 127-388, 127-426, 136-386, 541-701, 705-951 |
| 25/2131556CB1/ | 1-838, 115-325, 115-358, 115-842, 116-245, 116-275, 116-340, 116-349, 116-474, 116-731, 117-400, 118-333, 118-364, |
| 925 | 118-365, 118-382, 119-368, 119-409, 119-718, 121-360, 122-360, 122-406, 124-261, 124-275, 125-251, 125-307, |
| | 125-381, 125-418, 125-446, 125-461, 125-524, 125-541, 125-552, 125-553, 125-614, 125-627, 125-650, 125-659, |
| | 125-661, 125-676, 125-684, 125-687, 125-704, 125-706, 125-707, 125-723, 125-748, 125-752, 125-754, 125-761, |
| | 125-765, 125-784, 125-798, 125-841, 125-887, 125-895, 125-901, 125-902, 125-908, 125-910, 125-916, 125-917, |
| | 126-879, 126-887, 131-262, 137-633, 141-399, 142-399, |
| 25 | 142-851, 157-841, 158-417, 158-645, 159-430, 160-384, 162-274, 162-437, 162-495, 162-556, 162-586, 162-592, |
| | 162-605, 162-662, 163-408, 164-275, 164-409, 164-450, 165-464, 165-872, 165-879, 168-412, 168-592, 170-422, |
| | 170-430, 170-432, 170-655, 172-299, 175-412, 175-421, 175-468, 175-480, 176-430, 176-613, 197-879, 211-871, |
| | 219-473, 220-546, 221-842, 232-558, 243-482, 268-491, 269-329, 269-376, 270-520, 276-658, 282-833, 296-409, |
| | 316-479, 334-541, 353-883, 355-724, 359-905, 360-603, 360-645, 374-855, 376-817, 377-589, 380-549, 380-602, |
| | 400-672, 400-827, 470-844, 502-788, 502-872, 508-864, 544-777, 619-866, 621-867, 631-892, 631-917, 636-867, |
| | 639-925, 666-925 |
| 26/3254315CB1/ | 1-598, 15-599, 48-696, 48-1284, 51-761, 58-670, 265-945, 273-915, 409-663, 424-950, 437-1255, 480-717, 480-960, |
| 7355 | 480-986, 484-821, 515-598, 840-938, 985-1135, 989-1543, 1068-1814, 1225-1519, 1290-2056, 1362-2020, |
| | 1362-2085, 1362-2118, 1362-2128, 1383-2205, 1438-2119, 1481-2792, 1492-2119, 1541-2395, 1578-2395, 1590-2154, |
| | 1590-2159, 1592-2159, 1672-1961, 1703-1920, 1703-2278, 1703-2395, 1715-2391, 1796-7339, 1873-2404, |
| | 1873-2566, 1927-2583, 1954-2395, 2002-2683, 2005-2579, 2006-2589, 2025-2564, 2033-2683, 2049-2720, 2054-2559, |
| | 2061-2691, 2064-2685, 2088-2375, 2153-2734, 2155-2459, 2161-2766, 2163-2685, 2173-2702, 2178-2674, |
| | 2189-2692, 2235-2885, 2239-2653, 2257-2543, 2273-2939, 2292-2778, 2295-2716, 2296-2815, 2302-2818, 2304-2872, |
| | 2313-2928, 2315-2718, 2315-2827, 2332-3048, 2382-2664, 2382-2932, 2383-2920, 2420-2956, 2428-2745, |
| | 2434-3093, 2468-2674, 2485-2723, 2504-3031, 2541-3020, 2542-7355, 2584-2758, 2652-3083, 2663-3447, |
| | 2668-3095, 2679-3120, 2681-3250, 2681-3401, 2704-3302, 2761-3017, 2792-2893, 2800-3028, 2801-3036, 2804-3234, |
| | 2824-3418, 2826-3475, 2829-3525, 2831-2996, 2846-3506, 2855-3108, 2855-3383, 2875-3383, 2881-3394, |
| | 2884-3163, 2926-3446, 2944-3631, 2954-3511, 2954-3589, 2954-3685, 2956-3440, 2971-3228, 2971-3467, 3018-3607, |
| | 3041-3714, 3058-3422, 3070-3320, 3082-3624, 3082-3687, 3085-3604, 3126-3714, 3127-3734, 3129-3645, |
| | 3148-3509, 3189-3432, 3196-3700, 3197-3755, 3241-3821, 3393-3434, 3477-3599 |
| 27/7472707CB1/ | 1-246, 5-246, 69-1010, 696-836, 696-860, 696-1433, 699-860, 714-860, 815-860, 861-1010, 1307-1527, 1434-1706, |
| 3369 | 1632-1825, 1632-2378, 1800-2540, 1800-2981, 1854-2281, 1854-2333, 2252-2512, 2297-2504, 2298-2508, 2298-2771, |
| | 2685-3351, 2884-3369, 2895-3369, 2911-3360, 2922-3360, 2923-3369 |
| 28/7480432CB1/ | 1-381, 4-540, 25-313, 29-51, 29-421, 30-51, 31-421, 469-490 |
| 540 | |
| 29/7494181CB1/ | 1-1070, 230-1018, 923-1288, 923-1290, 926-1290, 1151-1618, 1151-1621, 1151-1622, 1151-1625, 1151-1627, 1151-1630, |
| 5454 | 1151-1631, 1154-1698, 1203-1631, 1207-1622, 1245-1631, 1258-1627, 1554-1751, 1554-1826, 1554-2081, |
| | 1554-2296, 1554-2303, 1554-2333, 1554-2343, 1556-2370, 1565-2307, 1655-2451, 1771-3209, 1910-2508, 2099-2566, |
| | 2190-2380, 2331-2852, 2422-3209, 2506-3311, 2528-3311, 2534-3311, 2815-3433, 2831-3505, 2873-3600, |
| | 2881-3284, 2881-3311, 2884-3311, 2887-3505, 2921-3505, 2928-3505, 2940-3505, 2968-3311, 2981-3505, 3101-3209, |
| | 3198-3505, 3264-4176, 3718-4214, 3718-4215, 3718-4216, 3983-4560, 4023-4216, 4077-4890, 4356-4760, |
| | 4356-5124, 4356-5188, 4356-5193, 4362-4760, 4363-4760, 4385-4760, 4429-4760, 4473-4760, 4554-5392, 4589-4819, |
| | 4589-5048, 4599-5313, 4700-5454, 4752-4890, 4756-4890, 4757-5454, 4835-5433, 4862-5081, 4958-5212, |
| | 5001-5257 |
| 30/3697053CB1/ | 1-1050, 923-1183, 1064-1463, 1183-1400, 1183-1595, 1183-1623, 1183-1636, 1183-1639, 1183-1648, 1183-1661, |
| 3670 | 1183-1732, 1183-1744, 1183-1750, 1183-1795, 1183-1802, 1183-1807, 1183-1812, 1183-1897, 1237-1750, 1282-1839, |
| | 1344-2015, 1397-2062, 1421-1644, 1421-1651, 1473-2002, 1518-1788, 1518-1971, 1548-2287, 1557-1982, |
| | 1577-1913, 1577-1915, 1601-2162, 1601-2196, 1641-2357, 1643-2238, 1657-2116, 1737-2360, 1761-2377, 1761-2423, |
| | 1780-2488, 1790-2238, 1791-2489, 1833-2446, 1838-2226, 1838-2423, 1839-2418, 1851-2558, 1866-2409, |
| | 1915-2504, 1969-2618, 1987-2638, 1992-2546, 1993-2512, 2013-2584, 2054-2441, 2126-2680, 2162-2689, 2187-2735, |
| | 2215-2906, 2251-2467, 2282-2803, 2332-2873, 2370-2852, 2419-3114, 2429-3063, 2434-3060, 2574-3203, |
| | 2590-3262, 2603-3183, 2612-2883, 2612-3135, 2634-2858, 2675-2942, 2736-3406, 2754-3411, 2788-3270, 2866-3541, |
| | 2906-3557, 3000-3642, 3166-3670, 3173-3670, 3230-3670, 3255-3670, 3268-3670, 3305-3584, 3444-3652 |
| 31/7473203CB1/ | 1-1008, 235-460, 235-692, 235-1005, 235-1008, 461-660, 461-1009, 661-795, 796-1005 |
| 1009 | |
| 32/4697002CB1/ | 1-659, 168-1074, 465-661, 668-1037, 715-1147, 809-1076, 965-1257, 965-1457, 968-1434, 969-1458, 969-1498, |
| 2398 | 969-1509, 973-1492, 973-1501, 1019-1597, 1050-1670, 1057-1532, 1076-1290, 1080-1670, 1152-1413, 1152-1726, |
| | 1156-1420, 1156-1676, 1156-1709, 1175-1685, 1239-1787, 1256-2048, 1304-1680, 1364-1994, 1437-1687, 1464-2008, |
| | 1469-2080, 1531-2079, 1551-2077, 1564-1865, 1564-2160, 1662-2116, 1696-2008, 1708-2114, 1737-2117, |
| | 1741-2398, 1748-2304, 1777-2080, 1780-2073, 1892-2305, 1896-2308, 1950-2384, 1980-2206, 1980-2398, 1983-2398 |
| 33/5632139CB1/ | 1-210, 1-240, 1-439, 1-470, 1-508, 8-210, 9-405, 66-283, 84-210, 99-572, 236-500, 239-875, 414-873, 415-870, 415-872, |
| 4160 | 415-873, 558-988, 710-873, 787-1346, 847-1102, 847-1185, 847-1321, 847-1446, 862-1394, 899-1644, 949-1608, |
| | 956-1630, 1048-1299, 1066-1397, 1080-1716, 1081-1670, 1126-1704, 1148-1794, 1199-1819, 1221-1779, |
| | 1247-1479, 1285-1485, 1305-1935, 1336-1964, 1345-1968, 1352-1466, 1352-1959, 1353-1513, 1353-2049, 1363-2025, |
| | 1363-2049, 1378-1968, 1398-1968, 1403-1968, 1425-1940, 1431-1964, 1431-1969, 1436-1967, 1445-1700, |
| | 1446-1968, 1459-1968, 1474-1762, 1482-1954, 1493-1936, 1506-1968, 1507-1968, 1538-1968, 1545-1689, 1550-1968, |
| | 1561-1968, 1564-1968, 1579-1968, 1646-2158, 1650-2242, 1691-2244, 1716-1970, 1725-2337, 1726-1970, |
| | 1800-2438, 1824-2389, 1922-2508, 1971-2225, 1971-2324, 1971-2508, 2055-2307, 2186-2657, 2207-2749, 2222-2483, |
| | 2222-2616, 2222-2695, 2222-2712, 2222-2728, 2222-2756, 2222-2765, 2222-2769, 2222-2773, 2222-2787, |
| | 2222-2790, 2222-2808, 2222-2821, 2222-2892, 2224-2844, 2224-2864, 2224-3005, 2241-2488, 2242-2488, 2249-2904, |
| | 2269-2768, 2299-2886, 2301-2922, 2301-3007, 2306-2886, 2309-2952, 2315-2985, 2332-2664, 2332-2749, |
| | 2342-2611, 2371-2843, 2374-3033, 2376-3004, 2376-3012, 2384-2594, 2396-3251, 2400-2872, 2400-2890, 2407-2769, |
| | 2415-2625, 2415-2780, 2432-3091, 2436-2642, 2436-2892, 2463-2942, 2463-3024, 2484-2828, 2489-2752, |
| | 2530-3152, 2533-3261, 2535-3018, 2538-3068, 2539-2847, 2541-3222, 2545-3123, 2546-2665, 2546-2705, 2548-3204, |
| | 2553-3212, 2560-3228, 2560-3371, 2571-3183, 2580-3213, 2586-3187, 2587-3244, 2587-3459, 2596-3208, |
| | 2610-3230, 2623-3007, 2625-2845, 2628-3148, 2641-3221, 2649-3220, 2667-3367, 2684-3310, 2687-3348, 2698-3291, |
| | 2706-3235, 2717-3249, 2817-3485, 2823-3496, 2828-3504, 2829-3479, 2858-3532, 2917-3510, 2920-3191, |
| | 2922-3216, 2925-3427, 2929-3533, 2937-3585, 2941-3494, 2941-3662, 2945-3661, 2956-3496, 2977-3755, |
| | 2982-3561, 2990-3542, 2995-3387, 2995-3624, 2997-3248, 3009-3215, 3012-3216, 3016-3731, 3021-3640, 3027-3712, |
| | 3031-3747, 3040-3696, 3041-3288, 3041-3784, 3047-3534, 3063-3716, 3076-3639, 3079-3725, 3093-3643, |
| | 3109-3484, 3122-3784, 3125-3784, 3147-3682, 3147-3782, 3148-3725, 3151-3661, 3154-3650, 3180-3884, 3198-3780, |
| | 3204-3909, 3210-3847, 3213-3823, 3215-3886, 3218-3881, 3236-3872, 3238-3909, 3253-3783, 3255-3849, |
| | 3259-3869, 3261-3789, 3277-3871, 3278-3696, 3283-4009, 3295-3880, 3309-3854, 3311-3794, 3322-3955, 3324-3972, |
| | 3326-3690, 3333-3783, 3334-3960, 3342-3940, 3347-3953, 3349-3940, 3356-3918, 3358-3957, 3365-3676, |
| | 3365-3848, 3366-3734, 3375-3774, 3391-3803, 3392-3861, 3392-3918, 3394-4034, 3399-4050, 3409-3816, 3414-3960, |
| | 3436-3874, 3455-3948, 3465-3957, 3472-4072, 3477-3957, 3479-4020, 3489-3976, 3512-3539, 3512-3545, |
| | 3512-3550, 3512-3551, 3512-3559, 3512-3561, 3512-3562, 3512-3565, 3512-3578, 3512-3579, 3512-3581, |
| 33 | 3512-3585, 3512-3586, 3512-3587, 3512-3588, 3512-3593, 3512-3594, 3512-3596, 3512-3600, 3512-3602, 3512-3603, |
| | 3512-3612, 3512-3616, 3512-3624, 3512-3625, 3512-3627, 3512-3628, 3512-3639, 3512-3640, 3512-3649, |
| | 3512-3650, 3512-3651, 3512-3652, 3512-3653, 3512-3655, 3512-3657, 3512-3658, 3512-3659, 3512-3661, 3512-3665, |
| | 3512-3666, 3512-3669, 3512-3670, 3512-3681, 3512-3687, 3512-3688, 3512-3689, 3512-3692, 3512-3710, |
| | 3512-3716, 3512-3717, 3512-3718, 3512-3719, 3512-3723, 3512-3724, 3512-3725, 3512-3729, 3512-3731, 3512-3749, |
| | 3512-3751, 3512-3753, 3512-3758, 3512-3764, 3512-3769, 3512-3770, 3512-3771, 3512-3775, 3512-3776, |
| | 3512-3783, 3512-3788, 3512-3790, 3512-3799, 3512-3806, 3512-3808, 3512-3809, 3512-3818, 3512-3841, 3513-3776, |
| | 3514-3776, 3515-3561, 3515-3585, 3515-3587, 3515-3592, 3515-3603, 3515-3613, 3515-3622, 3515-3641, |
| | 3515-3644, 3516-4070, 3520-3710, 3521-3644, 3522-3776, 3522-3976, 3522-4076, 3523-3586, 3526-3776, |
| | 3527-3776, 3528-3776, 3529-4093, 3532-3770, 3532-4135, 3545-3641, 3545-3687, 3547-4153, 3548-3818, 3548-3841, |
| | 3551-3841, 3554-3692, 3563-3776, 3564-3980, 3566-4148, 3570-3841, 3570-3972, 3572-3841, 3578-3603, |
| | 3578-3650, 3578-3668, 3578-3693, 3578-3709, 3578-3716, 3578-3727, 3578-3731, 3578-3748, 3578-3756, 3578-3758, |
| | 3578-3790, 3578-3796, 3578-3803, 3578-3804, 3578-3809, 3578-3819, 3578-3820, 3578-3823, 3578-3848, |
| | 3578-3849, 3578-3850, 3578-3855, 3578-3875, 3578-3882, 3578-3884, 3578-3886, 3578-3887, 3578-3907, 3581-3907, |
| | 3582-3907, 3588-3907, 3595-3884, 3597-3898, 3600-3687, 3601-4160, 3608-4146, 3612-4102, 3614-3771, |
| | 3614-3818, 3614-3841, 3614-3907, 3617-3841, 3626-4160, 3630-3972, 3635-3841, 3635-3907, 3637-4136, 3639-3841, |
| | 3643-3692, 3643-3716, 3643-3755, 3643-3770, 3643-3771, 3643-3781, 3643-3792, 3643-3799, 3643-3821, |
| | 3643-3824, 3643-3843, 3643-3847, 3643-3856, 3643-3862, 3643-3865, 3643-3866, 3643-3878, 3643-3902, |
| | 3643-3903, 3643-3908, 3645-3909, 3646-3908, 3647-3908, 3653-3908, 3653-4146, 3657-4135, 3659-3929, 3660-3911, |
| | 3663-3908, 3676-3771, 3676-3818, 3678-3907, 3679-3907, 3694-3972, 3695-3908, 3697-3908, 3701-3907, |
| | 3701-3908, 3703-3907, 3709-3758, 3709-3820, 3709-3825, 3709-3840, 3709-3847, 3709-3858, 3709-3867, 3709-3878, |
| | 3709-3882, 3709-3884, 3709-3907, 3712-3907, 3713-3907, 3719-3907, 3725-3907, 3726-3884, 3728-3908, |
| | 3733-3818, 3733-3908, 3738-3907, 3741-3908, 3742-3907, 3742-4155, 3744-3907, 3745-3908, 3760-3908, 3761-3908, |
| | 3766-3907, 3767-3882, 3777-3823, 3777-3847, 3777-3865, 3777-3902, 3777-3903, 3777-3904, 3777-3906, |
| | 3777-3907, 3778-3907, 3779-3907, 3781-3865, 3784-3907, 3788-3907, 3790-3908, 3791-3907, 3793-3907, 3794-3907, |
| | 3794-3908, 3796-3943, 3797-3907, 3804-3907, 3811-3907, 3813-3908, 3822-3863, 3829-3907, 3829-3908, |
| | 3832-3907, 3840-3867, 3840-3877, 3840-3878, 3840-3907, 3843-3907, 3850-3907 |
| 34/7506184CB1/ | 1-437, 1-2835, 4-732, 4-821, 207-348, 228-757, 281-606, 286-1047, 350-784, 350-931, 359-454, 359-605, 359-624, |
| 2835 | 362-1114, 386-891, 392-973, 393-941, 397-670, 399-936, 399-1126, 400-1126, 407-965, 736-1282, 966-1388, 998-1307, |
| | 1038-1252, 1038-1509, 1076-1348, 1099-1726, 1108-1350, 1113-1314, 1121-1375, 1143-1655, 1199-1563, |
| | 1208-1783, 1227-1588, 1263-1860, 1272-1549, 1277-1880, 1289-1542, 1289-1795, 1289-1818, 1289-1825, 1289-1850, |
| | 1291-1882, 1301-1523, 1301-1785, 1301-1839, 1301-1873, 1301-1904, 1304-1900, 1306-1877, 1306-2114, |
| | 1335-1625, 1349-1869, 1353-1643, 1361-1815, 1372-1618, 1390-1683, 1397-1687, 1422-1651, 1478-1960, 1510-1852, |
| | 1534-1780, 1575-1856, 1604-1857, 1676-1811, 1753-2019, 1808-2501, 1877-2467, 1879-2160, 1879-2494, |
| | 1880-2394, 1882-2466, 1888-2162, 1896-2442, 1899-2512, 1903-2463, 1906-2132, 1906-2158, 1919-2427, 1924-2191, |
| | 1924-2476, 1935-2462, 1937-2233, 1937-2254, 1957-2346, 1958-2090, 1958-2116, 1958-2202, 1958-2206, |
| | 1958-2224, 1986-2430, 1996-2359, 2003-2225, 2012-2353, 2013-2254, 2032-2480, 2041-2304, 2047-2290, 2053-2534, |
| | 2057-2484, 2060-2407, 2061-2497, 2077-2401, 2078-2532, 2083-2480, 2092-2352, 2093-2466, 2093-2470, |
| | 2093-2474, 2093-2480, 2093-2485, 2095-2470, 2102-2479, 2107-2505, 2117-2482, 2126-2329, 2126-2478, 2127-2488, |
| | 2129-2473, 2129-2480, 2146-2500, 2149-2377, 2149-2462, 2149-2475, 2150-2359, 2151-2480, 2167-2517, |
| | 2177-2427, 2188-2480, 2188-2481, 2205-2475, 2210-2474, 2210-2480, 2218-2528, 2228-2539, 2241-2478, 2251-2490, |
| | 2251-2497, 2255-2406, 2261-2517, 2261-2528, 2292-2528, 2301-2496, 2333-2495, 2356-2479, 2363-2480, |
| | 2406-2478 |

TABLE 5

| Polynucleotide | Incyte | Representative |
|---|---|---|
| SEQ ID NO: | Project ID: | Library |
| 18 | 551243CB1 | BRABDIR01 |
| 19 | 7493587CB1 | STOMTUT02 |
| 20 | 4505840CB1 | UTRCDIE01 |
| 22 | 3559054CB1 | BRSTNOT01 |
| 23 | 7477526CB1 | PROSTUS19 |
| 24 | 7487253CB1 | LUNGNOT38 |
| 25 | 2131556CB1 | THYRTUT03 |
| 26 | 3254315CB1 | LUNGTUT07 |
| 27 | 7472707CB1 | LIVRFEE02 |
| 29 | 7494181CB1 | LNODNOT03 |
| 30 | 3697053CB1 | SININOT05 |
| 32 | 4697002CB1 | DRGTNOT01 |
| 33 | 5632139CB1 | LUNGFET03 |
| 34 | 7506184CB1 | EPIPNON05 |

TABLE 6

| Library | Vector | Library Description |
|---|---|---|
| BRABDIR01 | pINCY | Library was constructed using RNA isolated from diseased cerebellum tissue removed from the brain of a |
| | | 57-year-old Caucasian male, who died from a cerebrovascular accident. Patient history included |
| | | Huntington's disease, emphysema, and tobacco abuse. |
| BRSTNOT01 | PBLUESCRIPT | Library was constructed using RNA isolated from the breast tissue of a 56-year-old Caucasian female who died in |
| | | a motor vehicle accident. |
| DRGTNOT01 | pINCY | Library was constructed using RNA isolated from dorsal root ganglion tissue removed from the thoracic spine |
| | | of a 32-year-old Caucasian male who died from acute pulmonary edema and bronchopneumonia, bilateral |
| | | pleural and pericardial effusions, and malignant lymphoma (natural killer cell type). Patient history |
| | | included probable cytomegalovirus infection, hepatic congestion and steatosis, splenomegaly, hemorrhagic |
| | | cystitis, thyroid hemorrhage, and Bell's palsy. Surgeries included colonoscopy, large intestine |
| | | biopsy, adenotonsillectomy, and nasopharyngeal endoscopy and biopsy; treatment included radiation |
| | | therapy. |
| EPIPNON05 | pINCY | This normalized prostate epithelial cell tissue library was constructed from 2.36 million independent |
| | | clones from a prostate epithelial cell tissue library. Starting RNA was made from untreated prostatic |
| | | epithelial cell issue removed from a 17-year-old Hispanic male. The library was normalized |
| | | in two rounds using conditions adapted from Soares et al., PNAS (1994) 91: 9228 and Bonaldo et al., |
| | | Genome Research (1996) 6: 791, except that a significantly longer (48-hours/round) reannealing |
| | | hybridization was used. |
| LIVRFEE02 | pINCY | This 5′ biased random primed library was constructed using RNA isolated from liver tissue removed from a |
| | | Caucasian male fetus who died from fetal demise. Serologies were negative. |
| LNODNOT03 | pINCY | Library was constructed using RNA isolated from lymph node tissue obtained from a 67-year-old Caucasian male |
| | | during a segmental lung resection and bronchoscopy. On microscopic exam, this tissue was found to be |
| | | extensively necrotic with 10% viable tumor. Pathology for the associated tumor tissue indicated invasive |
| | | grade 3-4 squamous cell carcinoma. Patient history included hemangioma. Family history included |
| | | atherosclerotic coronary artery disease, benign hypertension, congestive heart failure, |
| | | atherosclerotic coronary artery disease. |
| LUNGFET03 | pINCY | Library was constructed using RNA isolated from lung tissue removed from a Caucasian female fetus, who |
| | | died at 20 weeks' gestation. |
| LUNGNOT38 | pINCY | Library was constructed using RNA isolated from diseased lung tissue removed from a 15-year-old Caucasian |
| | | male who died from a gunshot wound to the head. Serology was positive for cytomegalovirus. Patient history |
| | | included asthma. |
| LUNGTUT07 | pINCY | Library was constructed using RNA isolated from lung tumor tissue removed from the upper lobe of |
| | | a 50-year-old Caucasian male during segmental lung resection. Pathology indicated an invasive grade 4 |
| | | squamous cell adenocarcinoma. Patient history included tobacco use. Family history included skin cancer. |
| PROSTUS19 | pINCY | This subtracted prostate tumor tissue library was constructed using 2.36 million clones from |
| | | a prostate tumor library and was subjected to two rounds of subtraction hybridization with 2.36 |
| | | million clones from a prostate epithelium library. The starting library for subtraction |
| | | was constructed using RNA isolated from prostate tumor tissue removed from a 59-year-old |
| | | Caucasian male during a radical prostatectomy with regional lymph node excision. Pathology |
| | | indicated adenocarcinoma (Gleason grade 3 + 3) involving the prostate peripherally |
| | | with invasion of the capsule. Adenofibromatous hyperplasia was present. The patient presented |
| | | with elevated prostate-specific antigen (PSA). Patient history included diverticulitis of colon, |
| | | asbestosis, and thrombophlebitis. Family history included benign hypertension, multiple myeloma, |
| | | hyperlipidemia, and rheumatoid arthritis. Subtractive hybridization conditions were based on |
| | | the methodologies of Swaroop et al., NAR (1991) 19: 1954 and Bonaldo, et al. Genome |
| | | Research (1996) 6: 791. |
| SININOT05 | pINCY | Library was constructed using RNA isolated from ileum tissue obtained from a 30-year-old Caucasian female |
| | | during partial colectomy, open liver biopsy, incidental appendectomy, and permanent colostomy. |
| | | Patient history included endometriosis. Family history included hyperlipidemia, anxiety, |
| | | and upper lobe lung cancer, stomach cancer, liver cancer, and cirrhosis. |
| STOMTUT02 | pINCY | Library was constructed using RNA isolated from stomach tumor tissue obtained from a 68-year-old |
| | | Caucasian female during a partial gastrectomy. Pathology indicated a malignant lymphoma of |
| | | diffuse large-cell type. Previous surgeries included cholecystectomy. Patient history included |
| | | thalassemia. Family history included acute leukemia, malignant neoplasm of the esophagus, |
| | | malignant stomach neoplasm, and atherosclerotic coronary artery disease. |
| THYRTUT03 | pINCY | Library was constructed using RNA isolated from benign thyroid tumor tissue removed from a 17-year-old |
| | | Caucasian male during a thyroidectomy. Pathology indicated encapsulated follicular adenoma forming a |
| | | circumscribed mass. |
| UTRCDIE01 | PCDNA2.1 | This 5′ biased random primed library was constructed using RNA isolated from uterine cervix tissue removed |
| | | from a 29-year-old Caucasian female during a vaginal hysterectomy and cystocele repair. Pathology indicated the |
| | | cervix showed mild chronic cervicitis with focal squamous metaplasia. Pathology for the matched tumor tissue |
| | | indicated intramural uterine leiomyoma. Patient history included hypothyroidism, pelvic floor relaxation, paraplegia, |
| | | and self catheterization. Previous surgeries included a normal delivery, a laminectomy, and a rhinoplasty. Patient |
| | | medications included Synthroid. Family history included benign hypertension in the father; and type II |
| | | diabetes and hyperlipidemia in the mother. |

TABLE 7

| Program | Description | Reference | Parameter Threshold |
|---|---|---|---|
| ABI | A program that removes vector sequences | Applied Biosystems, Foster City, CA. | |
| FACTURA | and masks ambiguous bases in nucleic | | |
| | acid sequences. | | |
| ABI/ | A Fast Data Finder useful in comparing | Applied Biosystems, Foster City, CA; | Mismatch <50% |
| PARACEL | and annotating amino acid or nucleic acid | Paracel Inc., Pasadena, CA. | |
| FDF | sequences. | | |
| ABI | A program that assembles nucleic acid | Applied Biosystems, Foster City, CA. | |
| AutoAssembler | sequences. | | |
| BLAST | A Basic Local Alignment Search Tool useful | Altschul, S. F. et al. (1990) J. Mol. Biol. | ESTs: Probability value = 1.0E−8 |
| | in sequence similarity search for amino | 215: 403-410; Altschul, S. F. et al. (1997) | or less |
| | acid and nucleic acid sequences. BLAST | Nucleic Acids Res. 25: 3389-3402. | Full Length sequences: Probability |
| | includes five functions: blastp, blastn, | | value = 1.0E−10 or less |
| | blastx, tblastn, and tblastx. | | |
| FASTA | A Pearson and Lipman algorithm that | Pearson, W. R. and D. J. Lipman (1988) Proc. | ESTs: fasta E value = 1.06E−6 |
| | searches for similarity between a query | Natl. Acad Sci. USA 85: 2444-2448; Pearson, | Assembled ESTs: fasta Identity = 95% |
| | sequence and a group of sequences | W. R. (1990) Methods Enzymol. 183: 63-98; | or greater and |
| | of the same type. FASTA comprises as | and Smith, T. F. and M. S. Waterman (1981) | Match length = 200 bases or greater; |
| | least five functions: fasta, tfasta, fastx, | Adv. Appl. Math. 2: 482-489. | fastx E value = 1.0E−8 or less |
| | tfastx, and ssearch. | | Full Length sequences: |
| | | | fastx score = 100 or greater |
| BLIMPS | A BLocks IMProved Searcher that matches | Henikoff, S. and J. G. Henikoff (1991) Nucleic | Probability value = 1.0E−3 or less |
| | a sequence against those in BLOCKS, | Acids Res. 19: 6565-6572; Henikoff, J. G. and | |
| | PRINTS, DOMO, PRODOM, and PFAM | S. Henikoff (1996) Methods | |
| | databases to search for gene families, | Enzymol. 266: 88-105; and Attwood, T. K. et | |
| | sequence homology, and structural | al. (1997) J. Chem. Inf. Comput. Sci. 37: | |
| | fingerprint regions. | 417-424. | |
| HMMER | An algorithm for searching a query | Krogh, A. et al. (1994) J. Mol. Biol. | PFAM, INCY, SMART, or TIGRFAM |
| | sequence against hidden Markov model | 235: 1501-1531; Sonnhammer, E. L. L. et al. | hits: Probability value = 1.0E−3 or less |
| | (HMM)-based databases of protein | (1988) Nucleic Acids Res. 26: 320-322; | Signal peptide hits: Score = 0 or |
| | family consensus sequences, such as PFAM, | Durbin, R. et al. (1998) Our World View, in a | greater |
| | INCY, SMART, and TIGRFAM. | Nutshell, Cambridge Univ. Press, pp. 1-350. | |
| ProfileScan | An algorithm that searches for structural | Gribskov, M. et al. (1988) CABIOS 4: 61-66; | Normalized quality score ≧ GCG- |
| | and sequence motifs in protein sequences | Gribskov, M. et al. (1989) Methods Enzymol. | specified “HIGH” value for that |
| | that match sequence patterns defined | 183: 146-159; Bairoch, A. et al. (1997) | particular Prosite motif. |
| | in Prosite. | Nucleic Acids Res. 25: 217-221. | Generally, score = 1.4-2.1. |
| Phred | A base-calling algorithm that examines | Ewing, B. et al. (1998) Genome Res. | |
| | automated sequencer traces with high | 8: 175-185; Ewing, B. and P. Green | |
| | sensitivity and probability. | (1998) Genome Res. 8: 186-194. | |
| Phrap | A Phils Revised Assembly Program | Smith, T. F. and M. S. Waterman (1981) Adv. | Score = 120 or greater; |
| | including SWAT and CrossMatch, programs | Appl. Math. 2: 482-489; Smith, T. F. and | Match length = 56 or greater |
| | based on efficient implementation of the | M. S. Waterman (1981) J. Mol. Biol. 147: | |
| | Smith-Waterman algorithm, useful in | 195-197; and Green, P., University of | |
| | searching sequence homology and | Washington, Seattle, WA. | |
| | assembling DNA sequences. | | |
| Consed | A graphical tool for viewing and editing | Gordon, D. et al. (1998) Genome Res. | |
| | Phrap assemblies. | 8: 195-202. | |
| SPScan | A weight matrix analysis program that | Nielson, H. et al. (1997) Protein Engineering | Score = 3.5 or greater |
| | scans protein sequences for the presence | 10: 1-6; Claverie, J. M. and S. Audic (1997) | |
| | of secretory signal peptides. | CABIOS 12: 431-439. | |
| TMAP | A program that uses weight matrices to | Persson, B. and P. Argos (1994) J. Mol. Biol. | |
| | delineate transmembrane segments on | 237: 182-192; Persson, B. and P. Argos (1996) | |
| | protein sequences and determine | Protein Sci. 5: 363-371. | |
| | orientation. | | |
| TMHMMER | A program that uses a hidden Markov | Sonnhammer, E. L. et al. (1998) Proc. Sixth | |
| | model (HMM) to delineate transmembrane | Intl. Conf. on Intelligent Systems for Mol. | |
| | segments on protein sequences and | Biol., Glasgow et al., eds., The Am. Assoc. | |
| | determine orientation. | for Artificial Intelligence Press, Menlo Park, | |
| | | CA, pp. 175-182. | |
| Motifs | A program that searches amino acid | Bairoch, A. et al. (1997) Nucleic Acids | |
| | sequences for patterns that matched | Res. 25: 217-221; Wisconsin Package Program | |
| | those defined in Prosite. | Manual, version 9, page M51-59, Genetics | |
| | | Computer Group, Madison, WI. | |

1. An isolated polypeptide selected from the group consisting of:
a) a polypeptide comprising an amino acid sequence selected from the group consisting of SEQ ID NO:1-17,
b) a polypeptide comprising a naturally occurring amino acid sequence at least 90% identical to an amino acid sequence selected from the group consisting of SEQ ID NO:1-2, SEQ ID NO:4-8, and SEQ ID NO:10-15,
c) a polypeptide comprising a naturally occurring amino acid sequence at least 91% identical to an amino acid sequence selected from the group consisting of consisting of SEQ ID NO:3 and SEQ ID NO:9,
d) a polypeptide comprising a naturally occurring amino acid sequence at least 94% identical to an amino acid sequence consisting of SEQ ID NO:16,
e) a polypeptide comprising a naturally occurring amino acid sequence at least 93%. identical to an amino acid sequence consisting of SEQ ID NO:17,
f) a biologically active fragment of a polypeptide having an amino acid sequence selected from the group consisting of SEQ ID NO:1-17, and
g) an immunogenic fragment of a polypeptide having an amino acid sequence selected from the group consisting of SEQ ID NO:1-17.

2. An isolated polypeptide of claim 1 comprising an amino acid sequence selected from the group consisting of SEQ ID NO:1-17.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:18-34.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-17.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:18-34,
b) a polynucleotide comprising a naturally occurring polynucleotide sequence at least 90% identical to a polynucleotide sequence selected from the group consisting of SEQ ID NO:18-19, SEQ ID NO:21-25, and SEQ ID NO:27-34,
c) a polynucleotide comprising a naturally occurring polynucleotide sequence at least 91% identical to a polynucleotide sequence selected from the group consisting of SEQ ID NO:20 and SEQ ID NO:26,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-17.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridization nucleic acids of the treated biological same with a probe comprising at least 20 contagious nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30.-89. (canceled)


SEQUENCE LISTING (34 sequences)

SEQ ID NO: 1 - protein, 547 aa, Homo sapiens
  misc_feature: Incyte ID No 551243CD1
MEVEEAFQAVGEMGIYQMYLCFLLAVLLQLYVATEAILIALVGATPSYHWDLAELLPNQSHGNQSAGEDQAFGDWLLTANGSEIHKHVHFSSSFTSIASEWFLIANRSYKVSAASSFFFSGVFVGVISFGQLSDRFGRKKVYLTGFALDILFAIANGFSPSYEFFAVTRFLVGMMNGGMSLVAFVLLNECVGTAYWALAGSIGGLFFAVGIAQYALLGYFIRSWRTLAILVNLQGTVVFLLSLFIPESPRWLYSQGRLSEAEEALYLIAKRNRKLKCTFSLTHPANRSCRETGSFLDLFRYRVLLGHTLILMFIWFVCSLVYYGLTLSAGDLGGSIYANLALSGLIEIPSYPLCIYLINQKWFGRKRTLSAFLCLGGLACLIVMFLPEKKDTGVFAVVNSHSLSLLGKLTISAAFNIVYIYTSELYPTVIRNVGLGTCSMFSRVGGIIAPFIPSLKYVQWSLPFIVFGATGLTSGLLSLLLPETLNSPLLETFSDLQVYSYRRLGEEALSLQALDPQQCVDKESSLGSESEEEEEFYDADEETQMIK

SEQ ID NO: 2 - protein, 1499 aa, Homo sapiens
  misc_feature: Incyte ID No 7493587CD1
MEREPAGTEEPGPPGRRRRREGRTRTVRSNLLPPPGAEDPAAGAAKGERRRRRGCAQHLADNRLKTTKYTLLSFLPKNLFEQFHRPANVYFVFIALLNFVPAVNAFQPGLALAPVLFILAITAFRDLWEDYSRHRSDHKINHLGCLVFSREEKKYVNRFWKEIHVGDFVRLRCNEIFPADILLLSSSDPDGLCHIETANLDGETNLKRRQVVRGFSELVSEFNPLTFTSVIECEKPNNDLSRFRGCIIHDNGKKAGLYKENLLLRGCTLRNTDAVVGIVIYAGHETKALLNNSGPRYKRSKLERQMNCDVLWCVLLLVCMSLFSAVGHGLWIWRYQEKKSLFYVPKSDGSSLSPVTAAVYSFLTMIIVLQVLIPISLYVSIEIVKACQVYFINQDMQLYDEETDSQLQCRALNITEDLGQIQYIFSDKTGTLTENKMVFRRCTVSGVEYSHDANAQRLARYQEADSEEEEVVPRGGSVSQRGSIGSHQSVRVVHRTQSTKSHRRTGSRAEAKRASMLSKHTAFSSPMEKDITPDPKLLEKVSECDKSLAVARHQEHLLAHLSPELSDVFDFFIALTICNTVVVTSPDQPRTKVRVRFELKSPVKTIEDFLRRFTPSCLTSGCSSIGSLAANKSSHKLGSSFPSTPSSDGMLLRLEERLGQPTSAIASNGYSSQADNWASELAQEQESERELRYEAESPDEAALVYAARAYNCVLVERLHDQVSVELPHLGRLTFELLHTLGFDSVRKRMSVVIRHPLTDEINVYTKGADSVVMDLLQPCSSVDARGRHQKKIRSKTQNYLNVYAAEGLRTLCIAKRVLSKEEYACWLQSHLEAESSLENSEELLFQSAIRLETNLHLLGATGIEDRLQDGVPETISKLRQAGLQIWVLTGDKQETAVNIAYACKLLDHDEEVITLNATSQEACAALLDQCLCYVQSRGLQRAPEKTKGKVSMRFSSLCPPSTSTASGRRPSLVIDGRSLAYALEKNLEDKFLFLAKQCRSVLCCRSTPLQKSMVVKLVRSKLKAMTLAIGDGANDVSMIQVADVGVGISGQEGMQAVMASDFAVPKFRYLERLLILHGHWCYSRLANMVLYFFYKNTMFVGLLFWFQFFCGFSASTMIDQWYLIFFNLLFSSLPPLVTGVLDRDVPANVLLTNPQLYKSGQNMEEYRPRTFWFNMADATFQSLVCFSIPYLAYYDSNVDLFTWGTPIVTIALLTFLLHLGIETKTWTWLNWITCGFSVLLFFTVALIYNASCATCYPPSNPYWTMQALLGDPVFYLTCLMTPVAALLPRLFFRSLQGSVFPTQLQLARQLTRKSPRRCSAPKETFAQGRLPKDSGTEHSSGRTVKTSVPLSQPSWHTQQPVCSLEASGEPSTVDMSMPVREHTLLEGLSAPAPMSSAPGEAVLRSPGGCPEESKVRAASTGRVTPLSSLFSLPTFSLLNWISSWSLVSRLGSVLQFSRTEQLADGQAGRGLPVQPHSGRSGLQGPDHRLLIGASSRRSQ

SEQ ID NO: 3 - protein, 811 aa, Homo sapiens
  misc_feature: Incyte ID No 4505840CD1
MPKPPKPRNNLEDRHNPGIQGRREHRPGPGRVRAASSPGGSAPRAERRLWGEGWESGAAPHPHSSRVSALRPCGVVGAWVGMGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANEGFDPSTLSDPLYVLDQAVGAYLPLTTARYSALLAGNLRI

SEQ ID NO: 4 - protein, 545 aa, Homo sapiens
  misc_feature: Incyte ID No 7484873CD1
MLKQSERRRSWSYRPWNTTENEGSQHRRSICSLGARSGSQASIHGWTEGNYNYYIEEDEDGEEEDQWKDDLAEEDQQAGEVTTAKPEGPSDPPALLSTLNVNVGGHSYQLDYCELAGFPKTRLGRLATSTSRSRQLSLCDDYEEQTDEYFFDRDPAVFQLVYNFYLSGVLLVLDGLCPRRFLEELGYWGVRLKYTPRCCRTCFEERRDELSERLKIQHELRAQAQVEEAEELFRDMRFYGPQRRRLWNLMEKPFSSVAAKAIGVASSTFVLVSVVALALNTVEEMQQHSGQGEGGPDLRPILEHVEMLCMGFFTLEYLLRLASTPDLRRFARSALNLVDLVAILPLYLQLLLECFTGEGHQRGQTVGSVGKVGQVLRVMRLMRIFRILKLARHSTGLRAFGFTLRQCYQQVGCLLLFIAMGIFTFSAAVYSVEHDVPSTNFTTIPHSWWWAAVSISTVGYGDMYPETHLGRFFAFLCIAFGIILNGMPISILYNKFSDYYSKLKAYEYTTIRRERGEVNFMQRARKKIAECLLGSNPQLTPRQEN

SEQ ID NO: 5 - protein, 1583 aa, Homo sapiens
  misc_feature: Incyte ID No 3559054CD1
MNMKQKSVYQQTKALLCKNFLKKWRMKRESLLEWGLSILLGLCIALFSSSMRNVQFPGMAPQNLGRVDKFNSSSLMVVYTPISNLTQQIMNKTALAPLLKGTSVIGAPNKTHMDEILLENLPYAMGIIFNETFSYKLIFFQGYNSPLWKEDFSAHCWDGYGEFSCTLTKYWNRGFVALQTAINTAIIEITTNHPVMEELMSVTAITMKTLPFITKNLLHNEMFILFFLLHFSPLVYFISLNVTKERKKSKNLMKMMGLQDSAFWLSWGLIYAGFIFIISIFVTIIITFTQIIVMTGFMVIFILFFLYGLSLVALVFLMSVLLKKAVLTNLVVFLLTLFWGCLGFTVFYEQLPSSLEWILNICSPFAFTTGMIQIIKLDYNLNGVIFPDPSGDSYTMIATFSMLLLDGLIYLLLALYFDKILPYGDERHYSPLFFLNSSSCFQHQRTNAKVIEKEIDAEHPSDDYFEPVAPEFQGKEAIRIRNVKKEYKGKSGKVEALKGLLFDIYEGQITAILGHSGAGKSSLLNILNGLSVPTEGSVTIYNKNLSEMQDLEEIRKITGVCPQFNVQFDILTVKENLSLFAKIKGIHLKEVEQEILLLDEPTTGLDPFSRDQVWSLLRERRADHVILFSTQSMDEADILADRKVIMSNGRLKCAGSSMFLKRRWGLGYHLSLHRNEICNPEQITSFITHHIPDAKLKTENKEKLVYTLPLERTNTFPDLFSDLDKCSDQGVTGYDISMSTLNEVFMKLEGQSTIEQGKAICINFEQVEMIRDSESLNEMELAHSSFSEMQTAVSDMGLWRMQVFAMARLRFLKLKRQTKVLLTLLLVFGIAIFPLIVENIIYAMLNEKIDWEFKNELYFLSPGQLPQEPRTSLLIINNTESNIEDFIKSLKHQNILLEVDDFENRNGTDGLSYNGAIIVSGKQKDYRFSVVCNTKRLHCFPILMNIISNGLLQMFNHTQHIRIESSPFPLSHIGLWTGLPDGSFFLFLVLCSISPYITMGSISDYKKNAKSQLWISGLYTSAYWCGQALVDVSFFILILLLMYLIFYIENMQYLLITSQIVFALVIVTPGYAASLVFFIYMISFIFRKRRKNSGLWSFYFFFASTIMFSITLINHFDLSILITTMVLVPSYTLLGFKTFLEVRDQEHYREFPEANFELSATDFLVCFIPYFQTLLFVFVLRYMELKCGKKRMRKDPVFRISPQSRDAKPNPEEPIDEDEDIQTERIRTVTALTTSILDEKPVIIASCLHKEYAGQKKSCFSKRKKKIAARNISFCVQEGEILGLLGPSGAGKSSSIRMISGITKPTAGEVELKGCSSVLGHLGYCPQENVLWPMLTLREHLEVYAAVKGLREADARLAIARLVSAFKLHEQLNVPVQKLTAGITRKLCFVLSLLGNSPVLLLDEPSTGIDPTGQQQMWQAIQAVVKNTERGVLLTTHNLAEAEALCDRVAIMVSGRLRCIGSIQHLKNKLGKDYILELKVKETSQVTLVHTEILKLFPQAAGQERYSSLLTYKLPVADVYPLSQTFHKLEAVKHNFNLEEYSLSQCTLEKVFLELSKEQEVGNFDEEIDTTMRWKLLPHSDEP

SEQ ID NO: 6 - protein, 2004 aa, Homo sapiens
  misc_feature: Incyte ID No 7477526CD1
MIAPVTSQKSWIKGVFDKRECSTIIPSSKNPHRCYCGRLIGDHAGIDYSWTISAAKGKESEQWSVEKHTTKSPTDTFGTINFQDGEHTHHAKYIRTSYDTKLDHLLHLMLKEWKMELPKLVISVHGGIQNFTMPSKFKEIFSQGLVKAAETTGAWIITEGINTGVSKHVGDALKSHSSHSLRKIWTVGIPPWGVIENQRDLIGKDVVCLYQTLDNPLSKLTTLNSMHSHFILSDDGTVGKYGNEMKLRRNLEKYLSLQKIHCRSRQGVPVVGLVVEGGPNVILSVWETVKDKDPVVVCEGTGRAADLLAFTHKHLADEGMLRPQVKEEIICMIQNTFNFSLKQSKHLFQILMECMVHRDCITIFDADSEEQQDLDLAILTALLKGTNLSASEQLNLAMAWDRVDIAKKHILIYEQHWKPDALEQAMSDALVMDRVDFVKLLIEYGVNLHRFLTIPRLEELYNTKQGPTNTLLHHLVQDVKQHTLLSGYRITLIDIGLVVEYLIGRAYRSNYTRKHFRALYNNLYRKYKHQRHSSGNRNESAESTLHSQFIRTAQPYKFKEKSIVLHKSRKKSKEQNVSDDPESTGFLYPYNDLLVWAVLMKRQKMAMFFWQHGEEATVKAVIACILYRAMAHEAKESHMVDDASEELKNYSKQFGQLALDLLEKAFKQNERMAMTLLTYELRNWSNSTCLKLAVSGGLRPFVSHTCTQMLLTDMWMGRLKMRKNSWLKIIISIILPPTILTLEFKSKAEMSHVPQSQDFQFMWYYSDQNASSSKESASVKEYDLERGHDEKLDENQHFGLESGHQHLPWTRKVYEFYSAPIVKFWFYTMAYLAFLMLFTYTVLVEMQPQPSVQEWLVSIYIFTNAIEVVREICISEPGKFTQKVKVWISEYWNLTETVAIGLFSAGFVLRWGDPPFHTAGRLIYCIDIIFWFSRLLDFFAVNQHAGPYVTMIAKMTANMFYIVIIMAIVLLSFGVARKAILSPKEPPSWSLARDIVFEPYWMIYGEVYAGEIDVCSSQPSCPPGSFLTPFLQAVYLFVQYIIMVNLLIAFFNNVYLDMESISNNLWKYNRYRYIMTYHEKPWLPPPLILLSHVGLLLRRLCCHRAPHDQEEGDVGLKLYLSKEDLKKLHDFEEQCVEKYFHEKMEDVNCSCEERIRVTSERVTEMYFQLKEMNEKVSFIKDSLLSLDSQVGHLQDLSALTVDTLKVLSAVDTLQEDEALLAKRKHSTCKKLPHSWSNVICAEVLGSMEIAGEKKYQYYSMPSSLLRSLAGGRHPPRVQRGALLEITNSKREATNVRNDQERQETQSSIVVSGVSPNRQAHSKYGQFLLVPSNLKRVPFSAETVLPLSRPSVPDVLATEQDIQTEVLVHLTGQTPVVSDWASVDEPKEKHEPIAHLLDGQDKAEQVLPTLSCTPEPMTMSSPLSQAKIMQTGGGYVNWAFSEGDETGVFSIKKKWQTCLPSTCDSDSSRSEQHQKQAQDSSLSDNSTRSAQSSECSEVGPWLQPNTSFWINPLRRYRPFARSHSFRFHKEEKLMKICKIKNLSGSSEIGQGAWVKAKMLTKDRRLSKKKKNTQGLQVPIITVNACSQSDQLNPEPGENSISEEEYSKNWFTVSKFSHTGVEPYIHQKMKTKEIGQCAIQISDYLKQSQEDLSKNSLWNSRSTNLNRNSLLKSSIGVDKISASLKSPQEPHHHYSAIERNNLMRLSQTIPFTPVQLFAGEEITVYRLEESSPLNLDKSMSSWSQRGRAAMIQVLSREEMDGGLRKAMRVVSTWSEDDILKPGQVFIVKSFLPEVVRTWHKIFQESTVLHLCLREIQQQRAAQKLIYTFNQVKPQTIPYTPRFLEVFLIYCHSANQWLTIEKYMTGEFRKYNNNNGDEITPTNTLEELMLAFSHWTYEYTRGELLVLDLQGVGENLTDPSVIKPEVKQSRGMVFGPANLGEDAIRNFIAKHHCNSCCRKLKLPDLKRNDYSPERINSTFGLEIKIESAEEPPARETGRNSPEDDMQL

SEQ ID NO: 7 - protein, 281 aa, Homo sapiens
  misc_feature: Incyte ID No 7487253CD1
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKQEHINLSCDMHFEIAEPSIRGFLVLGYEGWLAGYQMNFETAKSQGTQSNFAVGYKTDEFQLHTNVNDGTEFGGSIYQKVNKKLESTVNLGWTAEKCKTCFEIAAKYQINPDACFLDKLNNFSLLGLGYIQTLKPGIRLTLSAFLYGKNVQAHKLDLRLEFQV

SEQ ID NO: 8 - protein, 236 aa, Homo sapiens
  misc_feature: Incyte ID No 2131556CD1
MAETKLQLFVKASEDGESVGHCPSCQRLFMVLLLKGVPFTLTTVDTRRSPDVLKDFAPGSQLPILLYDSDAKTDTLQIEDFLEETLGPPDFPSLAPRYRESNTAGNDVFHKFSAFIKNPVPAQDEALYQQLLRALARLDSYLRAPLEHELAGEPQLRESRRRFLDGDRLTLADCSLLPKLHIVDTVCAHFRQAPIPAELRGVRRYLDSAMQEKEFKYTCPHSAEILAAYRPAVHPR

SEQ ID NO: 9 - protein, 1177 aa, Homo sapiens
  misc_feature: Incyte ID No 3254315CD1
MWRWIRQQLGFDPPHQSDTRTIYVANRFPQNGLYTPQKFIDNRIISSKYTVWNFVPKNLFEQFRRVANFYFLIIFLVQLMIDTPTSPVTSGLPLFFVITVTAIKQGYEDWLRHNSDNEVNGAPVYVVRSGGLVKTRSKNIRVGDIVRIAKDEIFPADLVLLSSDRLDGSCHVTTASLDGETNLKTHVAVPETALLQTVANLDTLVAVIECQQPEADLYRFMGRMIITQQMEEIVRPLGPESLLLRGARLKNTKEIFGVAVYTGMETKMALNYKSKSQKRSAVEKSMNTFLIIYLVILISEAVISTILKYTWQAEEKWDEPWYNQKTEHQRNSSKILRFISDFLAFLVLYNFIIPISLYVTVEMQKFLGSFFIGWDLDLYHEESDQKAQVNTSDLNEELGQVEYVFTDKTGTLTENEMQFRECSINGMKYQEINGRLVPEGPTPDSSEGNLSYLSSLSHLNNLSHLTTSSSFRTSPENETELIKEHDLFFKAVSLCHTVQISNVQTDCTGDGPWQSNLAPSQLEYYASSPDEKALVEAAARIGIVFIGNSEETMEVKTLGKLERYKLLHILEFDSDRRRMSVIVQAPSGEKLLFAKGAESSILPKCIGGEIEKTRIHVDEFALKGLRTLCIAYRKFTSKEYEEIDKRIFEARTALQQREEKLAAVFQFIEKDLILLGATAVEDRLQDKVRETIEALRMAGIKVWVLTGDKHETAVSVSLSCGHFHRTMNILELINQKSDSECAEQLRQLARRITEDHVIQHGLVVDGTSLSLALREHEKLFMEVCRNCSAVLCCRMAPLQKAKVIRLIKISPEKPITLAVGDGANDVSMIQEAHVGIGIMGKEGRQAARNSDYAIARFKFLSKLLFVHGHFYYIRIATLVQYFFYKNVCFITPQFLYQFYCLFSQQTLYDSVYLTLYNICFTSLPILIYSLLEQHVDPHVLQNKPTLYRDISKNRLLSIKTFLYWTILGFSHAFIFFFGSYLLIGKDTSLLGNGQMFGNWTFGTLVFTVMVITVTVKMALETHFWTWINHLVTWGSIIFYFVFSLFYGGILWPFLGSQNMYFVFIQLLSSGSAWFAIILMVVTCLFLDIIKKVFDRHLHPTSTEKAQLTETNAGIKCLDSMCCFPEGEAACASVGRMLERVIGRCSPTHISRSWSASDPFYTNDRSILTLSTMDSSTC

SEQ ID NO: 10 - protein, 970 aa, Homo sapiens
  misc_feature: Incyte ID No 7472707CD1
MAQLERSAISGFSSKSRRNSFAYDVKREVYNEETFQQEHKRKASSSGNMNINITTFRHHVQCRCSWHRFLRCMLTIFPFLEWMCMYRLKDWLLGDLLAGISVGLVQVPQGLTLSLLARQLIPPLNIAYAAFCSSVIYVIFGSCHQMSIGSFFLVSALLINVLKVSPFNNGQLVMGSFVKNEFSAPSYLMGYNKSLSVVATTTFLTGIIQLIMGVLGLGFIATYLPESAMSAYLAAVALHIMLSQLTFIFGIMISFHAGPISFFYDIINYCVALPKANSTSILVFLTVVVALRINKCIRISFNQYPIEFPMELFLIIGFTVIANKISMATETSQTLIDMIPYSFLLPVTPDFSLLPKIILQAFSLSLVSSFLLIFLGKKIASLHNYSVNSNQDLIAIGLCNVVSSFFRSCVFTGAIARTIIQDKSGGRQQFASLVGAGVMLLLMVKMGHFFYTLPNAVLAGIILSNVIPYLETISNLPSLWRQDQYDCALWMMTFSSSIFLGLDIGLIISVVSAFFITTVRSHRAKILLLGQIPNTNIYRSINDYREIITIPGVKIFQCCSSITFVNVYYLKHKLLKEVDMVKVPLKEEEIFSLFNSSDTNLQGGKICRCFCNCDDLEPLPRILYTERFENKLDPEASSINLIHCSHFESMNTSQTASEDQVPYTVSSVSQKNQGQQYEEVEEVWLPNNSSRNSSPGLPDVAESQGRRSLIPYSDASLLPSVHTIILDFSMVHYVDSRGLVVLRQICNAFQNANILILIAGCHSSIVRAFERNDFFDAGITKTQLFLSVHDAVLFALSRKVIGSSELSIDESETVIRETYSETDKNDNSRYKMSSSFLGSQKNVSPGFIKIQQPVEEESELDLELESEQEAGLGLDLDLDRELEPEMEPKAETETKTQTEMEPQPETEPEMEPNPKSRPRAHTFPQQRYWPMYHPSMASTQSQTQTRTWSVERRRHPMDSYSPEGNSNEDV

SEQ ID NO: 11 - protein, 179 aa, Homo sapiens [sequence omitted]

SEQ ID NO: 12 - protein, 1662 aa, Homo sapiens
  misc_feature: Incyte ID No 7494181CD1
MPAGPVIWAFLKPMLLGRILYAPYNPVTKAIMEKVGYDSGNVFLPPVIKYTIRMSLKTAQTTRSLRTKIWAPGPHNSPSHNQIYGRAFIYLQDSIERAIIELQTGRNSQEIAVQVQAIPYPCFMKDNFLTSVSYSLPIVLMVAWVVFIAAFVKKLVYEKDLRLHEYMKMMGVNSCSHFFAWLIESVGFLLVTIVILIIILKFGNILPKTNGFILFLYFSDYSFSVIAMSYLISVFFNNTNIAALIGSLIYIIAFFPFIVLVTVENELSYVLKVFMSLLSPTAFSYASQYIARYEEQGIGLQWENMYTSPVQDDTTSFGWLCCLILADSFIYFLIAWYVRNVFPGTYGMAAPWYFPILPSYWKERFGCAEVKPEKSNGLMFTNIMMQNTNPSASPEYMFSSNIEPEPKDLTVGVALHGVTKIYGSKVAVDNLNLNFYEGHITSLLGPNGAGKTTTISMLTGLFGASAGTIFVYGKDIKTDLHTVRKNMGVCMQHDVLFSYLTTKEHLLLYGSIKVPHWTKKQLHEEVKRTLKDTGLYSHRHKRVGTLSGGMKRKLSISIALIGGSRVVILDEPSTGVDPCSRRSIWDVISKNKTARTIILSTHHLDEAEVLSDRIAFLEQGGLRCCGSPFYLKEAFGDGYHLTLTKKKSPNLNANAVCDTMAVTAMIQSHLPEAYLKEDIGGELVYVLPPFSTKVSGAYLSLLRALDNGMGDLNIGCYGISDTTVEEVFLNLTKESQKNSAMSLEHLTQKKIGNSNANGISTPDDLSVSSSNFTDRDDKILTRGERLDGFGLLLKKIMAILIKRFHHTRRNWKGLIAQVILPIVFVTTAMGLGTLRNSSNSYPEIQISPSLYGTSEQTAFYANYHPSTEALVSAMWDFPGIDNMCLNTSDLQCLNKDSLEKWNTSGEPITNFGVCSCSENVQECPKFNYSPPHRRTYSSQVIYNLTGQRVENYLISTANEFVQKRYGGWSFGLPLTKDLRFDITGVPANRTLAKVWYDPEGYHSLPAYLNSLNNFLLRVNMSKYDAARHGIIMYSHPYPGVQDQEQATISSLIDILVALSILMGYSVTTASFVTYVVREHQTKAKQLQHISGIGVTCYWVTNFIYDMVFYLVPVAFSIGIIAIFKLPAFYSENNLGAVSLLLLLFGYATFSWMYLLAGLFHETGMAFITYVCVNLFFGINSIVSLSVVYFLSKEKPNDPTLELISETLKRIFLIFPQFCFGYGLIELSQQQSVLDFLKAYGVEYPNETFEMNKLGAMFVALVSQGTMFFSLRLLINESLIKKLRLFFRKFNSSHVRETIDEDEDVRAERLRVESGAAEFDLVQLYCLTKTYQLIHKKIIAVNNISIGIPAGECFGLLGVNGAGKTTIFKMLTGDIIPSSGNILIRNKTGSLGHVDSRSSLVGYCPQEDALDDLVTVEEHLYFYARVHGIPEKDIKETVHKLLRRLHLMPFKDRATSMCSYGTKRKLSTALALIGKPSILLLDEPSSGMDPKSKRHLWKIISEEVQNKCSVILTSHSMEECEALCTRLAIMVNGKFQCIGSLQHIKSRFGRGFTVKVHLKNNKVTMETLTKFMQLHFPKTYLKDQHLSMLEYHVPVTAGGVANIFDLLETNKTALNITNFLVSQTTLEEVFINFAKDQKSYETADTSSQGSTISVDSQDDQMES

SEQ ID NO: 13 - protein, 588 aa, Homo sapiens
  misc_feature: Incyte ID No 3697053CD1
MPFKAFDTFKEKILKPGKEGVKNAVGDSLGILQKKSMGQLREEDNIELNEEGRPVQTSRPSPPLCDCHCCGLPKRYIIAIMSGLGFCISFGIRCNLGVAIVEMVNNSTVYVDGKPEIQTAQFNWDPETVGLIHGSFFWGYIMTQIPGGFISNKFAANRVFGAAIFLTSTLNMFIPSAARVHYGCVMCVRILQGLVEESINNRTTTAHAAAINTVVNVSGEGAHEGSYAGAVVAMPLAGVLVQYIGWSSVFYIYGMFGIIWYMFWLLQAYECPAAHPTISNEEKTYIETSIGEGANVVSLSKFSTPWKRFFTSLPVYAIIVANFCRSWTFYLLLISQPAYFEEVFGFAISKVGLLSAVPHMVMTIVVPIGGQLADYLRSRQILTTTAVRKIMNCGGFGMEATLLLVVGFSHTKGVAISFLVLAVGFSGFAISGFNVNHLDIAPRYASILMGISNGVGTLSGMVCPLIVGAMTRHKTREEWQNVFLIAALVHYSGVIFYGVFASGEKQEWADPENLSEEKCGIIDQDELAEEIELNHESFASPKKKMSYGATSQNCEVQKKEWKGQRGATLDEEELTSYQNEERNFSTIS

SEQ ID NO: 14 - protein, 257 aa, Homo sapiens
  misc_feature: Incyte ID No 7473203CD1
MATYGQTCMWPVWISSSYVNLGKAARDIFNKGFGLGLVKLDVRTKSRSAVGFSTSGSFNADTGKAFEVLETKYKRSITGNKSGKIKSSCKRDCINLACDVNFDFAGPAIYASAVFGYEGWLAGYQMTTDSAKSKLTRNNCSGYRMGDFELHTNNNNGAEFGGSVYQRVCDNLDTSVNLARTSSANCTFCLATKYQLHFTASMFAKVNNSSLIGVEGKRLHLHSDSEPAVKLALSALLDKKCINGGGQRLGFVLELET

SEQ ID NO: 15 - protein, 473 aa, Homo sapiens
  misc_feature: Incyte ID No 4697002CD1
MALKDTGSGGSTILPISEMVSSSSSPGASAAAAPGPCAPSPFPEVVELNVGGQVYVTKHSTLLSVPDSTLASMFSPSSPRGGARRRGELPRDSRARFFIDRDGFLFRYVLDYLRDKQLALPEHFPEKERLLREAEYFQLTDLVKLLSPKVTKQNSLNDEGCQSDLEDNVSQGSSDALLLRGAAAAVPSGPGAHGGGGGGGAQDKRSGFLTLGYRGSYTTVRDNQADAKFRRVARIMVCGRIALAKEVFGDTLNESRDPDRQPEKYTSRFYLKFTYLEQAFDRLSEAGFHMVACNSSGTAAFVNQYRDDKIWSSYTEYIFFRPPQKIVSPKQEHEDRKHDKVTDKGSESGTSCNELSTSSCDSHSEASTPQDNPSSAQQATAHQPNTLTLDRPSKKAPVQWIPPPDKRRNSELFQTLISKSRETNLSKKKVCEKLSVEEEMKKCIQDFKKIHIPDYFPERKRQWQSELLQKYGL

SEQ ID NO: 16 - protein, 1095 aa, Homo sapiens
  misc_feature: Incyte ID No 5632139CD1
MPLMMSEEGFENEESDYHTLPRARIMQRKRGLEWFVCDGWKFLCTSCCGWLINICRRKKELKARTVWLGCPEKCEEKHPRNSIKNQKYNVFTFIPGVLYEQFKFFLNLYFLVISCSQFVPALKIGYLYTYWAPLGFVLAVTMTREAIDEFRRFQRDKEVNSQLYSKLTVRGKVQVKSSDIQVGDLIIVEKNQRIPSDMVFLRTSEKAGSCFIRTDQLDGETDWKLKVAVSCTQQLPTLGDLVSISANVYAQKPQMDIHSFEGTFTREDSDPPIHESLSIENTLWASTIVASGTVIGVVIYTGKETRSVMNTSNPKNKVGLLDLELNRLTKALFLALVALSIVMVTLQGFVGPWYRNLFRFLLLFSYIIPISLRVNLDMGKAVYGWMMMKDENIPGTVVRTSTIPEELGRLVYLLTDKTGTLTQNEMIFKRLHLGTVSYGADTMDEIQSHVRDSYSQMQSQAGGNNTGSTPLRKAQSSAPKVRKSVSSRIHEAVKAIVLCHNVTPVYESRAGVTEETEFAEADQDFSDENRTYQASSPDEVALVQWTESVGLTLVSRDLTSMQLKTPSGQVLSFCILQLFPFTSESKRMGVIVRDESTAEITFYMKGADVAMSPIVQYNDWLEEECGNMAREGLRTLVVAKKALTEEQYQDFESRYTQAKLSMHDRSLKVAAVVESLEREMELLCLTGVEDQLQADVRPTLEMLRNAGIKIWMLTGDKLETATCIAKSSHLVSRTQDIHIFRQVTSRGEAHLELNAFRRKHDCALVISGDSLEVCLKYYEHEFVELACQCPAVVCCRCSPTQKARIVTLLQQHTGRRTCAIGDGGNDVSMIQAADCGIGIEGKEGKQASLAADFSITQFRHIGRLLMVHGRNSYKRSAALGQFVMHRGLIISTMQAVFSSVFYFASVPLYQGFLMVGYATIYTMFPVFSLVLDQDVKPEMAMLYPELYKDLTKGRSLSFKTFLIWVLISIYQGGILMYGALVLFESEFVHVVAISFTALILTELLMVALTVRTWHWLMVVAEFLSLGCYVSSLAFLNEYFGIGRVSFGAFLDVAFITTVTFLWKVSAITVVSCLPLYVLKYLRRKLSPPSYCKLAS

SEQ ID NO: 17 - protein, 758 aa, Homo sapiens
  misc_feature: Incyte ID No 7506184CD1
MPKPPKPRNNLEDRHNPGIQGRREHRPGPGRVRAASSPGGSAPRAERRLWGEGWESGAAPHPHSSRVSALRPCGVVGAWVGMGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANEGFDPSTLSDPLYVLDQAVGAYLPLTTARYSALLAGNLRI

SEQ ID NO: 18 - DNA, 1929 bp, Homo sapiens
  misc_feature: Incyte ID No 551243CB1
gggcgggagggcagcgcctgaagggcggtggggtggcggggttcctgcgcgcggcccgccatggaggtggaggaggcgttccaggcggtgggggagatgggcatctaccagatgtacttgtgcttcctgctggccgtgctgctgcagctctacgtggccacggaggccatcctcattgcactggttggggccacgccatcctaccactgggacctggcagagctcctgccaaatcagagccacggtaaccagtcagctggtgaagaccaggcctttggggactggctcctgacagccaacggcagtgagatccataagcacgtgcatttcagcagcagcttcacctccatcgcctcggagtggtttttaattgccaacagatcctacaaagtcagtgcagcaagctcttttttcttcagtggtgtatttgttggagttatctcttttggtcagctttcagatcgcttcggaaggaaaaaagtctatctcacaggttttgctcttgacatcttatttgcaattgcaaatggattttccccctcatatgagttctttgcagtaactcgcttcctggtgggcatgatgaatggagggatgtcgctggtggcctttgtcttgcttaatgaatgtgtgggcaccgcctactgggcacttgcaggatcgattggcggcctcttctttgcagttggcattgcccaatatgccctgttaggatacttcatccgctcctggaggaccctagccattctggttaacctgcagggaacggtggtctttctcttatctttattcattcctgaatcacctcgttggttatactcccagggtcgactgagtgaggctgaagaggcgctgtacctcattgccaagaggaaccgcaaactcaagtgcacgttctcactaacacacccagccaacaggagctgcagggagactggaagtttcctggatctctttcgttaccgggtcctgttaggacacactttgatcctgatgttcatctggtttgtgtgcagcttggtgtattatggcctaactctgagtgcgggtgatctaggtggaagtatttatgccaacctggccctgtctggcctcatagagattccatcttaccctctctgtatctacttgattaaccaaaaatggtttggtcggaagcgaacattatcagcatttctgtgcctaggaggactggcttgtcttattgtaatgtttcttccagaaaagaaagacacaggtgtgtttgcagtggtgaacagccattccttgtccttgctggggaagctgaccatcagtgctgcctttaacattgtttatatctacacctctgagctttaccctacagtcatcaggaatgttgggcttggaacttgttccatgttctcccgagttggtgggattattgctcccttcatcccctcactgaaatatgtgcaatggtctttaccattcattgtcttcggagccacgggtctgacctccggcctcctgagtttgttattgccggagacccttaacagtccgctgctagaaacattctccgaccttcaggtgtattcgtatcgcaggctgggagaagaagcattatctttacaggctttggacccccaacagtgtgtggacaaggagagctctttagggagtgagagtgaggaagaggaagaattttatgatgcagatgaagagactcagatgatcaagtgaagagccccagattccccctaagaagcaaaggatcgtcttttatgcctctggctaaggcaggttcttccatgactcctaagagagttgtaaaaatagaggcttgacttgaatgtacatagatggtacctggcatggactgatgtttttaggcacagaagttggagaagagatttcatgaaagacaacatcactgcattgagagaatagttgttaatttgtttagaa

SEQ ID NO: 19 - DNA, 5302 bp, Homo sapiens
  misc_feature: Incyte ID No 7493587CB1
ggccaggcggcgcgctgaccgcggtctccgtgcgtcccgcaggcggggagctcgcaccgccgcgcccgggccgcgagtgatgataacctaagaggccggcgcgggcgggcgtgagcggcggaggagccgggcgcggcgacacgcggccatggagcgggagccggcggggaccgaggagcccgggcctccgggacggcggaggcgccgagagggcaggacgcgcacggtgcgctccaacctgctgccgcccccgggcgccgaggaccctgcggctggcgcggccaagggcgagcggcgacggcggcgcgggtgtgcccagcacctggccgacaaccggctcaagactaccaagtacacgctgctgtccttcctgcccaagaacctgttcgagcagttccaccgcccggccaacgtgtactttgtcttcatcgcgctgctcaacttcgtgccggcggtgaacgccttccagcccggcctggcactggcgccggtgctcttcatcctggccatcacggccttcagggacctgtgggaggactacagccgccaccgctccgaccacaagatcaaccacctgggctgcctggtcttcagcagggaagaaaagaaatacgtgaaccgattctggaaagaaatccacgtgggagactttgtgcgtcttcgctgcaacgaaatcttccctgcggacattctgctgctctcctccagtgaccccgacgggctatgccacatcgagaccgccaacctggatggagagaccaacctgaagcggcggcaggtggtccgcggcttctcggagcttgtctccgaattcaatcctttgacgttcaccagcgtgatcgaatgcgagaagccaaacaacgacctgagtaggtttcgcggctgcatcatacatgacaacgggaaaaaggccgggctgtataaagaaaacctgctgctgaggggctgcacccttaggaacacggacgcagtcgtcggcattgtcatctacgcaggacatgaaaccaaggctctgctgaacaacagtgggccccgctacaagcgcagcaagctggagaggcagatgaactgcgacgtgctctggtgtgtcctgctccttgtttgcatgtctctgttttcagcagtcggacatggactgtggatatggcggtatcaagagaagaagtcattattttatgtccccaagtctgatggaagctccttatccccagtcacagctgcagtttactcatttttaacaatgataatagttctgcaggttttgatcccaatttccttatacgtttccattgaaattgttaaagcatgccaagtgtacttcattaaccaggacatgcagttgtatgacgaagaaacagactcgcagctgcagtgccgagctctgaacatcacggaagacttaggacagatacagtacattttctcagataaaactggcactttgacagagaataagatggttttccgaagatgcactgtgtctggtgtagaatattctcatgatgcaaatgcgcagcgtctggccaggtaccaagaggcagactcggaggaggaggaggtggtgcccagagggggctcggtgtcccagcgcggcagcatcggcagccaccagagtgtccgggtggtgcacagaacccagagcaccaagtcccaccggcgcacgggcagccgggccgaggccaagagggccagcatgctgtccaagcacacggccttcagcagccccatggagaaggatatcacgcccgacccaaagctgctggagaaggtgagtgagtgtgacaagagcctagccgtggcgaggcatcaggagcacctgctggcccacctctcgcctgagctgtctgacgtctttgatttcttcatcgcactcaccatctgcaacacagtcgtcgtcacgtccccggatcagccacgaacaaaggtgagggtgaggtttgagctgaagtccccggtgaagacgatagaagacttcctgcggaggttcacacccagctgcctgacctcaggctgcagcagcatcgggagcctggccgccaacaagtccagccacaagttgggctccagcttcccgtccaccccgtccagcgacggcatgcttctcaggctggaggagaggctgggccagcccacctcggccatcgccagcaacggctacagcagccaggcggacaactgggcctcggagcttgctcaggagcaggagtcagagcgcgagctgcggtacgaggcggagagcccggatgaggccgcactggtgtatgcggccagagcctacaactgcgtgcttgtggagcggctgcacgaccaagtgtcagtggagctgccccacctgggcaggctcaccttcgagctcctgcacacactgggtttcgattccgtccgcaagaggatgtcagtggtgatccggcacccgcttaccgatgagatcaacgtctacaccaagggggccgactcagtggtcatggatctcctgcagccctgctcttcagttgacgccagagggaggcatcaaaaaaagattcggagcaaaactcagaattacctcaacgtgtatgcggcggaaggcctgcgcaccttgtgcatcgccaagagagttctgagtaaagaagagtatgcctgctggttgcaaagccacctagaagccgaatcctccctggaaaacagcgaggagctcctcttccagtctgccattcgcctggagaccaacctgcacttgttaggtgccactgggattgaagaccgcctgcaggacggagtccctgaaactatttctaaattgcgtcaagcgggcctgcagatttgggttctcactggtgacaaacaagaaacagctgtcaacattgcatatgcctgcaaactgctggaccacgacgaggaggtcatcaccctgaatgccacctcccaggaggcgtgtgcagccctgctagaccagtgcctatgctacgtgcagtccagaggcctccagagagcccctgagaagaccaagggcaaagtgagcatgaggttctcctctctctgcccaccctccacgtccactgcctctggccgcagacccagcctcgtgatcgatgggagaagcctggcctacgctctcgagaaaaacctggaggacaaattcctcttccttgccaagcagtgccgctccgtcctctgctgtcggtcgacgcctctgcagaagagcatggtggtgaagctggtgcggagcaagctcaaggccatgaccctggccataggtgatggagccaatgatgtcagcatgatccaggtggcagatgtgggtgtgggaatctccggccaggagggtatgcaggcagtgatggccagcgactttgcagtgccgaaattccgatacctggagaggctcttgattcttcacgggcattggtgctactcccgacttgccaacatggtgctgtacttcttctacaaaaacacaatgttcgtgggcctcctgttttggttccagtttttctgtggcttctctgcatctaccatgattgaccagtggtatctaatcttctttaatctgctcttctcgtcacttcccccgctcgtgactggggtgctggacagggatgtgccagccaatgtgctgctgaccaacccgcagctctacaagagtggccagaacatggaggaataccggccacgaacgttctggtttaacatggctgacgccaccttccagagcctggtttgcttttccattccttacctggcctactatgactcgaacgtggacctgtttacctgggggacccctattgtgacaatcgcgctgctcactttcctgctccacctgggcattgaaaccaaaacctggacctggctcaactggataacgtgtggcttcagtgtccttttgtttttcaccgtggctttgatttacaatgcgtcttgtgccacgtgctatcctccgtccaacccttactggactatgcaagccttactgggtgacccagtgttttacttgacttgcctgatgacgcctgtcgctgcactgctgcccagattgtttttcagatccctccaggggagcgttttccccacacaacttcagctggcacgtcagttgaccaggaagtcccccaggagatgcagtgctcccaaagagacctttgctcagggacgcctcccgaaggactcgggaaccgagcactcatcagggaggacagtcaagacctctgtgcccctgtcccagccttcttggcacacacagcagccggtctgctccctggaggccagcggggagcccagcacagtggacatgagcatgccagtgagggagcacaccctgctggaggggctgagcgcaccggcccccatgtcctctgcgccaggggaggctgtcctgaggagtccaggagggtgtcctgaggagtccaaggtgagagctgccagcaccggcagggtgacccccctgtcttccctcttcagcctgcctaccttcagcttactcaactggatttcctcctggtcgctggtcagcaggctggggagtgtcttacagttctcccggacggagcagcttgcagatggacaagcgggacgtggacttcctgtccagccccactcaggccgatcaggacttcaagggccagaccacagactacttataggagcatcttcaaggcggtcacagtgaaaaccttgaaatggccttttttaatatatataaataaatgttaatattatttatgtttattatttgcacagaagagttctagggagatgtatttctaaatgtttcccaggctaatacaggaaacaagaggtaccaaaaaagaaagtttattttttaaaattctaagtagagtatattgaaaagaaaaagaagagccttaacatatataaaagtttaaagaagagtaacacttgaaaagtgtgtttagatttattttttcatctcatttttaagaacaagcagtacgatttgttttcttcaacatgtgtgactgcgcactgagtacaaatgtgtgactgctcatggttaatgcaggcaggtgtgaacatgggggaacaatgagcagagatggcagagggcagagcacatggcccccagaggcttccagtctcactgacacaggagggctgggctccacttcatccagatgaaggaaaggaagacctcaagaaaaattcacagttgagtgcatcccagcattctgttccgggcaggcatttcaggaagaccgccttgtaggtattacatccctggtgtcgtattttgcctgttaaatcgtaacaagcaataaacaactttcactttgcaaagacaaaaaaaaaaaaaaaaaaagat

SEQ ID NO: 20 - DNA, 2994 bp, Homo sapiens
  misc_feature: Incyte ID No 4505840CB1
gcgatccaaacgccctggctctcaggcctggactctagggcttagccagatgcctaaaccgcccaagccgagaaacaacttagaagacagacataaccctgggattcagggaaggcgcgagcaccgcccaggacctggtagggtgcgagccgcgagcagtccgggagggagcgcgcctagggcggagcgtaggctgtggggggagggctgggagtccggggccgccccacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctacaaaaaaaaaaaaaaaaaaaaaaaaaaaaaaaaaaaaataaaaaaaaaaggggggcccccctaaggggtccccaactttgcctggggggcattggggttaaaaccccttttaagggtcccccaaaatttatttccgggggggttttttaaaaagggggtggggggaaacccccggggtttccccaattttacccctttcacccccccccctgnnccttttggaccnnnnnnnnnnnnnnnnnnnnnnnnnnnnnnnnnnnnnnnnnnnnnnnnnnnnnnnttnnnnnnnnnnnatttctcgcctttggtcacaggttgctgacgaatagagggatgctttctctttatcccgcacc

SEQ ID NO: 21 - DNA, 2094 bp, Homo sapiens
  misc_feature: Incyte ID No 7484873CB1
tgagcccctagctgtgctggtccgggctggcctctctaagacagtgcaggccacgtgatccatcctcctagaggcagtgagcaggtgagggacccctaccacagccaggaggaaaaagctaggcgtccactttccgcagccatgctcaaacagagtgagaggagacggtcctggagctacaggccctggaacacgacggagaatgagggcagccaacaccgcaggagcatttgctccctgggtgcccgttccggctcccaggccagcatccacggctggacagagggcaactataactactacatcgaggaagacgaagacggcgaggaggaggaccagtggaaggacgacctggcagaagaggaccagcaggcaggggaggtcaccaccgccaagcccgagggccccagcgaccctccggccctgctgtccacgctgaatgtgaacgtgggtggccacagctaccagctggactactgcgagctggccggcttccccaagacgcgcctaggtcgcctggccacctccaccagccgcagccgccagctaagcctgtgcgacgactacgaggagcagacagacgaatacttcttcgaccgcgacccggccgtcttccagctggtctacaatttctacctgtccggggtgctgctggtgctcgacgggctgtgtccgcgccgcttcctggaggagctgggctactggggcgtgcggctcaagtacacgccacgctgctgccgcacctgcttcgaggagcggcgcgacgagctgagcgaacggctcaagatccagcacgagctgcgcgcgcaggcgcaggtcgaggaggcggaggaactcttccgcgacatgcgcttctacggcccgcagcggcgccgcctctggaacctcatggagaagccattctcctcggtggccgccaaggccatcggggtggcctccagcaccttcgtgctcgtctccgtggtggcgctggcgctcaacaccgtggaggagatgcagcagcactcggggcagggcgagggcggcccagacctgcggcccatcctggagcacgtggagatgctgtgcatgggcttcttcacgctcgagtacctgctgcgcctagcctccacgcccgacctgaggcgcttcgcgcgcagcgccctcaacctggtggacctggtggccatcctgccgctctaccttcagctgctgctcgagtgcttcacgggcgagggccaccaacgcggccagacggtgggcagcgtgggtaaggtgggtcaggtgttgcgcgtcatgcgcctcatgcgcatcttccgcatcctcaagctggcgcgccactccaccggactgcgtgccttcggcttcacgctgcgccagtgctaccagcaggtgggctgcctgctgctcttcatcgccatgggcatcttcactttctctgcggctgtctactctgtggagcacgatgtgcccagcaccaacttcactaccatcccccactcctggtggtgggccgcggtgagcatctccaccgtgggctacggagacatgtacccagagacccacctgggcaggttttttgccttcctctgcattgcttttgggatcattctcaacgggatgcccatttccatcctctacaacaagttttctgattactacagcaagctgaaggcttatgagtataccaccatacgcagggagaggggagaggtgaacttcatgcagagagccagaaagaagatagctgagtgtttgcttggaagcaacccacagctcaccccaagacaagagaattagtattttataggacatgtggctggtagattccatgaacttcaaggcttcattgctctttttttaatcattatgattggcagcaaaaggaaatgtgaagcagacatacacaaaggccatttcgttcacaaagtactgcctctagaaatactcattttggcccaaactcagaatgtctcatagttgctctgtgttgtgtgaaacatctgaccttctcaatgacgttgatattgaaaacctgaggggagcaacagcttagatttttcttgtagcttctcgtggcatctagctcaataaatatttttggacttgtaaaaa

SEQ ID NO: 22 - DNA, 5846 bp, Homo sapiens
  misc_feature: Incyte ID No 3559054CB1
gcttggtctggctacaccccttttcagaaaccaggctgtgtaagagctgctggagtaggcacccatttaaagaaaaaatgaagaagcagcaataaagaagttgtaatcgttacctagacaaacagagaactggttttgacagtgtttctagagtgctttttattattttcctgacagttgtgttccaccatgattactttctccttcagcgaataggctaaatgaatatgaaacagaaaagcgtgtatcagcaaaccaaagcacttctgtgcaagaattttcttaagaaatggaggatgaaaagagagagcttattggaatggggcctctcaatacttctaggactgtgtattgctctgttttccagttccatgagaaatgtccagtttcctggaatggctcctcagaatctgggaagggtagataaatttaatagctcttctttaatggttgtgtatacaccaatatctaatttaacccagcagataatgaataaaacagcacttgctcctcttttgaaaggaacaagtgtcattggggcaccaaataaaacacacatggacgaaatacttctggaaaatttaccatatgctatgggaatcatctttaatgaaactttctcttataagttaatatttttccagggatataacagtccactttggaaagaagatttctcagctcattgctgggatggatatggtgagttttcatgtacattgaccaaatactggaatagaggatttgtggctttacaaacagctattaatactgccattatagaaatcacaaccaatcaccctgtgatggaggagttgatgtcagttactgctataactatgaagacattacctttcataactaaaaatcttcttcacaatgagatgtttattttattcttcttgcttcatttctccccacttgtatattttatatcactcaatgtaacaaaagagagaaaaaagtctaagaatttgatgaaaatgatgggtctccaagattcagcattctggctctcctggggtctaatctatgctggcttcatctttattatttccatattcgttacaattatcataacattcacccaaattatagtcatgactggcttcatggtcatatttatactcttttttttatatggcttatctttggtagctttggtgttcctgatgagtgtgctgttaaagaaagctgtcctcaccaatttggttgtgtttctccttaccctcttttggggatgtctgggattcactgtattttatgaacaacttccttcatctctggagtggattttgaatatttgtagcccttttgcctttactactggaatgattcagattatcaaactggattataacttgaatggtgtaatttttcctgacccttcaggagactcatatacaatgatagcaactttttctatgttgcttttggatggtctcatctacttgctattggcattatactttgacaaaattttaccctatggagatgagcgccattattctcctttatttttcttgaattcatcatcttgtttccaacaccaaaggactaatgctaaggttattgagaaagaaatcgatgctgagcatccctctgatgattattttgaaccagtagctcctgaattccaaggaaaagaagccatcagaatcagaaatgttaagaaggaatataaaggaaaatctggaaaagtggaagcattgaaaggcttgctctttgacatatatgaaggtcaaatcacggcaatcctgggtcacagtggagctggcaaatcttcactgctaaatattcttaatggattgtctgttccaacagaaggatcagttaccatctataataaaaatctctctgaaatgcaagacttggaggaaatcagaaagataactggcgtctgtcctcaattcaatgttcaatttgacatactcaccgtgaaggaaaacctcagcctgtttgctaaaataaaagggattcatctaaaggaagtggaacaagagattttgcttttagatgaaccaactactggattggatcccttttccagagatcaagtgtggagcctcctgagagagcgtagagcagatcatgtgatccttttcagtacccagtccatggatgaggctgacatcctggctgatagaaaagtgatcatgtccaatgggagactgaagtgtgcaggttcttctatgtttttgaaaagaaggtggggtcttggatatcacctaagtttacataggaatgaaatatgtaacccagaacaaataacatccttcattactcatcacatccccgatgctaaattaaaaacagaaaacaaagaaaagcttgtatatactttgccactggaaaggacaaatacatttccagatcttttcagtgatctggataagtgttctgaccagggagtgacaggttatgacatttccatgtcaactctaaatgaagtctttatgaaactggaaggacagtcaactatcgaacaaggtaaagccatttgtataaatttcgaacaagtggagatgataagagactcagaaagcctcaatgaaatggagctggctcactcttccttctctgaaatgcagacagctgtgagtgacatgggcctctggagaatgcaagtctttgccatggcacggctccgtttcttaaagttaaaacgtcaaactaaagtgttattgaccctattattggtatttggaatcgcaatattccctttgattgttgaaaatataatatatgctatgttaaatgaaaagatcgattgggaatttaaaaacgaattgtattttctctctcctggacaacttccccaggaaccccgtaccagcctgttgatcatcaataacacagaatcaaatattgaagattttataaaatcactgaagcatcaaaatatacttttggaagtagatgactttgaaaacagaaatggtactgatggcctctcatacaatggagctatcatagtttctggtaaacaaaaggattatagattttcagttgtgtgtaataccaagagattgcactgttttccaattcttatgaatattatcagcaatgggctacttcaaatgtttaatcacacacaacatattcgaattgagtcaagcccatttcctcttagccacataggactctggactgggttgccggatggttcctttttcttatttttggttctatgtagcatttctccttatatcaccatgggcagcatcagtgattacaagaaaaatgctaagtcccagctatggatttcaggcctctacacttctgcttactggtgtgggcaggcactagtggacgtcagcttcttcattttaattctccttttaatgtatttaattttctacatagaaaacatgcagtaccttcttattacaagccaaattgtgtttgctttggttatagttactcctggttatgcagcttctcttgtcttcttcatatatatgatatcatttatttttcgcaaaaggagaaaaaacagtggcctttggtcattttacttcttttttgcctccaccatcatgttttccatcactttaatcaatcattttgacctaagtatattgattaccaccatggtattggttccttcatataccttgcttggatttaaaacttttttggaagtgagagaccaggagcactacagagaatttccagaggcaaattttgaattgagtgccactgattttctagtctgcttcataccctactttcagactttgctattcgtttttgttctaagatacatggaactaaaatgtggaaagaaaagaatgcgaaaagatcctgttttcagaatttccccccaaagtagagatgctaagccaaatccagaagaacccatagatgaagatgaagatattcaaacagaaagaataagaacagtcactgctctgaccacttcaatcttagatgagaaacctgttataattgccagctgtctacacaaagaatatgcaggccagaagaaaagttgcttttcaaagaggaagaagaaaatagcagcaagaaatatctctttctgtgttcaagaaggtgagattttgggattgctaggacccagtggtgctggaaaaagttcatctattagaatgatatctgggatcacaaagccaactgctggagaggtggaactgaaaggctgcagttcagttttgggccacctggggtactgccctcaagagaacgtgctgtggcccatgctgacgttgagggaacacctggaggtgtatgctgccgtcaaggggctcagggaagcggacgcgaggctcgccatcgcaagattagtgagtgctttcaaactgcatgagcagctgaatgttcctgtgcagaaattaacagcaggaatcacgagaaagttgtgttttgtgctgagcctcctgggaaactcacctgtcttgctcctggatgaaccatctacgggcatagaccccacagggcagcagcaaatgtggcaggcaatccaggcagtcgttaaaaacacagagagaggtgtcctcctgaccacccataacctggctgaggcggaagccttgtgtgaccgtgtggccatcatggtgtctggaaggcttagatgcattggctccatccaacacctgaaaaacaaacttggcaaggattacattctagagctaaaagtgaaggaaacgtctcaagtgactttggtccacactgagattctgaagcttttcccacaggctgcagggcaggaaaggtattcctctttgttaacctataagctgcccgtggcagacgtttaccctctatcacagacctttcacaaattagaagcagtgaagcataactttaacctggaagaatacagcctttctcagtgcacactggagaaggtattcttagagctttctaaagaacaggaagtaggaaattttgatgaagaaattgatacaacaatgagatggaaactcctccctcattcagatgaaccttaaaacctcaaacctagtaattttttgttgatctcctataaacttatgttttatgtaataattaatagtatgtttaattttaaagatcatttaaaattaacatcaggtatattttgtaaatttagttaacaaatacataaattttaaaattattcttcctctcaaacataggggtgatagcaaacctgtgataaaggcaatacaaaatattagtaaagtcacccaaagagtcaggcactgggtattgtggaaataaaactatataaacttagaattttttaaaaatatgacttttttaccttttacaaaacattctcttgctgaaatatgtgaagggtatattcagtagccaagagttgcatgactacttcacaccagttcatgatacaacaggtatacaggttttcttttataaccaactacaactcaagagtcttctgaaagtgttccagaaattgctttaaaactcaaaagtaaggggccaggtgcagtggctcacgcctgtaatcccagcactttgggaggccgaggcaggtggatcacaaggtcaggagttcgagactagcctggccaatatggtgaaactccatctctaataaaaatacaaaaattagccgggcgttggcatttgcctgtagtcctagctattcgggaggctgagggaggagaattgcttgaacccgggaggcagaggttgcagtgagccatgtgctagtgcactccagcctgggtgacagagtgagactctgtcaaaaaaaaaaaaaacaaaaaaaaaaacaaaaaaccttcaaggttttggaggtctttggccacaatttgagagccccttttggaaaggtttcccttttacttttgaataaagggtccggatttggc

SEQ ID NO: 23 - DNA, 6813 bp, Homo sapiens
  misc_feature: Incyte ID No 7477526CB1
gtcagagcggaaacctagcgggggtcgggggttcagctcggggcggtgggaaacaccccgggagaggaggcagctctgtgattccgctccgggccggagggagaggagttcggaggtggcttgagctgagaatccggaggagagaaggcgcattttgagctgccacgggcacagatctcagacccggacgctgactagccgggggtcgcggctttccgaggcggctggagaagtgcccagacggccgtccctccgcgcccctgcgcgtccccgtgcgcccagtgttccccgtgcaggagtcccggagcgatgatagcgccggttacgtcccagaaatcctggattaaaggagtatttgacaagagagaatgtagcacaatcatacccagctcaaaaaatcctcacaggtgttactgtggccgactgattggagaccatgctgggatagattattcctggaccatctcagctgccaagggtaaagaaagtgaacaatggtctgttgaaaagcacacaacgaaaagcccaacagatacttttggcacgattaatttccaagatggagagcacacccatcatgccaagtatattagaacttcttatgatacaaaactggatcatctgttacatttaatgttgaaagagtggaaaatggaactgcccaagcttgtgatctcagtccatgggggcatccagaactttactatgccctctaaatttaaagagattttcagccaaggtttggttaaagctgcagagacaacaggagcgtggataataactgaaggcatcaatacaggagtgtccaagcatgttggggatgccttgaaatcccattcctctcattccttgagaaaaatctggacagttggaatccctccttggggtgtcattgagaaccagagagaccttattggaaaagatgtggtgtgcctgtaccagactctggataaccccctcagcaagctcacaacactcaacagcatgcactcgcacttcatcctgtctgatgatgggaccgtgggcaagtatggaaatgaaatgaagctcagaaggaacctggagaagtacctctctctgcagaaaatacactgccgctcaagacaaggcgtgccggtcgtggggctggtggtggaaggcggtcccaacgtcatcctgtcagtgtgggagactgtcaaggacaaggacccagtggtggtgtgtgagggcacaggtagggcggctgacctcctggccttcacacacaaacacctggcagatgaagggatgctgcgacctcaggtgaaagaggagatcatctgcatgattcagaacactttcaactttagtcttaaacagtccaagcaccttttccaaattctaatggagtgtatggttcacagggattgtattaccatatttgatgctgactctgaagagcagcaagacctggacttagcaatcctaacagctttgctgaagggcacaaatttatcagcgtcagagcaattaaatctggcaatggcttgggacagggtggacattgccaagaaacatatcctaatttatgaacaacactggaagcctgatgccctggaacaagcaatgtcagatgctttagtgatggatcgggtggattttgtgaagctcttaatagaatatggagtgaacctccatcgctttcttaccatccctcgactggaagagctctacaatacaaaacaaggacctactaatacactcttgcatcatctcgtccaagatgtgaaacagcatacccttctttcaggctaccgaataaccttgattgacattggattagtagtagaatacctcattggtagagcatatcgcagcaactacactagaaaacatttcagagccctctacaacaacctctacagaaaatacaagcaccagagacactcctcaggaaatagaaatgagtctgcagaaagtacgctgcactcccagttcattagaactgcacagccatacaaattcaaggaaaagtctatagtccttcataaatcaaggaagaagtcaaaagaacaaaatgtatcagatgaccctgagtctactggctttctttacccttacaatgacctgctggtttgggctgtgctgatgaaaaggcagaagatggctatgttcttctggcagcatggagaggaggccacggttaaagccgtgattgcgtgtatcctctaccgggcaatggcccatgaagctaaggagagtcacatggtggatgatgcctcagaagagttgaagaattactcaaaacagtttggccagctggctctggacttgttggagaaggcattcaagcagaatgagcgcatggccatgacgctgttgacgtatgaactcaggaactggagcaattcgacctgcctgaaactggccgtgtcgggaggattacgaccctttgtttcacatacttgtacccagatgctactgacagacatgtggatggggaggctgaaaatgaggaaaaactcttggttaaagattattataagcattattttaccacccaccattttgacactggaatttaaaagcaaagctgagatgtcacatgttccccagtcccaggacttccaatttatgtggtattacagtgaccagaacgccagcagttccaaagaaagtgcttctgtgaaagagtatgatttggaaaggggccatgatgagaaactggatgaaaatcagcattttggtttggaaagtgggcaccaacaccttccgtggaccaggaaagtctatgagttctacagtgctccaattgtcaagttttggttttatacgatggcgtatttggcattcctcatgctgttcacttacaccgtgttggtggagatgcagccccagcccagcgtgcaggagtggcttgttagcatttacatcttcaccaatgctattgaggtggtcagggagatctgtatttcagaacctgggaagtttacccaaaaggtgaaggtatggattagtgagtactggaacttaacagaaactgtggccattggcctgttttcagctggcttcgtccttcgatggggtgaccctccttttcacacagcgggaagactgatctactgcatagacatcatattctggttctcacggctcctggacttctttgctgtgaatcaacatgcaggtccatatgtgaccatgattgcaaaaatgacagcaaacatgttctatattgtgatcatcatggccatagtcctgctgagctttggagtggcacgcaaggccatcctttcgccaaaagagccaccatcttggagtctagctcgagatattgtatttgagccatactggatgatatacggagaagtctatgctggagaaatagatgtttgttcaagccagccatcctgccctcctggttcttttcttactccattcttgcaagctgtctacctcttcgtgcaatatatcatcatggtgaacctgttgattgctttcttcaacaacgtttacttagatatggaatccatttcaaataacctgtggaaatacaaccgctatcgctacatcatgacctaccacgagaagccctggctgcccccacctctcatcctgctgagccacgtgggccttctcctccgccgcctgtgctgtcatcgagctcctcacgaccaagaagagggtgacgttggattaaaactctacctcagtaaggaggatctgaaaaaacttcatgattttgaggagcagtgcgtggaaaaatacttccatgagaagatggaagatgtgaattgtagttgtgaggaacgaatccgagtgacatcagaaagggttacagagatgtacttccagctgaaagaaatgaatgaaaaggtgtcttttataaaggactccttactgtctttggacagccaggtgggacacctgcaggatctctctgccctgactgtggataccctgaaagtcctttctgctgttgacactttgcaagaggatgaggctctcctggccaagagaaagcattctacttgcaaaaaacttccccacagctggagcaatgtcatctgtgcagaggttctaggcagcatggagatcgctggagagaagaaataccagtattatagcatgccctcttctttgctgaggagcctggctggaggccggcatcccccaagagtgcagaggggggcacttcttgagattacaaacagtaaaagagaggctacaaatgtaagaaatgaccaggaaaggcaagaaacacaaagtagtatagtggtttctggggtgtctcctaacaggcaagcacactcaaagtatggccagtttcttctggtcccctctaatctaaagcgagttcctttttcagcagaaactgtcttgcctctgtccagaccctctgtgccagatgtgctggcaactgaacaggacatccagactgaggttcttgttcatctgactgggcagaccccagttgtctctgactgggcatcagtggatgaacccaaggaaaagcacgagcctattgctcacttactggatggacaagacaaggcagagcaagtgctacccactttgagttgcacacctgaacccatgacaatgagctcccctctttcccaagccaagatcatgcaaactggaggtggatatgtaaactgggcattttcagaaggtgatgaaactggtgtgtttagcatcaagaaaaagtggcaaacctgcttgccctccacttgtgacagtgattcctctcggagtgaacagcaccagaagcaggcccaggacagctccctatctgataactcaacaagatcggcccagagtagtgaatgctcagaggtgggaccatggcttcagccaaacacatccttttggatcaatcctctccgcagatacaggcccttcgctaggagtcatagttttagattccataaggaggagaaattgatgaagatctgtaagattaaaaatctttcaggctcttcagaaatagggcagggagcatgggtcaaagcgaaaatgctaaccaaagacaggagactgtcaaagaaaaagaagaatactcaaggactccaggtgccaatcataacagtcaatgcctgctctcagagtgaccagttgaatccagagccaggagaaaacagcatctctgaagaggagtacagcaagaactggttcacagtgtccaaatttagtcacacaggtgtagaaccttacatacatcagaaaatgaaaactaaagaaattggacaatgtgctatacaaatcagtgattacctaaagcagtctcaagaggatctcagcaaaaactctttgtggaattccaggagcaccaacctcaataggaactccctgctgaaaagttcaattggagttgacaagatctcagcctccttaaaaagccctcaagagcctcaccatcattattcagccattgaaaggaataatttaatgaggctttctcagaccataccatttacaccagtccaactgtttgcaggagaagaaataactgtctacaggttggaggagagttcccctttaaaccttgataaaagcatgtcctcttggtctcagcgtgggagagcggcaatgatccaggtattgtcccgagaggagatggatgggggcctccgtaaagctatgagagtcgtcagcacttggtctgaggatgacattctcaagccgggacaagttttcattgtcaagtcctttcttcctgaggttgtgcggacatggcataaaatcttccaggagagcactgtgcttcatctttgcctcagggaaattcaacaacaaagagctgctcaaaaattgatctataccttcaaccaagtgaaaccacaaaccataccctacacaccaaggttcctggaagttttcttaatctactgccattcagccaaccagtggttgaccattgagaagtatatgacaggggagttccggaagtataacaacaacaatggtgatgaaatcacccccaccaacaccctggaggagctgatgttggctttctctcactggacctatgagtacactcggggagagctgctggttttagatttgcaaggtgttggagaaaatttgacagatccatctgttataaaacctgaagtcaaacaatcaagaggaatggtgtttggaccggccaatttgggggaagatgcaattagaaacttcattgcaaaacatcattgtaactcctgctgccggaagctcaaactcccggatttaaaaagaaatgactattcccctgaaaggataaattccacctttggacttgagataaaaatagaatcagctgaggagcctccagcaagggagacgggtagaaattccccagaagatgatatgcaactataaaaagggaggagcaagaagatcccagtgcttgccctgcctgccaggaactctgtgataacatagattgatcaacgtgatgttgattacatcagcgtctccttgggacacgccttctgagcctcacatctccttctgttcaaaggcctcattggtatatgatcaatgggttctcctagacactgacctctgtccagggcactttgcagctccatcctcaagttccacacgaagatgcttggatgagtcagctgggaatattgttcttgtgtacctcattgctttagctggtcacttggaactttggagcagaatcctgcacattaaaggatggggttgggggggatacatttattttattttctcactatgtatgcagactggaccccctactactatttgtcacctcacccacagattgtatttatgtctatatatatgttcataaaaagttatgtgatttcctcctctgtcttttccacaacataggac

SEQ ID NO: 24 - DNA, 951 bp, Homo sapiens
  misc_feature: Incyte ID No 7487253CB1
ccagccgctcgctcggctccgctccctggctcggctccctgcctccgcgtcgcagcccccgccgtagccgcctccgagcccgccgccacatcctctgagcagaagatggctgtgccacccacgtatgccgatcttggcaaatctgccagggatgtcttcaccaagggctatggatttggcttaataaagcttgatttgaaaacaaaatctgagaatggattggaatttacaagctcaggctcagccaacactgagaccaccaaagtgacgggcagtctggaaaccaagtacagatggactgagtacggcctgacgtttacagagaaatggaataccgacaatacactaggcaccgagattactgtggaagatcagcttgcacgtggactgaagctgaccttcgattcatccttctcacctaacactgggaaaaaaaatgctaaaatcaagacaggttacaagcaggagcacatcaacctgagctgtgacatgcattttgaaattgctgagccttcaatcagaggctttctggtgctaggttacgagggctggctggccggctaccagatgaattttgagactgcaaagtcccaagggacccagagcaactttgcagttggctacaagactgatgaattccagcttcacactaatgtgaatgacgggacagagtttggtggctccatttaccagaaagtgaacaagaagttggagagcactgtgaatcttggctggacagcagaaaaatgtaaaacttgctttgaaatagcagccaagtatcagatcaaccctgatgcttgctttttggataaactgaacaacttcagcctgttaggtttaggatatattcagaccctaaagccaggtatcagactgacactgtcagctttcctgtatggtaagaacgttcaggctcacaagcttgatctaagactggaatttcaagtgtaa

SEQ ID NO: 25 - DNA, 925 bp, Homo sapiens
  misc_feature: Incyte ID No 2131556CB1
tgggcagcttcatctgcccgcctaggtggctccacggggcgggcccctcggccagggagggcggggcgcacagggagacttaaagagctccccaggtccccacccgcgcctgaccgcggcagctcccaccatggcggagaccaagctccagctgtttgtcaaggcgagtgaggacggggagagcgtgggtcactgcccctcctgccagcggctcttcatggtcctgctcctcaagggcgtacctttcaccctcaccacggtggacacgcgcaggtccccggacgtgctgaaggacttcgcccccggctcgcagctgcccatcctgctctatgacagcgacgccaagacagacacgctgcagatcgaggactttctggaggagacgctggggccgcccgacttccccagcctggcgcctcgttacagggagtccaacaccgccggcaacgacgttttccacaagttctccgcgttcatcaagaacccggtgcccgcgcaggacgaagccctgtaccagcagctgctgcgcgccctcgccaggctggacagctacctgcgcgcgcccctggagcacgagctggcgggggagccgcagctgcgcgagtcccgccgccgcttcctggacggcgacaggctcacgctggccgactgcagcctcctgcccaagctgcacatcgtcgacacggtgtgcgcgcacttccgccaggcgcccatccccgcggagctgcgcggcgtacgccgctacctggacagcgcgatgcaggagaaagagttcaaatacacgtgtccgcacagcgccgagatcctggcggcctaccggcccgccgtgcacccccgctagcgccccaccccgcgtctgtcgcccaataaaggcatctttgtcgggataaaaaaaaaaaaaaaaaaaaaaaaaaaaaaaaaaaaa

SEQ ID NO: 26 - DNA, 7355 bp, Homo sapiens
  misc_feature: Incyte ID No 3254315CB1
tcggcctcgagggtgtgacaacggtcaataatgaaggtggctgcggcgcggcggcaggctcagctgcgccgggcgggggcggcgctggggccgcgcctgtaggactcggggccgacgccgcgggatggggacgcggcgcggggagtgaggcagtggcggcggcggcggtaagcggaacttcggcccgaggggctcgcccgctcccgcctctgtcttgtcggcctccacctgcagccccgcggcccccgcgccccgcgggacccggacggcgacgacgggggaatgtggcgctggatccggcagcagctgggttttgacccaccacatcagagtgacacaagaaccatctacgtagccaacaggtttcctcagaatggcctttacacacctcagaaatttatagataacaggatcatttcatctaagtacactgtgtggaattttgttccaaaaaatttatttgaacagttcagaagagtggcaaacttttattttcttattatatttttggttcagcttatgattgatacacctaccagtccagttaccagtggacttccattattctttgtgataacagtaactgccataaagcagggatatgaagattggttacggcataactcagataatgaagtaaatggagctcctgtttatgttgttcgaagtggtggccttgtaaaaactagatcaaaaaacattcgggtgggtgatattgttcgaatagccaaagatgaaatttttcctgcagacttggtgcttctgtcctcagatcgactggatggttcctgtcacgttacaactgctagtttggacggagaaactaacctgaagacacatgtggcagttccagaaacagcattattacaaacagttgccaatttggacactctagtagctgtaatagaatgccagcaaccagaagcagacttatacagattcatgggacgaatgatcataacccaacaaatggaagaaattgtaagacctctggggccggagagtctcctgcttcgtggagccagattaaaaaacacaaaagaaatttttggtgttgcggtatacactggaatggaaactaagatggcattaaattacaagagcaaatcacagaaacgatctgcagtagaaaagtcaatgaatacatttttgataatttatctagtaattcttatatctgaagctgtcatcagcactatcttgaagtatacatggcaagctgaagaaaaatgggatgaaccttggtataaccaaaaaacagaacatcaaagaaatagcagtaagattctgagatttatttcagacttccttgcttttttggttctctacaatttcatcattccaatttcattatatgtgacagtcgaaatgcagaaatttcttggatcattttttattggctgggatcttgatctgtatcatgaagaatcagatcagaaagctcaagtcaatacttccgatctgaatgaagagcttggacaggtagagtacgtgtttacagataaaactggtacactgacagaaaatgagatgcagtttcgggaatgttcaattaatggcatgaaataccaagaaattaatggtagacttgtacccgaaggaccaacaccagactcttcagaaggaaacttatcttatcttagtagtttatcccatcttaacaacttatcccatcttacaaccagttcctctttcagaaccagtcctgaaaatgaaactgaactaattaaagaacatgatctcttctttaaagcagtcagtctctgtcacactgtacagattagcaatgttcaaactgactgcactggtgatggtccctggcaatccaacctggcaccatcgcagttggagtactatgcatcttcaccagatgaaaaggctctagtagaagctgctgcaaggattggtattgtgtttattggcaattctgaagaaactatggaggttaaaactcttggaaaactggaacggtacaaactgcttcatattctggaatttgattcagatcgtaggagaatgagtgtaattgttcaggcaccttcaggtgagaagttattatttgctaaaggagctgagtcatcaattctccctaaatgtataggtggagaaatagaaaaaaccagaattcatgtagatgaatttgctttgaaagggctaagaactctgtgtatagcatatagaaaatttacatcaaaagagtatgaggaaatagataaacgcatatttgaagccaggactgccttgcagcagcgggaagagaaattggcagctgttttccagttcatagagaaagacctgatattacttggagccacagcagtagaagacagactacaagataaagttcgagaaactattgaagcattgagaatggctggtatcaaagtatgggtacttactggggataaacatgaaacagctgttagtgtgagtttatcatgtggccattttcatagaaccatgaacatccttgaacttataaaccagaaatcagacagcgagtgtgctgaacaattgaggcagcttgccagaagaattacagaggatcatgtgattcagcatgggctggtagtggatgggaccagcctatctcttgcactcagggagcatgaaaaactatttatggaagtttgcagaaattgttcagctgtattatgctgtcgtatggctccactgcagaaagcaaaagtaataagactaataaaaatatcacctgagaaacctataacattggctgttggtgatggtgctaatgacgtaagcatgatacaagaagcccatgttggcataggaatcatgggtaaagaaggaagacaggctgcaagaaacagtgactatgcaatagccagatttaagttcctctccaaattgctttttgttcatggtcatttttattatattagaatagctacccttgtacagtattttttttataagaatgtgtgctttatcacaccccagtttttatatcagttctactgtttgttttctcagcaaacattgtatgacagcgtgtacctgactttatacaatatttgttttacttccctacctattctgatatatagtcttttggaacagcatgtagaccctcatgtgttacaaaataagcccaccctttatcgagacattagtaaaaaccgcctcttaagtattaaaacatttctttattggaccatcctgggcttcagtcatgcctttattttcttttttggatcctatttactaatagggaaagatacatctctgcttggaaatggccagatgtttggaaactggacatttggcactttggtcttcacagtcatggttattacagtcacagtaaagatggctctggaaactcatttttggacttggatcaaccatctcgttacctggggatctattatattttattttgtattttccttgttttatggagggattctctggccatttttgggctcccagaatatgtattttgtgtttattcagctcctgtcaagtggttctgcttggtttgccataatcctcatggttgttacatgtctatttcttgatatcataaagaaggtctttgaccgacacctccaccctacaagtactgaaaaggcacagcttactgaaacaaatgcaggtatcaagtgcttggactccatgtgctgtttcccggaaggagaagcagcgtgtgcatctgttggaagaatgctggaacgagttataggaagatgtagtccaacccacatcagcagatcatggagtgcatcggatcctttctataccaacgacaggagcatcttgactctctccacaatggactcatctacttgttaaaggggcagtagtactttgtgggagccagttcacctcctttcctaaaattcagtgtgatcaccctgttaatggccacactagctctgaaattaatttccaaaatctttgtagtagttcatacccactcagagttataatggcaaacaaacagaaagcattagtacaagcccctcccaacacccttaatttgaatctgaacatgttaaaatttgagaataaagagacatttttcatctctttgtctggtttgtcccttgtgcttatgggactcctaatggcatttcagtctgttgctgaggccattatattttaatataaatgtagaaaaaagagagaaatcttagtaaagagtattttttagtattagcttgattattgactcttctatttaaatctgcttctgtaaattatgctgaaagtttgccttgagaactctatttttttattagagttatatttaaagcttttcatgggaaaagttaatgtgaatactgaggaattttggtccctcagtgacctgtgttgttaattcattaatgcattctgagttcacagagcaaattaggagaatcatttccaaccattatttactgcagtatggggagtaaatttataccaattcctctaactgtactgtaacacagcctgtaaagttagccatataaatgcaagggtatatcatatatacaaatcaggaatcaggtccgttcaccgaacttcaaattgatgtttactaatatttttgtgacagagtataaagaccctatagtgggtaaattagatactattagcatattattaatttaatgtctttatcattggatcttttgcatgctttaatctggttaacatatttaaatttgctttttttctctttacctgaaggctctgtgtatagtatttcatgacatcgttgtacagtttaactatatcaataaaaagtttggacagtatttaaatattgcaaatatgtttaattatacaaatcagaatagtatgggtaattaaatgaatacaaaaagaagagcctctttctgcagccgacttagacatgctcttccctttctataagctagattttagaataaagggtttcagttaataatcttattttcaggttatgtcatctaacttatagcaaactaccacaatacagtgagttctgccagtgtcccagtacaaggcatatttcaggtgtggctgtggaatgtaaaaatgctcaacttgtatcaggtaatgttagcaataaattaaatgctaagaatgattaatcgggtacatgttactgtaattaactcattgcacttcaaaacctaacttccatcctgaatttatcaagtagttcagtattgtcatttgtttttgttttattgaaaagtaatgttgtcttaagatttagaagtgattattagcttgagaactattacccagctctaagcaaataatgattgtatacatattaagataatggttaaatgcggttttaccaagttttcccttgaaaatgtaattcctttatggagatttattgtgcagccctaagcttccttcccatttcatgaatataaggcttctagaattggactggcaggggaaagaatggtagagacagaaattaagactttatccttgtttgcttgtaaactattattttcttgctaatgtaacatttgtctgttccagtgatgtaaggatattaagttattaagctaaatattaattttcaaaaatagtccttctttaacttagatatttcatagctggatttaggaagatctgttattctggaagtactaaaaagaataatacaacgtacaatgtctgcattcactaattcatgttccagaagaggaaataatgaagatatactcagtagagtactaggtgggaggatatggaaatttgctcataaaatctcttataaaacgtgcatataacaaaatgacacccagtaggcctgcattacatttacatgaccgtgtttatttgccatcaaataaactgagtactgacaccagacaaagactccaaagtcataaaatagcctatgaccaactgcagcaagacaggaggtcagctcgcctataatggtgcttaaagtgtgattgatgtaattttctgtactcaccatttgaagttagttaaggagaactttatttttttaaaaaaagtaaatggcaaccactagtgtgctcatcctgaactgttactccaaatccactccgtttttaaagcaaaattatcttgtgattttaagaaaagagttttctatttatttaagaaagtaacaatgcagtctgcaagctttcagtagttttctagtgctatattcatcctgtaaaactcttactacgtaaccagtaatcacaaggaaagtgtcccctttgcatatttctttaaaattctttctttggaaagtatgatgttgataattaacttacccttatctgccaaaaccagagcaaaatgctaaatacgttattgctaatcagtggtctcaaatcgatttgcctccctttgcctcgtctgagggctgtaagcctgaagatagtggcaagcaccaagtcagtttccaaaattgcccctcagctgctttaagtgactcagcaccctgcctcagcttcagcaggcctaggctcaccctgggcggagcaaagtatgggccagggagaactacagctacgaagacctgctgtcgagttgagaaaaggggagaatttatggtctgaattttctaactgtcctctttcttgggtctaaagctcataatacacaaaggcttccagacctgagccacacccaggccctatcctgaacaggagactaaacagaggcaaatcaaccctaggaaatacttgcattctgccctacggttagtaccaggactgaggtcatttctactggaaaagattgtgagattgaacttatctgatcgcttgagactcctaataggcaggagtcaaggccactagaaaattgacagttaagagccaaaagtttttaaaatatgctactctgaaaaatctcgtgaaggctgtaggaaaagggagaatcttccatgttggtgtttttcctgtaaagatcagtttggggtatgatataagcaggtattaataaaaataacacaccaaagagttacgtaaaacatgttttattaattttggtccccacgtacagacattttatttctattttgaaatgagttatctattttcataaaagtaaaacactattaaagtgctgttttatgtgaaataacttgaatgttgttcctataaaaaatagatcataactcatgatatgtttgtaatcatggtaatttagatttttatgaggaatgagtatctggaaatattgtagcaatacttggtttaaaattttggacctgagacactgtggctgtctaatgtaatcctttaaaaattctctgcattgtcagtaaatgtagtatattattgtacagctactcataattttttaaagtttatgaagttatatttatcaaataaaaactttcctatataattaaaaaaaaaaaa

SEQ ID NO: 27 - DNA, 3369 bp, Homo sapiens
  misc_feature: Incyte ID No 7472707CB1
ccgccagccaggcgagagccgtgtgggatcccagcgcccgcactcccgcccccgccaaggagccaggaatggcacaactagagaggagcgccatctctggcttcagctctaagtccaggcgaaactcattcgcatatgatgttaagcgtgaagtatacaatgaggagacctttcaacaggaacacaaaaggaaggcctcctcttctgggaacatgaacatcaacatcaccaccttcagacaccacgtccagtgccgctgctcatggcacaggttcctacgatgcatgcttacaatctttcccttcctagaatggatgtgtatgtatcgattaaaggattggcttctgggagacttacttgctggtataagtgttggccttgtgcaagttccccaaggcctgacacttagtttgctggcaaggcaactgattcctcctctcaacatcgcttatgcagctttctgttcttcggtaatctatgtaatttttggatcgtgtcatcaaatgtccattggttccttcttcctggtgagtgctctgctgatcaacgttctgaaagtgagcccattcaacaacggtcaactggtcatgggatctttcgtcaagaatgagttttcggccccctcctaccttatgggctataataaatccttgagtgtggtggcaaccacaacttttctgactgggattattcagctaataatgggcgtattgggtttgggcttcattgccacttaccttccggagtctgcaatgagtgcttacctggctgctgtggcacttcatatcatgctgtcccagctgactttcatctttgggattatgattagtttccatgccggtcccatctccttcttctatgacataattaattactgtgtagctctcccaaaagcgaattccaccagcattctagtatttctaactgttgttgttgctctgcgaatcaacaaatgtatcagaatttctttcaatcagtatcccattgagtttcccatggaattatttctgattattggcttcactgtgattgcaaacaagataagcatggccacagaaaccagccagacgcttattgacatgattccttatagctttctgcttcctgtaacaccagatttcagccttcttcccaagataattttacaagccttctccttatctttggtgagctcctttctgctcatatttctgggcaagaagattgccagtcttcacaattacagtgtcaattccaaccaggatttaatagccatcggcctttgcaatgtcgtcagttcatttttcagatcttgtgtgtttactggtgctattgctaggactattatccaggataaatctggaggaagacaacagtttgcatctctggtaggcgcaggtgtgatgctgctcctgatggtgaagatgggacactttttctacacactgccaaatgctgtgctggctggtattattctgagcaacgtcattccctaccttgaaaccatttctaacctacccagcctgtggaggcaggaccaatatgactgtgctctttggatgatgacattctcatcttcaattttcctgggactggacattggactaattatctcagtagtttctgctttcttcatcaccactgttcgttcacacagagctaagattcttctcctgggtcaaatccctaacaccaacatttatagaagcatcaatgattatcgggagatcatcaccattcctggggtgaaaatcttccagtgctgcagctcaattacatttgtaaatgtttactacctaaagcataagctgttaaaagaggttgatatggtaaaggtgcctcttaaagaagaagaaattttcagcttgtttaattcaagtgacaccaatctacaaggaggaaagatttgcaggtgtttctgcaactgtgatgatctggagccgctgcccaggattctttacacagagcgatttgaaaataaactggatcccgaagcatcctccattaacctgattcactgctcacattttgagagcatgaacacaagccaaactgcatccgaagaccaagtgccatacacagtatcgtccgtgtctcagaaaaatcaagggcaacagtatgaggaggtggaggaagtttggcttcctaataactcatcaagaaacagctcaccaggactgcctgatgtggcggaaagccaggggaggagatcactcatcccttactcagatgcgtctctactgcccagtgtccacaccatcatcctggatttctccatggtacactacgtggattcacgggggttagtcgtattaagacagatatgcaatgcctttcaaaacgccaacattttgatactcattgcagggtgtcactcttccatagtcagggcatttgagaggaatgatttctttgacgctggcatcaccaagacccagctgttcctcagcgttcacgacgccgtgctgtttgccttgtcaaggaaggtcataggctcctctgagttaagcatcgatgaatccgagacagtgatacgggaaacctactcagaaacagacaagaatgacaattcaagatataaaatgagcagcagttttctaggaagccaaaaaaatgtaagtccaggcttcatcaagatccaacagcctgtagaagaggagtcggagttggatttggagctggaatcagaacaagaggctgggctgggtctggacctagacctggatcgggagctggagcctgaaatggagcccaaggctgagaccgagaccaagacccagaccgagatggagccccagcctgagactgagcctgagatggagcccaaccccaaatctaggccaagagctcacacttttcctcagcagcgttactggcctatgtatcatccgtctatggcttccacccagtctcagactcagactcggacatggtcagtggagaggagacgccatcctatggattcatactcaccagagggcaacagcaatgaagatgtctaggagatgaactagaaataaggggtcagataatgctggcaaatcctcctacccaaaaaggggtcaattgtccagagacctagactggatacgaactagcagtacttccttcctgactgtgactcctactacctgccagccttcttccttgctctgcgctgggatcatactcccaaatcacattactaaatgccaacaattatctctgaattccctatccaggctcccctcatttcaccttcagcatatattctagtcatgaatttccttcttcacacaccccacatctctgggctttgtgccagaccatctctaacttaatcctctcatccctgttcccctttctccaaagagatgaagctcaaataaaatgtataactctagtaaaaaa

SEQ ID NO: 28 - DNA, 540 bp, Homo sapiens
  misc_feature: Incyte ID No 7480432CB1
atgggaggcaagcccatgtgggagatgactggacccatcttcattcaacgttcagttattgagttctataacaatagaactcaactcagcacaatttacattgacatatcacgccttaggcgggaaggagagcagctcgaggggaaagctgccattgtgaagaagccatccagccttctgttccacaaaatccagcatagcatcatggtgcaggaccgtcagcccacaccagctaactgcatcctcagcatggttgtgagccagccaaatgccaatgaagaccccattatggggctccaccagatgttcctattaaaggacataatggatgcttgggttcgcctgatgacagacatgttcaggcctgccctgcacgacttcactgacctcctcccagccaggcactcacgctgtttctttctccctcctcttcccaatactattcacgcttctgcagacacaccagatactatacacaaatgcacggggctgtgtgggggcggacacggtgcactgttgccaccaaggtgtcctgcatga

SEQ ID NO: 29 - DNA, 5454 bp, Homo sapiens
  misc_feature: Incyte ID No 7494181CB1
cttgtcttgatcttatggccagtcattattttcataattttggctattactcggaccaaatttcctccaactgcaaaaccaacttgtccattttgcttctccctttataaagacatcattaacatgcccgctggacctgtgatttgggctttcttgaaacctatgttgttgggaagaattttgtatgcaccatataacccagtcacaaaggcaataatggaaaaggttggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaagagtccaaatttaaatgcaaatgcagtatgtgacaccatggccgtgacagcaatgatccaatcacatctccccgaagcctacctcaaggaggatattgggggagagcttgtttatgtacttcctccattcagcaccaaagtctcaggggcctacctgtcactcctacgggcactcgacaatggcatgggtgacctcaacatcgggtgctacggcatttcagataccaccgtggaggaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacacccgcaggaactggaaaggtctcattgctcaggttatcctccccatcgtctttgttaccactgccatgggccttggcacactgagaaattccagcaacagttatccagagattcagatctccccctctctttatggtacctccgaacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggtatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcattgggatacctgctggagagtgttttgggcttcttggagtgaatggagcaggaaagaccactatattcaagatgctgacaggagacatcattccttcaagtggaaacattctgatcagaaataagaccggatctttgggtcacgttgattctcgcagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaacacttccagcaaactcaatctcagcgtgtgaccaatggcttcattttgaagaaaagccacagaagatacacttccgcaagatatcttcattttaaagtaaagtaatatactgtatggaaagttacaactgtgttagactaacaagtaattataaaaggaaatttttccttctaaggtcagtgagtgttgttgctactgaaatgaattcctgtatactcaacactgtgagcatgctaatgtatatgctggtgattcttatgcaaaggtgaagccacctcaagatgaatatcttaatttattactttcaataaaaagaccagtttaaaaggccaaaaaaaaaaa

SEQ ID NO: 30 - DNA, 3670 bp, Homo sapiens
  misc_feature: Incyte ID No 3697053CB1
atgccttttaaagcatttgataccttcaaagaaaaaattctgaaacctgggaaggaaggagtgaagaacgccgtgggagattctttgggaattttacaaaaaaaatcgatgggacaactgagggaagaagataacattgagctgaatgaagaaggaaggccggtgcagacgtccaggccaagccccccactctgcgactgccactgctgcggcctccccaagcgttacatcattgctatcatgagtgggctgggattctgcatttcctttgggatccggtgcaatcttggagttgccattgtggaaatggtcaacaatagcaccgtatatgttgatggaaaaccggaaattcagacagcacagtttaactgggatccagaaacagtgggccttatccatggatcttttttctggggctatattatgacacaaattccaggtggtttcatttcaaacaagtttgctgctaacagggtctttggagctgccatcttcttaacatcgactctgaacatgtttattccctctgcagccagagtgcattacggatgcgtcatgtgtgtcagaattctgcaaggtttagtggaggaatcaatcaacaacagaacaacaacagcacatgccgctgccatcaacacagtggtaaatgtgtcgggggaaggggcccatgaaggttcctatgcaggggcagtggttgccatgcccctggctggggtgttggtgcagtacattggatggtcctctgtcttttatatttatggcatgtttgggattatttggtacatgttttggctgttgcaggcctatgagtgcccagcagctcatccaacaatatccaatgaggagaagacctatatagagacaagcataggagagggggccaacgtggttagtctaagtaaatttagtaccccatggaaaagatttttcacatctttgccggtttatgcaatcattgtggcaaatttttgcagaagctggaccttttatttgctcctcataagtcagcctgcttattttgaagaggtctttggatttgcaataagtaaggtgggtctcttgtcagcagtcccacacatggttatgacaatcgttgtacctattggaggacaattggctgattatttaagaagcagacaaattttaaccacaactgctgtcagaaaaatcatgaactgtggaggttttggcatggaggcaaccttactcctggtggttggcttttcgcataccaaaggggtggctatctcctttctggtacttgctgtaggatttagtggcttcgctatttcaggttttaatgtcaaccacctggacattgccccacgctatgccagcattctcatggggatctcaaacggagtgggaaccctctctggaatggtctgtcccctcattgtcggtgcaatgaccaggcacaagacccgtgaagaatggcagaatgtgttcctcatagctgccctggtgcattacagtggtgtgatcttctatggggtctttgcttctggggagaaacaggagtgggctgacccagagaatctctctgaggagaaatgtggaatcattgaccaggacgaattagctgaggagatagaactcaaccatgagagttttgcgagtcccaaaaagaagatgtcttatggagccacctcccagaattgtgaagtccagaagaaggaatggaaaggacagagaggagcgacccttgatgaggaagagctgacatcctaccagaatgaagagagaaacttctcaactatatcctaatgtctgagaggcacttctgtcttctccttactttagaaccagaaagtatccatacctattgcctttcttgtagcccagcttgccagaggtccaaatattgggaggggagaagatctaaccagcaacagggaaaagagaaatattatctttcaatgacatgtataggtaaggagctgcgctcagttgataacatagttgataatacatattttttgaattgacagttgacccttctctcaaagagctaaacttattcagaaaggaatgactagaagaaaaaggagacaataccatgttgttcaaagaaacattgaaggaaattgggatgtttggccagaaggaatgtaaacagtagtagtagctgccaccacatctctagggtagccatgcagaggagggcttcatattcccaataaaccccacgttgtggcaggtgctttataaacactcttatttaatctccacacctttatgacacacatttcttatccccattttacaaccaaggcatctaaagcaacaagaaatgaacttgcccaaggtcatctgccagggtcagtgctgagactgttgaagctctcaataggtggcagttttagggaagatttccattcagtgtagggaagacatttgtaataatgaaaactgaaaatggagtaattgtgagtaactcaccactttagcaggtgttggggaagggaaacatttgggttgatgaggcagaggggattcaaatgtgtgagaggctagattcaaagaccctcagtgttctatgttatctgaagagtcaaatggttttgtgactccatagtttttaaagtaataagggtcaaagactacatcagagattcaaataggtttttaaagaaaagctaagcaagagagccaaatttttagaaatctgatggtcaaaatagctgaaagcagtaaacaagagattggctattaaatttcaactttccataatattaagaatgtagctaaatgatgtcccaaactacttacaaacttttaagacatttaataatttaagaagtaggttcatgtgttttcttaggtaaagttcttctgaaagaattttctatttttaaaaaatgtatctctttagccttttctgctggagattatattaggaagtttcatcagattgtataaaattatgattttgtatcaaaagtattcatgatgactctatttggaatgatattcagggaaatcacaataatatagcagtagttatacagagaaatactacaatgaaaacatttggggcaattagacctacagttactgttgaaaaattcacctttgattgcataaggcaattacatggatacttttagatatatttaaaattttaacattggcatctaaagtgttatttgaaaataaaattattttcctgttcattgattttaaacattttattcctactttcagaagaaaaatataatacggaaaaaattatagatttacttgtagcttattattgtaaagtgtttttttttttttctaatttctcccacatgtatttctggtccccagtgatactagctgagttgtagtgtattttataaatggaataatcttggggaaaaattgcgattcttcattaaataatattctttatgtcactagcatacaatttatgttagtagacatctttaaatctctttaatgagtgaatccatgcaagccccataaaacagttcctagcatgcagaaaatgcccacgtaaatagctgtcatcatcattatcttttaacattttgggggactttccagttgaaaagaaaacatgctatgtcatttttatccattatccctggaacttattgtgaaagttgtgctgttttctaagtaaaataaaaaataaaaaattagccaatttaaaaaaaaaaaa

SEQ ID NO: 31 - DNA, 1009 bp, Homo sapiens
  misc_feature: Incyte ID No 7473203CB1
aacctgcacatgtacccactgaacctaaaataaaagttaacacaaaaaggaaaaagtttattaagtaaaaaaattagaagaagctaaggttaatttattattgaagaaagagatttcacttcacggcagctgtggcctgactgtgaggcggctgaccagcctcagctgagagtggagtggtggccgtggccctccttaggagatcaccatatcacctcttactgtggccttaccatggcgacctatgggcagacctgcatgtggccagtgtggatttcttcatcatatgttaaccttggcaaagctgccagagatatttttaacaaaggatttggtttggggttggtaaaactggatgtgagaacaaagtcacgcagtgctgtgggattttcaacatctggttcatttaatgcagacactggaaaagcttttgaagtcttggagaccaaatataaacggtcaatcacaggaaacaaaagtggtaaaatcaagtcctcttgcaagagggactgcataaaccttgcttgtgatgttaattttgattttgctggacctgcaatctatgcttcagctgtctttggttacgagggctggcttgctgggtaccagatgaccactgacagtgccaagtcaaagctgacaaggaataactgtagtgggtaccggatgggggacttcgagcttcacactaataacaataatggggcagaatttggaggctcagtttatcagagggtatgtgacaatcttgatacttcagtaaaccttgctcggacatcaagtgccaactgcactttttgtcttgccactaaatatcagttgcatttcactgcttctatgtttgcaaaagtcaacaactctagtttaattggagtggaaggaaagagacttcatttacactcagactctgagcctgctgtgaagcttgcactttctgctctgctagataaaaagtgcattaatggaggaggccaaagacttgggtttgtcctggagttggagacttaat

SEQ ID NO: 32 - DNA, 2398 bp, Homo sapiens
  misc_feature: Incyte ID No 4697002CB1
gccgcccagtccgagggcgcagagcgccaggagcacgcggagggctggggcgcgggctccgggaacgagaaagtgcagctctctcgggtcactgggccggcggcggggggactatggctctgaaggacacgggcagcggcggcagcaccatcctgcccattagcgagatggtttcctcgtccagctcgcccggcgcgtcggccgccgccgccccggggccctgcgcaccctcgcccttccctgaagtagtggagctgaacgtaggcggccaggtttatgtgaccaagcactcgacgctgctcagcgtcccggacagtactttggccagcatgttctcgccctctagtccccgtggcggcgcccggcgccggggcgagctgcccagggacagccgggcgcgcttcttcatcgaccgggacggcttccttttcaggtacgtgctggattatctgcgggacaagcaactcgcgctgccggagcacttccccgagaaggagcggctgctgcgcgaggccgagtatttccagctcaccgacttggtcaagctgctgtcgcccaaggtcaccaagcagaactctctcaacgacgagggctgccagagcgacctggaggacaacgtctcgcagggtagcagcgacgcgctgctgctgcgcggggcggcggccgccgtgccctcgggcccgggagcgcacggtggtggcggcggcggcggcgcgcaggacaagcgctcgggcttcctcacgctgggctaccggggctcctacaccaccgtgcgcgacaaccaggccgacgccaaattccggcgtgtggcgcgcatcatggtgtgcgggcgcatcgcgctggccaaggaggtcttcggggacacgctcaacgagagccgcgaccccgaccggcagccggagaagtacacgtcccgcttctacctcaagttcacctacttggagcaggcctttgatcgcctgtccgaggccggcttccacatggtggcgtgtaactcctcgggcaccgccgccttcgtcaaccagtaccgcgacgacaagatctggagcagctacaccgagtacattttcttccgaccacctcagaaaatagtatcacctaaacaagaacatgaagataggaaacatgacaaagtcactgataaaggaagtgaaagtgggacttcctgtaatgagctctccacttccagttgtgacagccattcagaggcaagcactccccaggacaacccatccagtgcccagcaggcaacagctcaccaacctaacactttaacattggatcgcccctctaaaaaagcacctgtacaatggatacccccaccagacaaacgcagaaacagtgaactctttcagaccctcatcagcaagtcccgggaaacaaatctgtccaaaaagaaagtctgtgagaagctaagtgtggaagaagaaatgaaaaagtgtattcaggattttaaaaaaatccacattccagattattttccagagcgcaaacgccaatggcaatctgaactgttgcagaagtatgggttatagtaattgtcacattcctgcagtattttgatgacattcaatgtttactacagtgtcaccacctgactgatgtcctaacaatggtcagtgtgattcttgctgctcttccttgttgtgaacagtggatgtgggacagtattttcttttatgttttagttgttgttctttttagaaacatgattaaaaaggaaaaaatattaaatcaataagtgttaaatcaaaatggaatatctgattcaaaccattttacaagaatgaaagtaaaatgtgcatgatcaagcttagtatcttggtttttgaactctggtcaactggatatgtttgtcattttgtaacttaccaaaaacaaaccatcatatcataccaactaaaatgatatatggatgaagcaacatcaagtaaaattttagacgatggctataggacccaaatctaaagctgtctaaatgttaattcaatgaaacaagtattatttttgcatgaatacaatgttacaaataaatcacaagaaatagggaagatctgtttgttgcttggaaagaaaaaaattacaaaaaaaagcaaaaaaaaatgttttagacaaagcctataaatgtaagctgtctaggagattgaattttctttgttctggatctgtgacttttttgtgtatgtgtatggtgtttatgtatattaagatggtgtaaatatgccttatactgttatttatggcatcaacattcatttactaatgctagtcatgattattactgtgaaatgagtcttacatcgggctgtgaaaattggtataatgatgctttgaaagatcctattatcatgttaatcaaaataatagagggaaatggtaaagagctttatgtatttat

SEQ ID NO: 33 - DNA, 4160 bp, Homo sapiens
  misc_feature: Incyte ID No 5632139CB1
gcaaacgcgcggcctactacagcgccgcggggcccaggccgggagccgaccggcacagcaggtaccagctggaggatgagtctgcgcatttggatgaaatgccactaatgatgtctgaagaaggctttgagaatgaggaaagtgattaccacaccttaccacgagccaggataatgcaaaggaaaagaggactggagtggtttgtctgtgatggctggaagttcctctgtaccagttgctgtggttggctgataaatatttgtcgaagaaagaaagagctgaaagctcgcacagtatggcttggatgtcctgaaaagtgtgaagaaaaacatcccaggaattctataaaaaatcaaaaatacaatgtgtttacctttatacctggggttttgtatgaacaattcaagtttttcttgaatctctattttctagtaatatcctgctcacagtttgtaccagcattgaaaataggctatctctacacctactgggctcctctgggatttgtcttggctgttactatgacacgggaagcaattgatgaatttcggcgttttcagcgtgacaaggaagtgaattcacaactatatagcaagcttacagtaagaggtaaagtgcaagttaagagttcagacatacaagttggagacctcatcatagtggaaaagaatcaaagaattccatcggacatggtgtttcttaggacttcagaaaaagcaggttcgtgttttattcgaactgatcaactagatggtgaaactgactggaagctgaaggtggcagtgagctgcacgcaacagctgccgactctgggggaccttgtttctatcagtgctaatgtttatgctcagaaaccacaaatggacattcacagtttcgaaggcacatttaccagggaagacagtgacccgcccattcatgaaagtctcagcatagaaaatacattgtgggcaagcaccattgttgcatcaggtactgtaataggtgttgtcatttataccggaaaagagactcgaagtgtaatgaacacatccaatccaaaaaataaggttggtttgttggaccttgaactcaatcggctgacgaaagcgctatttttggctttagttgctctttccattgttatggtaaccttacaaggatttgtgggtccatggtaccgcaatctttttcggttccttctcctcttttcttacatcattcccataagtttgcgtgtgaacttggacatgggcaaagcggtgtatggatggatgatgatgaaagatgagaacatccctggcacggtcgttcggaccagcactatcccagaggaacttgggcgcctggtgtatttattgacagacaaaacaggaaccctcacccagaatgaaatgatatttaagcggctgcacctgggcaccgtgtcctatggcgccgacacgatggatgagatccagagccatgtcagggactcctactcacagatgcagtctcaagcgggtggaaacaatactggttcaactccactaagaaaagcccaatcttcagctcccaaagttaggaaaagtgtcagtagtcgaatccatgaagccgtgaaagccatcgtgctgtgtcacaacgtgacccccgtgtatgagtctcgggccggcgttactgaggagactgagttcgcagaggctgaccaagacttcagtgatgagaatcgcacctaccaggcttccagcccggatgaggtcgctctggtgcagtggacagagagtgtgggcctcacgctggtcagcagggacctcacctccatgcagctgaagacccccagtggccaggtcctcagcttctgcattctgcagctgtttcccttcacctccgagagcaagcggatgggcgtcatcgtcagggatgaatccacggcagaaatcacattctacatgaagggcgctgacgtggccatgtctcctatcgtgcagtataatgactggctggaagaggagtgcggaaacatggctcgcgaaggactgcggaccctcgtggttgcaaagaaggcgttgacagaggagcagtaccaggactttgagagccgatacactcaagccaagctgagcatgcacgacaggtccctcaaggtggccgcggtagtcgagagcctggagagggagatggaactgctgtgcctcaccggcgtggaggaccagctgcaggcagacgtgcggcccacgctggagatgctgcgcaacgccgggatcaagatatggatgctaacaggcgataaactcgagacagctacctgcattgccaaaagttcacatctcgtgtctagaacacaagatattcatattttcagacaggtaaccagtcggggagaggcacatttggagctgaatgcatttcgaaggaagcatgattgtgcactagtcatatctggggactctctggaggtttgtctaaagtactacgagcatgaatttgtggagctggcctgccagtgccctgccgtggtttgctgccgctgctcacccacccagaaggcccgcattgtgacactgctgcagcagcacacagggagacgcacctgcgccatcggtgatggaggaaatgatgtcagcatgattcaggcagcagactgtgggattgggattgagggaaaggagggtaaacaggcctcgctggcggccgacttctccatcacgcagttccggcacataggcaggctgctcatggtgcacgggcggaacagctacaagaggtcggcggcactcggccagttcgtcatgcacaggggccttatcatctccaccatgcaggctgtgttttcctcagtcttctacttcgcatccgtccctttgtatcagggcttcctcatggtggggtatgccaccatatacaccatgttcccagtgttctccttagtgctggaccaggacgtgaagccagagatggcgatgctctacccggagctgtacaaggacctcaccaagggaagatccttgtccttcaaaaccttcctcatctgggttttaataagtatttaccaaggcggcatcctcatgtatggggccctggtgctcttcgagtctgagttcgtccacgtggtggccatctccttcaccgcactgatcctgaccgagctgctgatggtggcgctgaccgtccgcacgtggcactggctgatggtggtggccgagttcctcagcttaggctgctacgtgtcctcactcgcttttctcaatgaatattttggtataggcagagtgtcttttggagctttcttagatgttgcctttatcaccaccgtgaccttcctgtggaaagtgtcggcgatcaccgtggtcagctgcctcccgctgtatgtcctcaagtacctgaggcgcaagctctctcctcccagctactgcaagctggcctcctaaggggctgtgcacccccagcgggctggccccagcaccttctgcccttcccagcaccttgtgcccttgccagtgaacgcagggtttgccattgctaccaagcaagcaccacaagaaagggagggtacgccaggcgagcccagggcacagatgctgagacagcctctccttctcagtgcagggacgtcacccctgccaggcaagcccagggcacagatgccaggatggcttctccctctcagtgcgaggcttcacccctgccaggcaagcccagggcatagatgctgagacagcctctccctctcagtgcagggacgtcacccctgccaggcaagcccagggcacagaggccgggacggcctctccctctcagtgtgaggcttcacccatgctaggcaagcccagggcacagatgccgggatggcccctccctctcagtgcgggaacgtcacccctgccaggcaagcccagggcacagatgctgcgatggcctcttcctcttaagtgtggggcctcacccctgcttttctttctttttttgtattgtcaaaattgtatttccatattgaagcagcttgagtttctactgaaaatgagcccgaattatttcactattactgtaaagggttcatcttactctggcattctgagaatcagactgaaagtttaatttctgcagttccctcacattcagattctttctttgatgttataacacaaagtcattcctactcaaatgtaataaaattgaggctccacggagaaaaaaaaacaaaa

SEQ ID NO: 34 - DNA, 2835 bp, Homo sapiens
  misc_feature: Incyte ID No 7506184CB1
gcgatccaaacgccctggctctcaggcctggactctagggcttagccagatgcctaaaccgcccaagccgagaaacaacttagaagacagacataaccctgggattcagggaaggcgcgagcaccgcccaggacctggtagggtgcgagccgcgagcagtccgggagggagcgcgcctagggcggagcgtaggctgtggggggagggctgggagtccggggccgccccacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctacaaaaaaaaaaaaaaaaaaaaaaaaaaaaaaaaaaaaataaaaaaaaaaggggggcccccctaaggggtccccaactntgcctggggggcattgnggttanaaccccttttaannnnnnnnnnnnatttatttccgnnngggttnnntaanaagggggtgggggnnnaccnnnnnngttnnnncaatnttaccnctttcacccccccccctgnnccttttggaccnnnnnnnnnnnnnnnnnnnnnnnnnnnnnnnnnnnnnnnnnnnnnnnnnnnnnnnttnnnnnnnnnnnatttctcgcctttggtcacaggttgctgacgaatagagggatgctttctctttatcccgcacc